(19) United States
(12) Patent Application Publication
(10) Pub. No.: US 2024/0156729 A1
(43) Pub. Date: May 16, 2024

(54) FORMULATIONS FOR AEROSOL FORMATION AND AEROSOLS FOR THE DELIVERY OF NUCLEIC ACID

(71) Applicant: ethris GmbH, Planegg (DE)

(72) Inventors: Christian Dohmen, Neuried (DE); Philipp Beck, München (DE); Christian Plank, Weßling (DE)

(21) Appl. No.: 18/277,965

(22) PCT Filed: Feb. 25, 2022
(86) PCT No.: PCT/EP2022/054796

§ 371 Date: Aug. 18, 2023

(30) Foreign Application Priority Data
Feb. 26, 2021 (EP) 21159455.1

(51) Int. Cl. A61K 9/00; A61K 9/51; A61K 31/7105; A61K 47/10; A61K 47/26; C12N 15/88

(52) U.S. Cl. CPC A61K 9/0078; A61K 9/5123; A61K 9/5146; A61K 31/7105; A61K 47/10; A61K 47/26; C12N 15/88

(57) ABSTRACT

The invention relates to an aqueous suspension formulation for aerosol formation, said suspension formulation comprising lipid or lipidoid nanoparticles which are suspended in an aqueous vehicle solution,

wherein the lipid or lipidoid nanoparticles comprise the following components (a) and (b):
(a) a nucleic acid and
(b) an ionizable lipid or an ionizable lipidoid;
and wherein the aqueous vehicle solution comprises a triblock copolymer which contains one poly(propylene oxide) block and two poly(ethylene oxide) blocks. Moreover, the invention relates to an aerosol obtained from the formulation for aerosol formation.

The present invention relates to aqueous suspension formulations for aerosol formation and to aerosols which can be advantageously used for the administration of a nucleic acid to a subject.

Lipid formulations such as lipid vesicles including liposomes, and lipid or lipidoid nanoparticles (LNPs) are frequently used for the delivery of active pharmaceutical ingredients in patients. In particular, lipid or lipidoid formulations of nucleic acids are extremely useful and efficient for introducing nucleic acids into cells. This advantageous property of lipid or lipidoid formulations of nucleic acids has been used for decades in biological and medical research and in therapeutic approaches to i) overexpress genes or to complement genetic defects in target cells, or ii) to downregulate or upregulate endogenous gene expression in cells, or iii) to repair genetic defects (mutations).

For the overexpression of genes and complementation of genetic defects, nucleic acids that contain sequences coding for proteins are introduced into cells. These are either DNA constructs comprising a coding region under the control of a suitable promoter, which are transcribed into mRNA in the nucleus of a cell. The mRNA translocates to the cytoplasm where it is translated into a protein. Alternatively, in vitro transcribed mRNA can be introduced into the cytoplasm using lipid formulations to achieve the same effect. In gene therapy and mRNA transcript therapy, the concept of introducing exogenous genetic information into patient cells is exploited to induce patient cells to produce a protein that has a therapeutic effect.

For the downregulation of endogenous gene expression, fully synthetic nucleic acids such as, among others, synthetic (antisense) oligonucleotides or siRNAs or ribozymes can be used, or (plasmid) DNA constructs which are transcribed into RNAs in the cell that are suitable for downregulating endogenous gene expression. For knockdown of endogenous gene expression nucleic acids can be used that code for nucleases such as zinc finger nucleases, TALE nucleases, or the CRISPR-Cas system. Similarly, upregulation of endogenous gene expression can be achieved with certain oligonucleotides by various mechanisms (Khorkova O, Hsiao J, Wahlestedt C. Oligonucleotides for upregulating gene expression. Pharm Pat Anal. 2013;2(2):215-29; Sargent R G, Kim S, Gruenert D C. Oligo/polynucleotide-based gene modification: strategies and therapeutic potential. Oligonucleotides. 2011;21(2):55-75), an approach also known as therapeutic gene modulation. A special case of therapeutic gene modulation is immune stimulation with CpG motif-containing oligonucleotides (Krieg A M. CpG motifs in bacterial DNA and their immune effects. Annu Rev lmmunol. 2002;20:709-60).

For the repair of genetic defects at the mRNA level, nucleic acid constructs that influence splicing reactions can be used, such as but not limited to oligonucleotides for exon skipping. For gene repair at the genomic level, nucleic acids can be used that code for nucleases that can change nucleic acid sequences in chromosomes, such as zinc finger nucleases, TALE nucleases, or the CRISPR-Cas system.

In any of the three concepts of nucleic acid therapy (overexpression-gene complementation/downregulation or upregulation of endogenous genes/gene repair), lipid formulations of nucleic acids need to be introduced into the patient's body in a manner that is tolerable for the patient and that is suitable for the nucleic acid to exert its desired effect in target cells or in target organs or throughout the patient's body. Routes of administration that are frequently used include local injection, such as intradermal, subcutaneous, intra-ocular, intramuscular, intra-myocardial, intra-tumoral or direct administration in other target tissue or organ and furthermore systemic administration (usually intravenous).

Less frequently used is administration of an aerosol of a lipid or lipidoid formulation of an active pharmaceutical ingredient, in particular of a nucleic acid, by inhalation. While this route of administration appears most convenient and useful to deliver an active pharmaceutical ingredient, in particular a nucleic acid, to target cells in the respiratory tract, this route of administration is associated with numerous challenges, particularly in case of lipid or lipidoid formulations of nucleic acids, as outlined below.

The challenges result on the one hand from the requirements of the medical application. It must be ensured that, when administered by inhalation, a therapeutically effective dose is deposited within a reasonable period of time in those areas of the respiratory tract of a patient where it can develop the desired therapeutic efficacy. Depending on the medical application, this can be, for example, either the upper respiratory tract or the alveolar region. Moreover, as far as the time required for the necessary dose deposition is concerned, patient compliance must be taken into account. Inhalation times of an hour or more certainly constitute a high burden to patients while 30 minutes or less are obviously more convenient.

Further challenges result from regulatory requirements as outlined in the FDA Guidance for Industry entitled “Liposome Drug Products Chemistry, Manufacturing, and Controls; Human Pharmacokinetics and Bioavailability; and Labeling Documentation” (Liposome Drug Products Chemistry, Manufacturing, and Controls; Human Pharmacokinetics and Bioavailability; and Labeling Documentation—Guidance for Industry, (2018)) and in EMA Guideline EMEA/CHMP/QWP/49313/2005 Corr, “Guideline on the Pharmaceutical Quality of Inhalation and Nasal Products”, 2006. Both Guidelines emphasize the importance of the integrity of the drug product. Physicochemical properties such as vesicle/particle size and size distribution, and morphology are listed as Critical Quality Attributes. “Development studies should include physical characterisation of drug substance and excipients, relevant to their effect on the functionality of the product” (Guideline on the Pharmaceutical Quality of Inhalation and Nasal Products, (2006)). EMEA/CHMP/QWP/49313/2005 Corr states that “physical characteristics such as solubility, size, shape, density, rugosity, charge, and crystallinity of the drug substance and/or excipients may influence the homogeneity and reproducibility of the finished product”. The Guideline requires that delivered dose uniformity and fine particle mass be warranted over patient flow rate range. Susceptibility to fusion (i.e., irreversible coalition of smaller liposomes to form larger liposomes), aggregation (i.e., reversible conglomeration or pooling of two or more liposomes without fusion), and leakage of the contained drug substance during storage can affect the stability of the drug product (Liposome Drug Products Chemistry, Manufacturing, and Controls; Human Pharmacokinetics and Bioavailability; and Labeling Documentation—Guidance for Industry, (2018)). In other words, the drug product present in a liquid to be nebulized should not change during nebulization, i.e. its composition, particle size and encapsulation efficiency of the drug substance (in the case of a particulate drug product) and its efficiency should remain unchanged.

For administration by inhalation, the lipid formulation of an active pharmaceutical ingredient needs to be nebulized. Various types of nebulizers known to the one skilled in the art are available for medical use. No matter what the design of the nebulizer, the nebulization of a liquid requires the introduction of considerable energy into the liquid. It has been observed that lipid or lipidoid formulations of pharmaceutically active ingredients do change in size, morphology and encapsulation of drug substance when nebulized (Elhissi A M, Faizi M, Naji W F, Gill H S, Taylor K M. Physical stability and aerosol properties of liposomes delivered using an air-jet nebulizer and a novel micropump device with large mesh apertures. Int J Pharm. 2007;334(1-2):62-70; Li Z, Zhang Y, Wurtz W, Lee J K, Malinin V S, Durwas-Krishnan S, et al. Characterization of nebulized liposomal amikacin (Arikace) as a function of droplet size. J Aerosol Med Pulm Drug Deliv. 2008;21(3):245-54). Furthermore, it has been observed that a lipid formulation of mRNA can clog a nebulizer during nebulization and that the formulation loses efficacy upon nebulization.

Accordingly, the task underlying the present invention was providing a composition comprising a nucleic acid, which composition is suitable for delivering a nucleic acid to a cell in the respiratory tract in a subject upon nebulization and inhalation of said composition in a manner that the nucleic acid efficiently exerts its intended function following the inhalation, such as leading to the production of a protein in a lung cell, leading to the downregulation or upregulation of expression of an endogenous gene or leading to gene repair. Moreover, it should be possible to nebulize an effective dose of the composition within a reasonable period of time, and the components of the composition should remain intact during nebulization.

In the context of the present invention, it was found that a suspension formulation of lipid or lipidoid nanoarticles (LNPs) which comprise a nucleic acid and an ionizable lipid or an ionizable lipidoid, and which are suspended in an aqueous vehicle solution comprising a poly(ethylene oxide)-poly(propylene oxide) block copolymer can be efficiently nebulized while a negative impact of the nebulization procedure on the integrity of the nanoparticles and the nucleic acid contained therein can be prevented. In particular, the use of this formulation for the preparation of an aerosol allows a significantly improved resistance of the particles against aggregation during the nebulization process and a beneficial retention of the transfection efficiency of the nucleic acid after the nebulization process to be achieved.

A summary of the aspects of the invention is provided in the following items.

1. An aqueous suspension formulation for aerosol formation, said suspension formulation comprising lipid or lipidoid nanoparticles which are suspended in an aqueous vehicle solution, wherein the lipid or lipidoid nanoparticles comprise the following components (a) and (b):
(a) a nucleic acid and
(b) an ionizable lipid or an ionizable lipidoid;
   and wherein the aqueous vehicle solution comprises a triblock copolymer which contains one poly(propylene oxide) block and two poly(ethylene oxide) blocks.

2. The suspension formulation in accordance with item 1, wherein the nucleic acid is selected from RNA and plasmid DNA.

3. The suspension formulation in accordance with item 1 or 2, wherein the nucleic acid is selected from mRNA, siRNA, miRNA, antisense RNA, tRNA, and noncoding RNA and is more preferably mRNA.

4. The suspension formulation in accordance with any of items 1 to 3, wherein the concentration of the nucleic acid in the suspension formulation ranges from 0.01 to 10 mg/mL, more preferably from 0.02 to 10 mg/mL, and most preferably from 0.05 to 5 mg/mL, based on the total volume of the suspension formulation.

5. The suspension formulation in accordance with any of items 1 to 4, wherein the weight-to-volume ratio of the nanoparticles in the aqueous suspension in gram per liter is in the range of 0.5 g/L to 100 g/L, preferably 10 g/L to 100 g/L, more preferably 10 g/L to 50 g/L and most preferably 10 g/L to 75 g/L.

6. The suspension formulation in accordance with any of items 1 to 5, wherein the nanoparticles have a Z-average diameter, as determined by dynamic light scattering, in the range of 10 to 500 nm, more preferably in the range of 10 to 250 nm, still more preferably 20 to 200 nm.

7. The suspension formulation in accordance with any of items 1 to 6, wherein the nanoparticles have a polydispersity index, as determined by dynamic light scattering, in the range of 0.05 to 0.4, more preferably in the range of 0.05 to 0.2.

8. The suspension formulation in accordance with any of items 1 to 7, wherein the nanoparticles further comprise one or more of the following components (c1) to (c6):
(c1) a non-ionizable lipid having a sterol structure;
(c2) a phosphoglyceride lipid;
(c3) a PEG-conjugated lipid;
(c4) a polysarcosine-conjugated lipid
(c5) a PASylated lipid; and
(c6) a cationic polymer.

9. The suspension formulation in accordance with any of items 1 to 8, wherein the nanoparticles comprise:
30 to 65 mol % of the ionizable lipid or ionizable lipidoid (b), and one or more of the following components:
10 to 50 mol % of the lipid having a sterol structure (c1),
4 to 50 mol % of the phosphoglyceride lipid (c2),
0.5 to 10 mol % of one of the PEG-conjugated lipid (c3), the polysarcosine-conjugated lipid (c4) and the PASylated lipid (c5), or of any combination thereof,
0.5 to 10 mol % of the cationic polymer (c6),
   such that the sum of (b) and (c1) to (c6) amounts to 100 mol %.

10. The suspension formulation in accordance with any of items 1 to 9, wherein the nanoparticles further comprise the following components (c1) to (c3):
(c1) a non-ionizable lipid having a sterol structure;
(c2) a phosphoglyceride lipid; and
(c3) a PEG-conjugated lipid.

11. The suspension formulation in accordance with item 10, wherein the nanoparticles comprise:
30 to 65 mol % of the ionizable lipid or ionizable lipidoid (b),
10 to 50 mol % of the lipid having a sterol structure (c1),
4 to 50 mol % of the phosphoglyceride lipid (c2), and
0.5 to 10 mol % of the PEG-conjugated lipid (c3),
   such that the sum of (b) and (c1) to (c3) amounts to 100 mol %.

12. The suspension formulation in accordance with any of items 1 to 11, wherein the nanoparticles further comprise a polyanionic component which is different from the nucleic acid.

13. The suspension formulation in accordance with any of items 1 to 12, wherein the composition of the nanoparticles is such that the weight ratio in the nanoparticles of the sum of the weights of components other than the nucleic acid to the weight of the nucleic acid is in the range of 30:1 to 1:1, more preferably 20:1 to 2:1 and most preferably 15:1 to 3:1.

14. The suspension formulation in accordance with any of items 1 to 13, wherein the nanoparticles comprise an ionizable lipidoid (b) of the following formula (b-1),

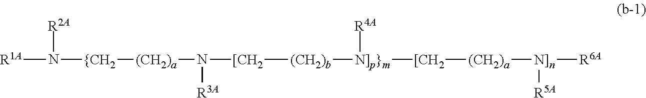

wherein:
a is 1 and b is an integer of 2 to 4; or a is an integer of 2 to 4 and b is 1,
p is 1 or 2,
m is 1 or 2; n is 0 or 1 and m+n is≥2; and
R_{1A} to R_{6A} are independently of each other selected from:
hydrogen; —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A}; —CH₂—R_{7A}; —C(NH)—NH₂; a poly(ethylene glycol) chain; and a receptor ligand; wherein R_{7A} is selected from C3-C18 alkyl and C3-C18 alkenyl having one C—C double bond;
provided that at least two residues among R_{1A} to R_{6A} are selected from —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A} and —CH₂—R_{7A}, wherein R_{7A} is selected from C3-C18 alkyl or C3-C18 alkenyl having one C—C double bond;
   or a protonated form thereof, wherein one or more of the nitrogen atoms contained in the compound of formula (b-1) are protonated to provide a compound carrying a positive charge.

15. The suspension formulation in accordance with any of items 1 to 14, wherein the nanoparticles comprise an ionizable lipidoid (b-1) of the following formula (b-1b),

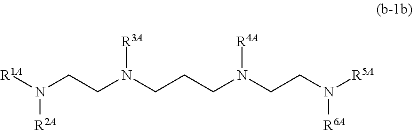

wherein R_{1A} to R_{6A} are defined as in item 14,
   or a protonated form thereof, wherein one or more of the nitrogen atoms contained in the compound of formula (b-1b) are protonated to provide a compound carrying a positive charge.

16. The suspension formulation in accordance with item 14 or 15, wherein R_{1A} to R_{6A} are independently selected from hydrogen and —CH₂—CH(OH)—R_{7A}, wherein R_{7A} is selected from C8-C18 alkyl and C8-C18 alkenyl having one C—C double bond, provided that at least two residues, preferably at least three residues, and more preferably at least four residues, among R_{1A} to R_{6A} are —CH₂—CH(OH)—R_{7A} wherein R_{7A} is selected from C8-C18 alkyl and C8-C18 alkenyl having one C—C double bond.

17. The suspension formulation in accordance with any of items 8 to 16, wherein the nanoparticles comprise a non-ionizable lipid having a sterol structure (c1) of formula (c1-1):

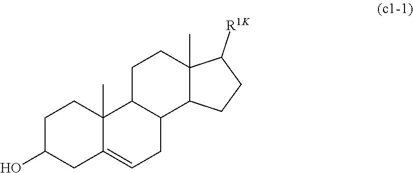

wherein R_{1K} is a C3-C12 alkyl group.

18. The suspension formulation in accordance with any of items 8 to 17, wherein the non-ionizable lipid having a sterol structure (c1-1) comprises cholesterol.

19. The suspension formulation in accordance with any of items 8 to 18, wherein the nanoparticles comprise a phosphoglyceride lipid (c2) of formula (c2-1)

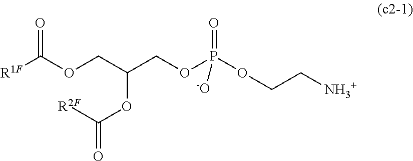

wherein
R_{1F} and R_{2F} are independently a C8-C18 alkyl group or a C8-C18 alkenyl group,
preferably a C12-C18 alkyl group or a C12-C18 alkenyl group,
   or a pharmaceutically acceptable salt thereof;
   or a phosphoglyceride lipid (c2) of formula (c2-2)

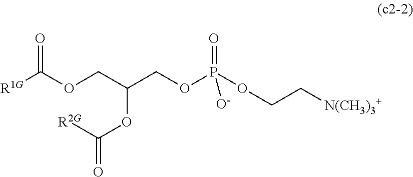

wherein
R_{1G} and R_{2G} are independently a C8-C18 alkyl group or a C8-C18 alkenyl group,
preferably a C12-C18 alkyl group or a C12-C18 alkenyl group,
   or a pharmaceutically acceptable salt thereof.

20. The suspension formulation in accordance with any of items 8 to 19, wherein the phosphoglyceride lipid (c2) comprises 1,2-dipalmitoyl-sn-glycero-3-phosphocholine (DPPC) or a pharmaceutically acceptable salt thereof.

21. The suspension formulation in accordance with any of items 8 to 20, wherein the nanoparticles comprise a PEG-conjugated lipid (c3) of formula (c3-1)

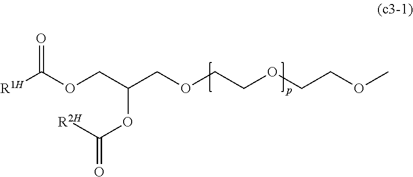

wherein
R_{1H} and R_{2H} are independently a C8-C18 alkyl group or a C8-C18 alkenyl group, preferably a C12-C18 alkyl group or a C12-C18 alkenyl group, and p is an integer of 5 to 200, preferably 10 to 100, and more preferably 20 to 60;
   or a PEG-conjugated lipid (c3) of formula (c3-2)

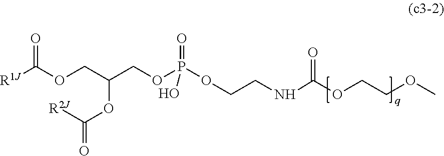

wherein
R_{1J} and R_{2J} are independently a C8-C18 alkyl group or a C8-C18 alkenyl group, preferably a C12-C18 alkyl group or a C12-C18 alkenyl group, and q is an integer of 5 to 200, preferably 10 to 100, and more preferably 20 to 60,
or a pharmaceutically acceptable salt thereof.

22. The suspension formulation in accordance with item 21, wherein the PEG conjugated lipid (c3) comprises 1,2-dimyristoyl-sn-glycerolmethoxy(polyethylene glycol)-2000 (DMG-PEG2k).

23. The suspension formulation in accordance with any of items 1 to 22, wherein the N/P ratio in the nanoparticles is in the range of 0.5 to 20, more preferably in the range of 0.5 to 10.

24. The suspension formulation in accordance with any of items 1 to 23, wherein the triblock copolymer is an A-B-A triblock copolymer which contains one poly(propylene oxide) block B of formula (p-1):

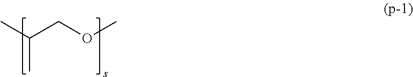

wherein s is an integer of 15 to 67, preferably 20 to 40 and
two poly(ethylene oxides) blocks A of formula (p-2):

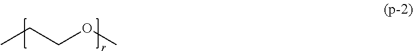

wherein r is, independently for each block, an integer of 2 to 130, preferably 50 to 100, and more preferably 60 to 90.

25. The suspension formulation in accordance with item 24, wherein the triblock copolymer has the following structure:

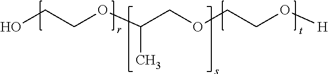

wherein r and t are independently of each other integers of 2 to 130, preferably 50 to 100, and more preferably 60 to 90, and s is an integer of 15 to 67, preferably 20 to 40.

26. The suspension formulation in accordance with item 24 or 25, wherein the triblock copolymer is Poloxamer P188.

27. The suspension formulation in accordance with any of items 1 to 26, which comprises the triblock copolymer at a concentration of 0.05 to 5% (w/v, at a temperature of 25° C.), preferably 0.1 to 2%, based on the total volume of the suspension formulation.

28. The suspension formulation in accordance with any of items 1 to 27, wherein the vehicle solution further comprises at least one of sucrose or NaCl, more preferably sucrose and NaCl.

29. A method for the preparation of the aqueous suspension formulation for aerosol formation comprising lipid or lipidoid nanoparticles which are suspended in an aqueous vehicle solution in accordance with items 1 to 28, said method comprising a step of mixing a solution containing the nucleic acid (a), and a solution containing the ionizable lipid or ionizable lipidoid (b), to form a suspension comprising the lipid or lipidoid nanoparticles;
a step of adding the triblock copolymer which contains one poly(propylene oxide) block and two poly(ethylene oxide) blocks as defined in the preceding items to the suspension; and
a step of subjecting the suspension to tangential flow filtration.

30. The aqueous suspension formulation for aerosol formation in accordance with any of items 1 to 28, which is obtained by the method of item 29.

31. A nebulizer, which comprises a compartment wherein the aqueous suspension formulation for aerosol formation in accordance with any of items 1 to 28 or 30 is contained.

32. The nebulizer in accordance with item 31, which is selected from a jet-nebulizer, a soft mist inhaler and a mesh nebulizer, and which is more preferably a soft mist inhaler or a vibrating mesh nebulizer.

33. An aerosol comprising aerosol droplets dispersed in a gas phase, wherein the aerosol droplets comprise lipid or lipidoid nanoparticles and an aqueous vehicle solution for the nanoparticles,

wherein the lipid or lipidoid nanoparticles comprise the following components (a) and (b):
(a) a nucleic acid and
(b) an ionizable lipid or an ionizable lipidoid;
   and wherein the aqueous vehicle solution comprises a triblock copolymer which contains one poly(propylene oxide) block and two poly(ethylene oxide) blocks.

34. The aerosol in accordance with item 33, wherein the gas phase is air.

35. The aerosol in accordance with item 33 or 34, wherein the mass median aerodynamic diameter (MMAD) of the aerosol droplets ranges from 2 to 10 μm, preferably from 3 to 8 μm.

36. The aerosol in accordance with any of items 33 to 35, wherein the nucleic acid is selected from RNA and plasmid DNA.

37. The aerosol in accordance with any of items 33 to 36, wherein the nucleic acid is selected from mRNA, siRNA, miRNA, antisense RNA, tRNA, and noncoding RNA and is more preferably mRNA.

38. The aerosol in accordance with any of items 33 to 37, wherein the nanoparticles have a Z-average diameter, as determined by dynamic light scattering, in the range of 10 to 500 nm, more preferably in the range of 10 to 250 nm, still more preferably 20 to 200 nm.

39. The aerosol in accordance with any of items 33 to 38, wherein the nanoparticles have a polydispersity index, as determined by dynamic light scattering, in the range of 0.05 to 0.4, more preferably in the range of 0.05 to 0.2.

40. The aerosol in accordance with any of items 33 to 39 wherein the nanoparticles further comprise one or more of the following components (c1) to (c6):
(c1) a non-ionizable lipid having a sterol structure;
(c2) a phosphoglyceride lipid;
(c3) a PEG-conjugated lipid;
(c4) a polysarcosine-conjugated lipid
(c5) a PASylated lipid; and
(c6) a cationic polymer.

41. The aerosol in accordance with any of items 33 to 40, wherein the nanoparticles comprise:
30 to 65 mol % of the ionizable lipid or ionizable lipidoid (b), and one or more of the following components:
10 to 50 mol % of the lipid having a sterol structure (c1),
4 to 50 mol % of the phosphoglyceride lipid (c2),
0.5 to 10 mol % of one of the PEG-conjugated lipid (c3), the polysarcosine-conjugated lipid (c4) and the PASylated lipid (c5), or of any combination thereof,
0.5 to 10 mol % of the cationic polymer (c6),
   such that the sum of (b) and (c1) to (c6) amounts to 100 mol %.

42. The aerosol in accordance with any of items 33 to 39, wherein the nanoparticles further comprise the following components (c1) to (c3):
(c1) a non-ionizable lipid having a sterol structure;
(c2) a phosphoglyceride lipid; and
(c3) a PEG-conjugated lipid.

43. The aerosol in accordance with item 42, wherein the nanoparticles comprise:
30 to 65 mol % of the ionizable lipid or ionizable lipidoid (b),
10 to 50 mol % of the lipid having a sterol structure (c1),
4 to 50 mol % of the phosphoglyceride lipid (c2), and
0.5 to 10 mol % of the PEG-conjugated lipid (c3),
   such that the sum of (b) and (c1) to (c3) amounts to 100 mol %.

44. The aerosol in accordance with any of items 33 to 43, wherein the nanoparticles further comprise a polyanionic component which is different from the nucleic acid.

45. The aerosol in accordance with any of items 33 to 44, wherein the composition of the nanoparticles is such that the weight ratio in the nanoparticles of the sum of the weights of components other than the nucleic acid to the weight of the nucleic acid is in the range of 30:1 to 1:1, more preferably 20:1 to 2:1 and most preferably 15:1 to 3:1.

46. The aerosol in accordance with any of items 33 to 45, wherein the nanoparticles comprise an ionizable lipidoid (b) of the following formula (b-1),

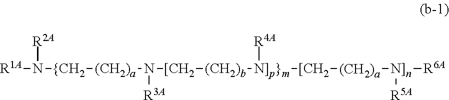

wherein:
a is 1 and b is an integer of 2 to 4; or a is an integer of 2 to 4 and b is 1,
p is 1 or 2,
m is 1 or 2; n is 0 or 1 and m+n is≥2; and
R_{1A} to R_{6A} are independently of each other selected from: hydrogen; —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A}; —CH₂—R_{7A}; —C(NH)—NH₂; a polyethylene glycol) chain; and a receptor ligand; wherein R_{7A} is selected from C3-C18 alkyl and C3-C18 alkenyl having one C—C double bond;
provided that at least two residues among R_{1A} to R_{6A} are selected from —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A} and —CH₂—R_{7A}, wherein R_{7A} is selected from C3-C18 alkyl or C3-C18 alkenyl having one C—C double bond;
   or a protonated form thereof, wherein one or more of the nitrogen atoms contained in the compound of formula (b-1) are protonated to provide a compound carrying a positive charge.

47. The aerosol in accordance with item 46, wherein the nanoparticles comprise an ionizable lipidoid (b) of the following formula (b-1b),

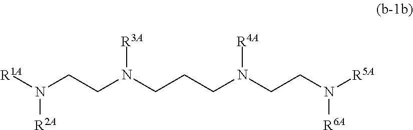

wherein R_{1A} to R_{6A} are defined as in item 46,
   or a protonated form thereof, wherein one or more of the nitrogen atoms contained in the compound of formula (la) are protonated to provide a compound carrying a positive charge.

48. The aerosol in accordance with item 46 or 47, wherein R_{1A} to R_{6A} are independently selected from hydrogen and —CH₂—CH(OH)—R_{7A}, wherein R_{7A} is selected from C8-C18 alkyl and C8-C18 alkenyl having one C—C double bond, provided that at least two residues, preferably at least three residues, and more preferably at least four residues, among R_{1A} to R_{6A} are —CH₂—CH(OH)—R_{7A} wherein R_{7A} is selected from C8-C18 alkyl and C8-C18 alkenyl having one C—C double bond.

49. The aerosol in accordance with any of items 40 to 48, wherein the nanoparticles comprise a non-ionizable lipid having a sterol structure (c1) of formula (c1-1):

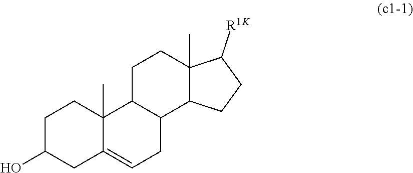

wherein R_{1K} is a C3-C12 alkyl group.

50. The aerosol in accordance with any of items 40 to 49, wherein the non-ionizable lipid having a sterol structure (c1) comprises cholesterol.

51. The aerosol in accordance with any of items 40 to 50, wherein the nanoparticles comprise a phosphoglyceride lipid (c2) of formula (c2-2)

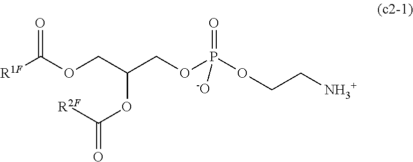

wherein
R_{1F} and R_{2F} are independently a C8-C18 alkyl group or a C8-C18 alkenyl group,
preferably a C12-C18 alkyl group or a C12-C18 alkenyl group,
   or a pharmaceutically acceptable salt thereof;
   or a phosphoglyceride lipid (c2) of formula (c2-2)

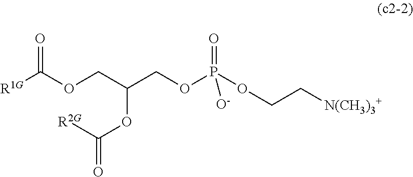

wherein
R_{1G} and R_{2G} are independently a C8-C18 alkyl group or a C8-C18 alkenyl group,
preferably a C12-C18 alkyl group or a C12-C18 alkenyl group,
   or a pharmaceutically acceptable salt thereof.

52. The aerosol in accordance with any of items 40 to 51, wherein the phosphoglyceride lipid (c2) comprises 1,2-dipalmitoyl-sn-glycero-3-phosphocholine (DPPC) or a pharmaceutically acceptable salt thereof.

53. The aerosol in accordance with any of items 40 to 52, wherein the nanoparticles comprise a PEG-conjugated lipid (c3) of formula (c3-1)

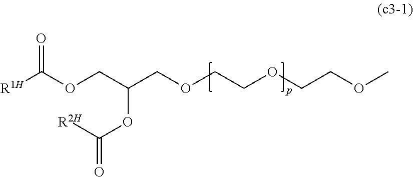

wherein
R_{1H} and R_{2H} are independently a C8-C18 alkyl group or a C8-C18 alkenyl group,
preferably a C12-C18 alkyl group or a C12-C18 alkenyl group, and p is an integer of 5 to 200, preferably 10 to 100, and more preferably 20 to 60;
   or a PEG-conjugated lipid (c3) of formula (c3-2)

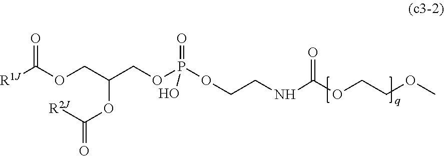

wherein
R_{1J} and R_{2J} are independently a C8-C18 alkyl group or a C8-C18 alkenyl group,
preferably a C12-C18 alkyl group or a C12-C18 alkenyl group, and q is an integer of 5 to 200, preferably 10 to 100, and more preferably 20 to 60,
or a pharmaceutically acceptable salt thereof.

54. The aerosol in accordance with item 53, wherein the PEG conjugated lipid (c3) comprises 1,2-dimyristoyl-sn-glycerolnnethoxy(polyethylene glycol)-2000 (DMG-PEG2k).

55. The aerosol in accordance with any of items 33 to 54, wherein the N/P ratio in the nanoparticles is in the range of 0.5 to 20.

56. The aerosol in accordance with any of items 33 to 55, wherein the triblock copolymer is an A-B-A triblock copolymer which contains one poly(propylene oxide) block B of formula (p-1):

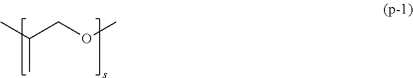

wherein s is an integer of 15 to 67, preferably 20 to 40 and
two poly(ethylene oxides) blocks A of formula (p-2):

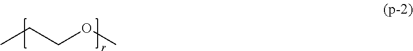

wherein r is, independently for each block, an integer of 2 to 130, preferably 50 to 100, and more preferably 60 to 90.

57. The aerosol in accordance with item 56, wherein the triblock copolymer has the following structure:

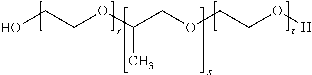

wherein r and t are independently of each other integers of 2 to 130, preferably 50 to 100, and more preferably 60 to 90,
and s is an integer of 15 to 67, preferably 20 to 40.

58. The aerosol in accordance with item 56 or 57, wherein the triblock copolymer is Poloxamer P188.

59. The aerosol in accordance with any of items 33 to 58, wherein the vehicle solution further comprises at least one of sucrose or NaCl, more preferably sucrose and NaCl.

60. The aerosol in accordance with any of items 33 to 59, which is obtainable by nebulization of the aqueous suspension formulation in accordance with any of items 1 to 28 and 30.

61. A method for the preparation of an aerosol, said method comprising a step of nebulizing the aqueous suspension formulation for aerosol formation in accordance with any of items 1 to 28 and 30.

62. The method of item 61, wherein the aerosol is an aerosol in accordance with any of items 33 to 60.

63. The method of item 61 or 62, wherein the nebulization is accomplished by an inhaler selected from a jet-nebulizer, a soft mist inhaler and a mesh nebulizer, more preferably by a soft mist inhaler or a vibrating mesh nebulizer.

64. The aqueous suspension formulation in accordance with any of items 1 to 28 and 30 for use as a medicament, wherein the suspension formulation is to be nebulized and the aerosol provided by nebulization is to be administered to a subject.

65. The aerosol in accordance with any of items 33 to 60 for use as a medicament.

66. The aqueous suspension formulation in accordance with any of items 1 to 28 and 30 for use in the treatment or prevention of a disease or disorder via a nucleic acid-based therapy, wherein the treatment or prevention comprises the nebulization of the aqueous suspension formulation and the administration of the aerosol provided by nebulization to or via the respiratory tract of a subject, preferably via pulmonary administration or nasal administration.

67. The aerosol in accordance with any of items 33 to 60 for use in the treatment or prevention of a disease or disorder via a nucleic acid-based therapy, wherein the treatment or prevention comprises the administration of the aerosol to or via the respiratory tract of a subject, preferably via pulmonary administration or nasal administration.

68. The aqueous suspension formulation for use in accordance with item 66 or the aerosol for use in accordance with item 67, wherein the disease or disorder to be treated or prevented is a pulmonary disease.

69. A method of treatment, comprising the nebulization of the aqueous suspension formulation in accordance with any of items 1 to 28 and 30 and the administration of the aerosol provided by nebulization to or via the respiratory tract of a subject, preferably via pulmonary administration or nasal administration.

70. A method of treatment, comprising the administration of the aerosol in accordance with any of items 33 to 60 to or via the respiratory tract of a subject, preferably via pulmonary administration or nasal administration.

71. The method in accordance with item 69 or 70 for the treatment of a pulmonary disease.

It will be understood that the summary in the above items forms a part of the general disclosure of the present invention, such that the information provided in the following detailed description, e.g. with regard to further preferred embodiments or optional features, also applies for the above items and vice versa.

In the following, a detailed description of the invention will be provided. As will be understood by the skilled person, and unless indicated otherwise in a specific context, the information provided in this context applies to all aspects of the invention, including the aqueous suspension formulation for aerosol formation in accordance with the invention (which may be referred to herein as the “aqueous suspension formulation” or simply as “suspension formulation”), the aerosol in accordance with the invention, and methods and uses involving the suspension formulation or the aerosol.

First, the nanoparticles and their components will be explained. The aqueous suspension formulation for aerosol formation and the aerosol in accordance with the invention comprise lipid nanoparticles or lipidoid nanoparticles. Thus, unless specifically indicated to the contrary, a reference to “nanoparticles” or to “LNPs” herein encompasses the lipid nanoparticles as well as the lipidoid nanoparticles. Moreover, since the aerosol in accordance with the invention can be conveniently prepared using the aqueous suspension formulation, it should be understood that the information provided on the components of the nanoparticles herein applies to the nanoparticles contained in the formulation for aerosol formation in accordance with the invention, and to the nanoparticles contained in the aerosol in accordance with the invention.

As component (a), the nanoparticles contained in the formulation for aerosol formation in accordance with the invention and the nanoparticles contained in the aerosol in accordance with the invention comprise a nucleic acid, which generally provides a pharmaceutically active ingredient of the nanoparticles.

The nature of the nucleic acid is not particularly limited. In principle any type of nucleic acid can be employed in the context of the present invention. Nucleic acids are known to the skilled person and refer to biopolymers or small biomolecules composed of nucleotides which are the monomers made of three components: a 5-carbon sugar, a phosphate group and a nitrogenous base.

The term nucleic acid is the overall name for DNA (deoxyribonucleic acid) and RNA (ribonucleic acid), i.e., the members of the above family of biopolymers. If the sugar is a compound ribose, the polymer is RNA if the sugar is derived from ribose as deoxyribose, the polymer is DNA. The term “nucleic acid” encompasses oligonucleotides or polynucleotides. As a nucleic acid is a biopolymer composed of nucleotides, the term “nucleic acid” is also often referred to as a “sequence of nucleotides” and, accordingly, as will be understood by the skilled person, the terms “nucleic acid” and “nucleic acid sequence” are often used interchangeably.

In a preferred embodiment, the nanoparticles of the aqueous suspension formulation in accordance with the invention and of the aerosol in accordance with the invention comprise ribonucleic acid (RNA) as nucleic acid, more preferably single stranded RNA, and most preferred is mRNA.

The term “nucleic acid” encompasses all forms of naturally occurring types of nucleic acids as well as chemically and/or enzymatically synthesized nucleic acids and also encompasses nucleic acid analogues and nucleic acid derivatives. The term in particular includes any backbone-modified, sugar-modified or base-modified single-stranded or double-stranded nucleic acid, such as e.g. locked nucleic acids (LNA), peptide nucleic acids (PNA), oligonucleoside thiophosphates and phosphotriesters, morpholino oligonucleotides, cationic oligonucleotides (U.S. Pat. No. 6,017,700 A, WO/2007/069092), substituted ribo-oligonucleotides or phosphorothioates. Furthermore, the term “nucleic acid” also refers to any molecule that comprises nucleotides or nucleotide analogues. There are no limitations concerning sequence or size of a nucleic acid comprised in the nanoparticles of the present invention. The nucleic acid is predominantly defined by the biological effect that is to be achieved at the biological target the nanoparticles of the present invention are delivered to. For instance, as will be outlined in more detail further below, in the case of an application in gene or nucleic acid therapy, the nucleic acid or nucleic acid sequence can be defined by the gene or gene fragment that is to be expressed or by the intended substitution or repair of a defective gene or any gene target sequence or by the target sequence of a gene to be inhibited, knocked-down, down- regulated or up-regulated.

The nanoparticles of the suspension and the aerosol in accordance with the invention may comprise a nucleic acid being a DNA molecule. A preferred embodiment of such a DNA molecule is a DNA molecule which can be transcribed into an mRNA molecule. Transcription is the first step of gene expression, in which a particular segment of a DNA molecule is copied into an mRNA molecule by the enzyme RNA polymerase. During transcription, a DNA sequence is read by an RNA polymerase, which produces a complementary, anti-parallel RNA strand called a primary transcript.

A DNA molecule according to the present invention may be introduced in a vector, preferably an expression vector, by standard molecular biology techniques (see, e.g. Sambrook et al., Molecular Cloning, A laboratory manual, 2nd Ed, 1989). The term “vector” such as “expression vector” or “cloning vector” in the sense of the present invention is understood as a circular, double-stranded unit of DNA that is preferably able to replicate within a cell independently of the chromosomal DNA and which is used as a vehicle to carry genetic material into a cell, where it can be (replicated and/or) expressed (i.e., transcribed into RNA and translated into a amino acid sequence). A vector containing foreign DNA is termed recombinant DNA. The vector itself is generally a DNA sequence that typically consists of an insert (e.g., a nucleic acid molecule/DNA molecule of the present invention) and a larger sequence that serves as the “backbone” of the vector. Plasmids in the sense of the present invention are most often found in bacteria and are used in recombinant DNA research to transfer genes between cells and are as such a subpopulation of “vectors” as used in the sense of the present invention.

It is evident to the person skilled in the art that further regulatory sequences may be added to the DNA molecule of the invention. For example, transcriptional enhancers and/or sequences which allow for induced expression may be employed. A suitable inducible system is for example tetracycline-regulated gene expression as described, e.g., by Gossen and Bujard, Proc. Natl. Acad. Sci. USA 89 (1992), 5547-5551) and Gossen, Trends Biotech. 12 (1994), 58-62, or a dexamethasone-inducible gene expression system as described, e.g. by Crook, EMBO J. 8 (1989), 513-519. The present invention may also use a vector, preferably an expression vector, comprising the DNA molecule. The vector may be, e.g., a plasmid, cosmid, virus, bacteriophage or another vector used e.g. conventionally in genetic engineering, and may comprise further genes such as marker genes which allow for the selection of said vector in a suitable host cell and under suitable conditions.

If the nucleic acid used in the context of the present invention is a DNA molecule, it can be a plasmid DNA (pDNA) molecule.

As noted above, the nanoparticles of the aqueous suspension formulation in accordance with the invention and of the aerosol in accordance with the invention preferably comprise ribonucleic acid (RNA) as nucleic acid, more preferably single stranded RNA, and most preferred is mRNA.

As regards RNA, in principle any type of RNA can be employed in the context of the present invention. In a preferred embodiment the RNA is a single-stranded RNA. The term “single-stranded RNA” means a single consecutive chain of ribonucleotides in contrast to RNA molecules in which two or more separate chains form a double-stranded molecule due to hybridization of the separate chains. The term “single-stranded RNA” does not exclude that the single-stranded molecule forms in itself double-stranded structures such as secondary (e.g. loops and stem-loops) or tertiary structures. Examples are tRNA and mRNA but also any other type of single-stranded RNA like antisense-RNA, siRNA and the like.

The term “RNA” covers RNA which codes for an amino acid sequence as well as RNA which does not code for an amino acid sequence. It has been suggested that more than 80% of the genome contains functional DNA elements that do not code for proteins. These noncoding sequences include regulatory DNA elements (binding sites for transcription factors, regulators and coregulators etc.) and sequences that code for transcripts that are never translated into proteins. These transcripts, which are encoded by the genome and transcribed into RNA but do not get translated into proteins, are called noncoding RNAs (ncRNAs). Thus, in one embodiment the RNA is a noncoding RNA. Preferably, the noncoding RNA is a single-stranded molecule. Studies demonstrate that ncRNAs are critical players in gene regulation, maintenance of genomic integrity, cell differentiation, and development, and they are misregulated in various human diseases. There are different types of ncRNAs: short (20-50 nt), medium (50-200 nt), and long (>200 nt) ncRNAs. Short ncRNA includes microRNA (miRNA), small interfering RNA (siRNA), piwi-interacting RNA (piRNA), and transcription initiating RNA (tiRNA). Examples of medium ncRNAs are small nuclear RNAs (snRNAs), small nucleolar RNAs (snoRNAs), transfer RNAs (tRNAs), transcription start-site-associated RNAs (TSSaRNAs), promoter-associated small RNAs (PASRs), and promoter upstream transcripts (PROMPTs). Long noncoding RNAs (IncRNA) include long-intergenic noncoding RNA (lincRNA), antisense-IncRNA, intronic IncRNA, transcribed ultra-conserved RNAs (T-UCRs), and others (Bhan A, Mandal S S, ChemMedChem. 2014 Mar. 26. doi:10.1002/cmdc.201300534). Of the above-mentioned non-coding RNAs only siRNA is double-stranded. Thus, since in a preferred embodiment the noncoding RNA is single-stranded, it is preferred that the noncoding RNA is not siRNA. In another embodiment the RNA is a coding RNA, i.e. an RNA which codes for an amino acid sequence. Such RNA molecules are also referred to as mRNA (messenger RNA) and are single-stranded RNA molecules. The RNA may be made by synthetic chemical and enzymatic methodology known to one of ordinary skill in the art, or by the use of recombinant technology, or may be isolated from natural sources, or by a combination thereof.

Messenger RNAs (mRNA) are copolymers which are built up of nucleoside phosphate building blocks mainly with adenosine, cytidine, uridine and guanosine as nucleosides, which as intermediate carriers bring the genetic information from the DNA in the cell nucleus into the cytoplasm, where it is translated into proteins. They are thus suitable as alternatives for gene expression.

In the context of the present invention, mRNA should be understood to mean any polyribonucleotide molecule which, if it comes into the cell, is suitable for the expression of a protein or fragment thereof or is translatable to a protein or fragment thereof. The term “protein” here encompasses any kind of amino acid sequence, i.e. chains of two or more amino acids which are each linked via peptide bonds and also includes peptides and fusion proteins.

The mRNA contains a ribonucleotide sequence which encodes a protein or fragment thereof whose function in the cell or in the vicinity of the cell is needed or beneficial, e.g. a protein the lack or defective form of which is a trigger for a disease or an illness, the provision of which can moderate or prevent a disease or disorder, or a protein which can promote a process which is beneficial for the body, in a cell or its vicinity. The mRNA may contain the sequence for the complete protein or a functional variant thereof. Further, the ribonucleotide sequence can encode a protein which acts as a factor, inducer, regulator, stimulator or enzyme, or a functional fragment thereof, where this protein is one whose function is necessary in order to remedy a disorder, in particular a metabolic disorder or in order to initiate processes in vivo such as the formation of new blood vessels, tissues, etc. Examples for proteins which can be encoded by mRNA include antibodies, cytokines or chemokines. Here, functional variant is understood to mean a fragment which in the cell can undertake the function of the protein whose function in the cell is needed or the lack or defective form whereof is pathogenic. In addition, the mRNA may also have further functional regions and/or 3′ or 5′ noncoding regions, in particular 3′ and/or 5′ UTRs. The 3′ and/or 5′ noncoding regions can be the regions naturally flanking the protein-encoding sequence or artificial sequences, e.g. sequences which contribute to the stabilization of the RNA. Those skilled in the art can determine the sequences suitable for this in each case by routine experiments.

In a preferred embodiment, the mRNA contains a 5′-cap (five-prime-cap; cap-0) consisting of a m7GpppG connected to the mRNA via a 5′ to 5′ triphosphate linkage, an additional methyl group onto the penultimate nucleotide from the 5′-end of the mRNA (Cap-1, Anti-Reverse Cap Analog (ARCA)) and/or an internal ribosome entry site (IRES) and/or a polyA-tail at the 3′-end, in particular, in order to improve translation. The mRNA can have further regions promoting translation such as, for example, cap-2 structures or histone stem-loop structures.

The RNA which may be present in the suspension formulation and the aerosol according to the present invention may contain unmodified and modified nucleotides. The term “unmodified nucleotide” used herein refers to A, C, G and U nucleotides. The term “modified nucleotide” used herein refers to any naturally occurring or non-naturally occurring isomers of A, C, G and U nucleotides as well as to any naturally occurring or naturally occurring analogues, alternative or modified nucleotide or isomer thereof having for example chemical modifications or substituted residues. Modified nucleotides can have a base modification and/or a sugar modification. Modified nucleotides can also have phosphate group modifications, e.g., with respect to the 5′-prime cap of an mRNA molecule. Modified nucleotides also include nucleotides that are synthesized post-transcriptionally by covalent modification of the nucleotides. Further, any suitable mixture of non-modified and modified nucleotides is possible. A non-limiting number of examples of modified nucleotides can be found in the literature (e.g. US 2013/0123481 A1; Cantara et al., Nucleic Acids Res, 2011, 39(Issue suppl_1):D195-D201; Helm and Alfonzo, Chem Biol, 2014, 21(2):174-185; or Carell et al., Angew Chem Int Ed Engl, 2012, 51(29):7110-31) and some preferable modified nucleotides are mentioned exemplarily in the following based on their respective nucleoside residue: 1-methyladenosine, 2-methylthio-N6-hydroxynorvalyl carbamoyladenosine, 2-methyladenosine, 2′-O-ribosylphosphate adenosine, N6-methyl-N6-threonylcarbamoyladenosine, N6-acetyladenosine, N6-glycinylcarbamoyladenosine, N6-isopentenyladenosine, N6-methyladenosine, N6-threonylcarbamoyladenosine, N6,N6-dimethyladenosine, N6-(cis-hydroxyisopentenyl)adenosine, N6-hydroxynorvalylcarbamoyladenosine, 1,2′-O-dimethyladenosine, N6,2′-O-dimethyladenosine, 2′-O-methyladenosine, N6,N6,2′-O-trimethyladenosine, 2-methylthio-N6-(cis-hydroxyisopentenyl)adenosine, 2-methylthio-N6-methyladenosine, 2-methylthio-N6-isopentenyladenosine, 2-methylthio-N6-threonyl carbamoyladenosine, N6-2-methylthio-N6-threonyl carbamoyladenosine, 2-methylthio-N6-(cis-hydroxyisopentenyl)adenosine, 7-methyladenosine, 2-methylthio-adenosine, 2-methoxy-adenosine, 2′-amino-2′-deoxyadenosine, 2′-azido-2′-deoxyadenosine, 2′-fluoro-2′-deoxyadenosine, 2-aminopurine, 2,6-diaminopurine, 7-deaza-adenosine, 7-deaza-8-aza-adenosine, 7-deaza-2-aminopurine, 7-deaza-8-aza-2-aminopurine, 7-deaza-2,6-diaminopurine, 7-deaza-8-aza-2,6-diaminopurine; 2-thiocytidine, 3-methylcytidine, N4-acetylcytidine, 5-formylcytidine, N4-methylcytidine, 5-methylcytidine, 5-hydroxymethylcytidine, 5-hydroxycytidine, lysidine, N4-acetyl-2′-O-methylcytidine, 5-formyl-2′-O-methylcytidine, 5,2′-O-dimethylcytidine, 2-O-methylcytidine, N4,2′-O-dimethylcytidine, N4,N4,2′-O-trimethylcytidine, isocytidine, pseudocytidine, pseudoisocytidine, 2-thio-cytidine, 2′-methyl-2′-deoxycytidine, 2′-amino-2′-deoxycytidine, 2′-fluoro-2′-deoxycytidine, 5-iodocytidine, 5-bromocytidine, 2′-azido-2′-deoxycytidine, 2′-amino-2′-deoxycytidine, 2′-fluor-2′-deoxycytidine, 5-aza-cytidine, 3-methyl-cytidine, 1-methyl-pseudoisocytidine, pyrrolo-cytidine, pyrrolo-pseudoisocytidine, 2-thio-5-methyl-cytidine, 4-thio-pseudoisocytidine, 4-thio-l-methyl-pseudoisocytidine, 4-thio-l-methyl-1-deaza-pseudoisocytidine, 1-methyl-l-deaza-pseudoisocytidine, 2-methoxy-cytidine, 2-methoxy-5-methyl-cytidine, 4-methoxy-pseudoisocytidine, 4-methoxy-l-methyl-pseudoisocytidine, zebularine,5-aza-zebularine, 5-methyl-zebularine, 5-aza-2-thio-zebularine, 2-thio-zebularine; 1-methylguanosine, N2,7-dimethylguanosine, N2-methylguanosine, 2′-O-ribosylphosphate guanosine, 7-methylguanosine, hydroxywybutosine, 7-aminomethyl-7-deazaguanosine, 7-cyano-7-deazaguanosine, N2,N2-dimethylguanosine, N2,7,2′-O-trimethylguanosine, N2,2′-O-dimethylguanosine, 1,2′-O-dimethylguanosine, 2′-O-methylguanosine, N2,N2,2′-O-trimethylguanosine, N2,N2J-trimethylguanosine, Isoguanosine, 4-demethylwyosine, epoxyqueuosine, undermodified hydroxywybutosine, methylated undermodified hydroxywybutosine, isowyosine, peroxywybutosine, galactosyl-queuosine, mannosyl-queuosine, queuosine, archaeosine, wybutosine, methylwyosine, wyosine, 7-aminocarboxypropyldemethylwyosine, 7-aminocarboxypropylwyosine, 7-aminocarboxypropylwyosinemethylester, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, N1-methylguanosine, 2′-amino-3′-deoxyguanosine, 2′-azido-2′-deoxyguanosine, 2′-fluoro-2′-deoxyguanosine, 2-thiouridine, 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine, 5-(carboxyhydroxymethyl)-2′-O-methyluridine methyl ester, 5-(isopentenylaminomethyl)uridine, 5-(isopentenylaminomethyl)-2-thiouridine, 3,2′-O-dimethyluridine, 5-carboxymethylaminomethyl-2′-O-methyluridine, 5-carbamoylhydroxymethyluridine, 5-carbamoylmethyl-2′-O-methyluridine, 5-carbamoylmethyl-2-thiouridine, 5-methoxycarbonylmethyl-2′-O-methyluridine, 5-(isopentenylaminomethyl)-2′-O-methyluridine, 5,2′-O-dimethyluridine, 2′-O-methyluridine, 2′-O-methyl-2-thiorudine, 2-thio-2′-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl)pseudouridine, 1-methylpseudouridine, 3-methylpseudouridine, 2′-O-methylpseudouridine, 5-formyluridine, 5-aminomethyl-2-geranyluridine, 5-taurinomethyluridine, 5-iodouridine, 5-bromouridine, 2′-methyl-2′-deoxyuridine, 2′-amino-2′-deoxyuridine, 2′-azido-2′-deoxyuridine, 2′-fluoro-2′-deoxyuridine, inosine, 1-methylinosine, 1,2′-O-dimethylinosine, 2′-O-methylinosine, 5-aza-uridine, 2-thio-5-aza-uridine, 4-thio-pseudouridine, 2-thio-pseudouridine, 5-carboxymethyl-uridine, 1-carboxymethyl-pseudouridine, 5-propynyl-uridine, 1-propynyl-pseudouridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-l-deaza-pseudouridine, 2-thio-1-methyl-l-deaza-pseudouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 1,2′-O-dimethyladenosine, 1,2′-O-dimethylguanosine, 1,2′-O-dimethylinosine, 2,8-dimethyladenosine, 2-methylthiomethylenethio-N6-isopentenyl-adenosine, 2-geranylthiouridine, 2-lysidine, 2-methylthio cyclic N6-threonylcarbamoyladenosine, 2-methylthio-N6-(cis-hydroxyisopentenyl) adenosine, 2-methylthio-N6-hydroxynorvalylcarbamoyladenosine, 2-methylthio-N6-threonylcarbamoyladenosine, 2-selenouridine, 2-thio-2′-O-methyluridine, 2′-O-methyladenosine, 2′-O-methylcytidine, 2′-O-methylguanosine, 2′-O-methylinosine, 2′-O-methylpseudouridine, 2′-O-methyluridine, 2′-O-methyluridine 5-oxyacetic acid methyl ester, 2′-O-ribosyladenosinephosphate, 2′-O-ribosylguanosinephosphate, 3,2′-O-dimethyluridine, 3-(3-amino-3-carboxypropyl)-5,6-dihydrouridine, 3-(3-amino-3-carboxypropyl)pseudouridine, 5,2′-O-dimethylcytidine, 5,2′-O-dimethyluridine, 5-(carboxyhydroxymethyl)-2′-O-methyluridine methyl ester, 55-(isopentenylaminomethyl)-2′-O-methyluridine, 5-aminomethyl-2-geranylthiouridine, 5-aminomethyl-2-selenouridine, 5-aminomethyluridine, 5-carbamoylmethyl-2′-O-methyluridine, 5-carboxyhyd roxymethylu ridine, 5-carboxymethyl-2-thiouridine, 5-carboxymethylaminomethyl-2-geranylthiouridine, 5-carboxymethylaminomethyl-2-selenouridine, 5-carboxymethylaminomethyl-2′-O-methyluridine, 5-cyanomethyluridine, 5-formyl-2′-O-methylcytidine, 5-methoxycarbonylmethyl-2′-O-methyluridine, 5-methylaminomethyl-2-geranylthiouridine, 7-aminocarboxypropyl-demethylwyosine, 7-methylguanosine, 8-methyladenosine, N2,2′-O-dimethylguanosine, N2,7,2′-O-trimethylguanosine, N2,7-dimethylguanosine, N2,N2,2′-O-trimethylguanosine, N2,N2,7-trimethylguanosine, N2,N2,7-trimethylguanosine, N4,2′-O-dimethylcytidine, N4,N4,2′-O-trimethylcytidine, N4,N4-dimethylcytidine, N4-acetyl-2′-O-methylcytidine, N6,2′-O-dimethyladenosine, N6,N6,2′-O-trimethyladenosine, N6-formyladenosine, N6-hydroxymethyladenosine, agmatidine, 2-methylthio cyclic N6-threonylcarbamoyladenosine, glutamyl-queuosine, guanosine added to any nucleotide, guanylylated 5′ end, hydroxy-N6-threonylcarbamoyladenosine; most preferably pseudo-uridine, N1-methyl-pseudo-uridine, 2′-fluoro-2′-deoxycytidine, 5-iodocytidine, 5-methylcytidine, 2-thiouridine, 5-iodouridine and/or 5-methyl-uridine.

Furthermore, the term “modified nucleotide” comprises nucleotides containing isotopes such as deuterium. The term “isotope” refers to an element having the same number of protons but different number of neutrons resulting in different mass numbers. Thus, isotopes of hydrogen for example are not limited to deuterium, but include also tritium. Furthermore, the polyribonucleotide can also contain isotopes of other elements including for example carbon, oxygen, nitrogen and phosphor. It is also possible that modified nucleotides are deuterated or contain another isotope of hydrogen or of oxygen, carbon, nitrogen or phosphorus.

Among the U, C, A and G nucleotides either none, one, two, three or all of them can be modified. Hence, in some embodiments, at least one nucleotide of one nucleotide type, e.g. at least one U nucleotide, can be a modified nucleotide. In some embodiments, at least one nucleotide of in total two nucleotide types, e.g. at least one U nucleotide and at least one C nucleotide, can be a modified nucleotide. In some embodiments, at least one nucleotide of in total three nucleotide types, e.g. at least one G nucleotide, at least one U nucleotide and at least one C nucleotide, can be a modified nucleotide. In some embodiments, at least one nucleotide of all four nucleotide types can be a modified nucleotide. In all these embodiments one or more nucleotides per nucleotide type can be modified, the percentage of said modified nucleotides of per nucleotide type being 0%, 2.5%, 5%, 7.5%, 10%, 15%, 20%, 25%, 30%, 35%, 40%, 45%, 50%, 55%, 60%, 65%, 70%, 75%, 80%, 85%, 90% or 100%.

In some embodiments, the total percentage of modified nucleotides comprised in the mRNA molecules is 0%, 2.5%, 5%, 7.5%, 10%, 15%, 20%, 25%, 30%, 35%, 40%, 45%, 50%, 55%, 60%, 65%, 70%, 75%, 80%, 85%, 90% or 100%.

In a preferred embodiment the mRNA is an mRNA which contains a combination of modified and unmodified nucleotides. Preferably, it is an mRNA containing a combination of modified and unmodified nucleotides as described in WO2011/012316. The mRNA described therein is reported to show an increased stability and diminished immunogenicity. In a preferred embodiment, in such a modified mRNA 5 to 50% of the cytidine nucleotides and 5 to 50% of the uridine nucleotides are modified. The adenosine- and guanosine-containing nucleotides can be unmodified. The adenosine and guanosine nucleotides can be unmodified or partially modified, and they are preferably present in unmodified form.

In certain embodiments of any of the foregoing, the percentage of analogues of a given nucleotide refers to input percentage (e.g., the percentage of analogues in a starting reaction, such as a starting in vitro transcription reaction). In certain embodiments of any of the foregoing, the percentage of analogues of a given nucleotide refers to output (e.g., the percentage in a synthesized or transcribed compound). Both options are equally contemplated.

The RNA, preferably the mRNA, molecules of the present invention may be produced recombinantly in in vivo systems by methods known to a person skilled in the art.

Alternatively, the modified RNA, preferably the mRNA molecules of the present invention may be produced in an in vitro system using, for example, an in vitro transcription system which is known to the person skilled in the art. An in vitro transcription system capable of producing RNA, preferably mRNA requires an input mixture of modified and unmodified nucleoside triphosphates to produce modified RNA, preferably mRNA molecules with the desired properties of the present invention. In certain embodiments, 5 to 50% of the cytidines are analogues of cytidine in such an input mixture and 5 to 50% of the uridines are analogues of uridine in such an input mixture. In certain embodiments, 5 to 40% of the cytidines are analogues of cytidine in such an input mixture and 5 to 40% of the uridines are analogues of uridine in such an input mixture. In certain embodiments, 5 to 30% of the cytidines are analogues of cytidine in such a mixture and 5 to 30% of the uridines are analogues of uridine in such an input mixture. In certain embodiments, 5 to 30% of the cytidines are analogues of cytidine in such mixture and 10 to 30% of the uridines are analogues of uridine in such mixture. In certain embodiments, 5 to 20% of the cytidines are analogues of cytidine in such an input mixture and 5 to 20% of the uridines are analogues of uridine in such an input mixture. In certain embodiments, 5 to 10% of the cytidines are analogues of cytidine in such an input mixture and 5 to 10% of the uridines are analogues of uridine in such an input mixture. In certain embodiments, 25% of the cytidines are analogues of cytidine in such an input mixture and 25% of the uridines are analogues of uridine in such an input mixture. In certain embodiments, the input mixture does not comprise analogues of adenosine and/or guanosine. In other embodiments, optionally, the input mixture comprises one or more analogues of adenosine and/or guanosine (or none of either or both).

In certain embodiments, the percentage of cytidines in an input mixture that are analogues of cytidine is not the same as the percentage of uridines in an input mixture that are analogues of uridine. In certain embodiments, the percentage of analogues of cytidine in an input mixture is lower than the percentage of analogues of uridine in an input mixture. As noted above, this may be in the presence or the absence of analogues of adenosine and guanosine in the input mixture but, in certain embodiments, is in the absence of analogues of adenosine and analogues of guanosine in the input mixture.

In certain embodiments, an input mixture of nucleotides for an in vitro transcription system that produces a RNA, preferably mRNA of the present invention comprises analogues of cytidine and analogues of uridine, and 5 to 20% of the cytidines of the input mixture are analogues of cytidine and 25 to 45% of the uridines of the input mixture are analogues of uridine. In other words, the input mixture comprises modified and unmodified cytidines and modified and unmodified uridines, and 5 to 20% of the cytidines of the input mixture comprise analogues of cytidine while 25 to 45% of the uridines of the input mixture comprise analogues of uridine. In other embodiments, the input mixture comprises 5 to 10% analogues of cytidine and 30 to 40% analogues of uridine, such as 7-9% analogues of cytidine, such as 7, 7.5 or 8% and, such as 32-38% analogues of uridine, such as 33, 34, 35, 36%.

In certain embodiments, any of the analogues of uridine and analogues of cytidine described herein may be used, optionally excluding pseudouridine. In certain embodiments, the analogue of cytidine comprises or consists of (e.g., it is the single C analogue type used) 5-iodocytidine and the analogue of uridine comprises or consists of (e.g., it is the single U analogue type used) 5-iodouridine.

Exemplary analogues are described above. It should be understood that for modified polyribonucleotides encoding the desired polypeptide, the analogues and level of modification is, unless indicated otherwise, considered across the entire polyribonucleotide encoding the desired polypeptide, including 5′ and 3′ untranslated regions (e.g., the level of modification is based on input ratios of analogues in an in vitro transcription reaction such that analogues may be incorporated at positions that are transcribed).

Furthermore, the modified RNA, preferably mRNA molecules may be chemically synthesized, e.g., by conventional chemical synthesis on an automated nucleotide sequence synthesizer using a solid-phase support and standard techniques or by chemical synthesis of the respective DNA sequences and subsequent in vitro or in vivo transcription of the same.

In another preferred embodiment, the mRNA may be combined with target binding sites, targeting sequences and/or with micro-RNA binding sites, in order to allow activity of the desired mRNA only in the relevant cells. In a further preferred embodiment, the RNA can be combined with micro-RNAs or shRNAs in the untranslated regions.

In general, therapeutic effects can be achieved by the interaction of the ribonucleic acid with cellular molecules and organelles. Such interaction alone may for example activate the innate immune system, as is the case for certain CpG oligonucleotides and sequences designed to specifically interact with toll-like and other extra- or intracellular receptors. Furthermore, the uptake or introduction of nucleic acids (preferably ribonucleic acids, more preferably mRNAs) in cells can be intended to lead to the expression of nucleotide sequences such as genes comprised in the nucleic acid (preferably ribonucleic acids, more preferably the mRNA), can be intended for the downregulation, silencing or knockdown of endogenous gene expression as a consequence of the intracellular presence of an introduced exogenous nucleic acid, or can be intended for the modification of endogenous nucleic acid sequences such as repair, excision, insertion or exchange of selected bases or of whole stretches of endogenous nucleic acid sequences, or can be intended for interference with virtually any cellular process as a consequence of the intracellular presence and interaction of an introduced exogenous ribonucleic acid (preferably an mRNA). Overexpression of introduced exogenous nucleic acids (preferably ribonucleic acids, more preferably mRNAs) may be intended to compensate or complement endogenous gene expression, in particular in cases where an endogenous gene is defective or silent, leading to no, insufficient or a defective or a dysfunctional product of gene expression such as is the case with many metabolic and hereditary diseases like cystic fibrosis, hemophilia or muscular dystrophy to name a few. Overexpression of introduced exogenous nucleic acids (preferably ribonucleic acids, more preferably mRNAs) may also be intended to have the product of the expression interact or interfere with any endogenous cellular process such as the regulation of gene expression, signal transduction and other cellular processes. The overexpression of introduced exogenous nucleic acids (preferably ribonucleic acids, more preferably mRNAs) may also be intended to give rise to an immune response in context of the organism in which a transfected or transduced cell resides or is made to reside. Examples are the genetic modification of antigen-presenting cells such as dendritic cells in order to have them present an antigen for vaccination purposes. Other examples are the overexpression of cytokines in tumors in order to elicit a tumor-specific immune response. Furthermore, the overexpression of introduced exogenous ribonucleic acids (preferably mRNAs) may also be intended to generate in vivo or ex vivo transiently genetically modified cells for cellular therapies such as modified T-cells, NK cells and other lymphocytes or precursor or stem or other cells for regenerative medicine.

Downregulation, silencing or knockdown of endogenous gene expression for therapeutic purposes can for example be achieved by RNA interference (RNAi), with ribozymes, antisense oligonucleotides, tRNAs, long double-stranded RNA where such downregulation can be sequence-specific or unspecific and can also lead to cell death as is the case when long double-stranded RNAs are introduced into cells. Downregulation, silencing or knockdown of endogenous or pre-existing gene expression can be useful in the treatment of acquired, hereditary or spontaneously incurring diseases including viral infections and cancer. It can also be envisaged that the introduction of nucleic acids into cells can be practiced as a preventive measure in order to prevent, for example, viral infection or neoplasias. Downregulation, silencing or knockdown of endogenous gene expression can be exerted on the transcriptional level and on the translational level. Multiple mechanisms are known to the one skilled in the art and include for example epigenetic modifications, changes in chromatin structure, selective binding of transcription factors by the introduced nucleic acid, hybridization of the introduced nucleic acid to complementary sequences in genomic DNA, mRNA or other RNA species by base pairing including unconventional base pairing mechanisms such as triple helix formation. Similarly, gene repair, base or sequence changes can be achieved at the genomic level and at the mRNA level including exon skipping. Base or sequence changes can for example be achieved by RNA-guided site-specific DNA cleavage, by cut and paste mechanisms exploiting trans-splicing, trans-splicing ribozymes, chimeraplasts, splicosome-mediated RNA trans-splicing, or by exploiting group II or retargeted introns, or by exploiting insertional mutagenesis mediated by viruses or exploiting targeted genomic insertion using prokaryotic, eukaryotic or viral integrase systems. As nucleic acids are the carriers of the building plans of living systems and as they participate in many cellular processes in a direct and indirect manner, in theory any cellular process can be influenced by the introduction of nucleic acids into cells from outside. Notably, this introduction can be carried out directly in vivo and ex vivo in cell or organ culture followed by transplantation of thus modified organs or cells into a recipient. The particles for use in the context of the present invention with nucleic acids as therapeutically active agent may be useful for all purposes described above.

As mentioned above, the RNA, preferably the mRNA, may contain a ribonucleotide sequence which encodes a protein or fragment thereof whose function in the cell or in the vicinity of the cell is needed or beneficial, e.g. a protein the lack or defective form of which is a trigger for a disease or an illness, the provision of which can moderate or prevent a disease or an illness, or a protein which can promote a process which is beneficial for the body, in a cell or its vicinity.

Indeed, in recent years, RNA (in particular, mRNA) has become increasingly relevant as a new drug entity. As opposed to DNA-based gene therapeutics, mRNA does not need to be transported into the nucleus but is directly translated into protein in the cytoplasm (J Control Release, 2011, 150:238-247, and Eur J Pharm Biopharm, 2009, 71:484-489).

Moreover, numerous genetic disorders, caused by the mutation of a single gene are known and candidates for RNA, preferably mRNA, therapeutic approaches. Disorders caused by single-gene mutations, like cystic fibrosis, hemophilia and many others, can be dominant or recessive with respect to the likelihood that a certain trait will appear in the offspring. While a dominant allele manifests a phenotype in individuals who have only one copy of the allele, for a recessive allele the individual must have two copies, one from each parent to become manifest. In contrast, polygenic disorders are caused by two or more genes and the manifestation of the respective disease is often fluent and associated to environmental factors. Examples for polygenic disorders are hypertension, elevated cholesterol level, cancer, neurodegenerative disorders, mental illness and others. Also in these cases therapeutic RNA, preferably the mRNA, representing one or more of these genes may be beneficial to those subjects. Furthermore, a genetic disorder must not have been passed down from the parents' genes, but can also be caused by new mutations. Also in these cases therapeutic RNA, preferably the mRNA, representing the correct gene sequence may be beneficial to the subjects.

An online catalog with presently 22,993 entries of Human Genes and Genetic Disorders together with their respective genes and a description of their phenotypes are available at the ONIM (Online Mendelian Inheritance in Man) webpage (http://onim.org); sequences of each are available from the Uniprot database (http://www.uniprot.org). As non-limiting examples, the following Table A lists some congenital diseases and disorders, and the corresponding gene(s). Due to the high degree of interaction of cellular signaling pathways, the mutation of a certain gene causes a multiply of pathogenic symptoms, of which only a characteristic one is listed in Table A.

In some embodiments of the present invention, the therapeutic protein which is encoded by the RNA, preferably the mRNA, which may be present in the suspension formulation and the aerosol of the present invention is chosen from the cellular proteins listed in Table A. Thus, the RNA, preferably the mRNA, molecule may encode a therapeutic cellular protein, wherein the encoded therapeutic protein is one listed in Table A or a homolog thereof.

In another embodiment of the present invention, the therapeutic protein which is encoded by the RNA, preferably the mRNA, is chosen from the secreted proteins listed in Table A. Thus, the RNA, preferably the mRNA, may encode a therapeutic fusion protein, wherein the encoded therapeutic protein or a homolog thereof is one listed in Table A and the second protein is a signal peptide that allows the secretion of the therapeutic protein. A signal peptide is a short, typically 5-30 amino acids long sequence present at the N-terminus of said therapeutic protein and that leads the fusion protein towards the cell's secretory pathway via certain organelles (i.e. the endoplasmic reticulum, the golgi-apparatus or the endosomes). Thus, such fusion protein is secreted from the cell or from a cellular organelle or inserted into a cellular membrane (e.g. multi-spanning trans- membrane proteins) at a cellular compartment or at the cell's surface.

Thus, in preferred embodiments of the present invention the RNA, preferably the mRNA, may encode one or more, but is not limited to, the following proteins of the genes that cause, predispose or protect from diseases. Non-limiting examples of such diseases or disorders that may be treated (or prevented) include those wherein said polypeptide, protein or peptide is selected from the group consisting of the ones as outlined in the following Table A.

In some embodiments, the encoding sequence of the RNA, preferably the mRNA, may be transcribed and translated into a partial or full-length protein comprising cellular activity at a level equal to or greater than that of the native protein. In some embodiments, the RNA, preferably the mRNA, encodes a therapeutically or pharmaceutically active polypeptide, protein or peptide having a therapeutic or preventive effect, wherein said polypeptide, protein or peptide is selected from the group consisting of the ones as outlined in the following Table A. The RNA, preferably the mRNA, more specifically the encoding sequence thereof, may be used to express a partial or full-length protein with cellular activity at a level equal to or less than that of the native protein. This may allow the treatment of diseases for which the administration of an RNA molecule can be indicated.

TABLE A
Non-limiting examples of human genes and genetic diseases or disorders

| | | |
|---|---|---|
| Disease | Pathology | Gene, heredity |

Blood diseases

| | | |
|---|---|---|
| Fanconi Anemia | Anemia and | FANCA, autosomal |
| | neutropenia, evidence | recessive |
| | that a DNA repair | |
| | mechanism is affected | |
| Hemophilia-A | Abnormal bleeding | Coagulation Factor VIII, X- |
| | | chromosomal recessive |
| Hemophilia-B | Abnormal bleeding | Coagulation Factor IX, X- |
| | | chromosomal recessive |
| Hereditary Spherocytosis | spherical-shaped | Ankyrin (ANK1) |
| (various types) | erythrocytes | |
| | (spherocytes) | |
| Paroxysmal nocturnal | Anemia and presence of | PIG-A, X-chromosomal |
| hemoglobinuria | blood in the urine | |
| Porphyria cutanea tarda | Overproduction of heme, | Uroporphyrinogen |
| | iron overload | decarboxylase (UROD), |
| | | autosomal recessive |
| Severe combined immune | Due to impaired DNA | Adenosine deaminase, |
| deficiency (SCID) | synthesis severe | autosomal recessive, IL-2R- |
| | immune deficiency in | γ, JAK3, (IL-7R-α, RAG1/2, |
| | humoral and cellular | Artemis, CD3δ, CD3ε |
| | immunity | |
| Sickle-cell anemia | Abnormal hemoglobin | β-Hemoglobin (HB), |
| | (HbS) | autosomal recessive |
| Thalassemia (α- and β form) | Lack of α- or β | Deletion of HBA1 and/or |
| | hemoglobin resulting in | HBA2, |
| | anemia | |
| Von Willebrand disease | Abnormal bleeding, | Autosomal dominant and |
| (three types known, Type-III is | hemorrhage similar to | recessive forms |
| most severe) | hemophilia A and B | |

Cancer

| | | |
|---|---|---|
| Malignant melanoma | P16 mutation leads to | Cyclie dependant kinase |
| | uncontrolled proliferation | inhibitor 2 (CDKN2) |
| | of fibroblasts | |
| Neurofibromatosis (2 types) | Benign tumors on | NF1, NF2, autosomal |
| | auditory nerves leads to | dominant |
| | deafness | |

Deafness (Ear)

| | | |
|---|---|---|
| Deafness | Hearing loss | Deafness-1A (DFNB1), |
| | | autosomal recessive |
| Pendred syndrome | Hearing loss | Pendrin (PDS), autosomal |
| | | recessive |

Heart

| | | |
|---|---|---|
| Ataxia telangiectasia | DNA damage repair | ATM, |
| | disturbed, | |
| Atherosclerosis | Increase of blood | apoE, |
| | cholesterol | |
| LQT Syndrome (Long QT) | Potassium channel | LQT1 and other genes |
| | defect | |
| Von-Hippel Lindau Syndrome | Abnormal growth of | VHL, autosomal dominant |
| | blood vessels, can lead | |
| | to cancer | |
| William's Beuren Syndrome | Deletion of elastin results | Deletion of elastin and LIM |
| | in vascular defects, | kinase genes |
| | supravalvular aortic | |
| | stenosis | |

Metabolic disorders and glycogen storage diseases

| | | |
|---|---|---|
| Adrenoleukodystrophy | Disturbed fatty acid | ABCD1, X-chromosomal |
| | transport and | |
| | metabolism | |
| Alkaptonuria | Nitrogen metabolism | Homogentisic Oxidase, |
| | defect, Urine turns dark | autosomal recessive |
| | when exposed to oxygen | |
| Diabetes type I | Disturbed insulin | IDDM1, IDDM2, GCK, . . . |
| | production | |
| Galactosemia | disorder of galactose | Galactose-1-phosphate |
| | metabolism | uridyltransferase gene |
| | | (GALT), autosomal |
| | | recessive |
| Gauche disease | Disturbance of fat | Glucocerebrosidase |
| | metabolism | |
| Glucose Galactosidase | Disturbed glucose and | SGLT1, autosomal |
| Malabsorption | galactose transport out | recessive |
| | of the intestinal lumen | |
| | resulting in diarrhea | |
| Glycogen storage disease | Accumulation of glucose | Glucose-6-Phosphatase, |
| Type I, Von-Gierke's disease | in liver and kidney | autosomal recessive |
| Glycogen storage disease | Accumulation of | α-1-Glucosidase, |
| Type II, Pompe's disease | glycogen in liver, heart, | autosomal recessive |
| | skeletal muscle, | |
| | cardiomegaly | |
| Glycogen storage disease | Accumulation of | Debranching enzyme, |
| Type III, Cori's disease | glycogen in liver, heart, | autosomal recessive |
| | skeletal muscle, | |
| | hepatoomegaly | |
| Glycogen storage disease | Cannot untilize glycogen | Muscle phosphorylase, |
| Type V, McArdle's disease | in muscle cells | autosomal recessive |
| Glucose-6-Phosphate | Inability to maintain | G6PD, X-chromosomal |
| Dehydrogenase | glutathione leads to | recessive |
| | hemolytic anemia | |
| Hereditary Hemochromatosis | Excess of iron in the | Hemochromatosis (HFE) |
| (4 types) | body (esp. liver) due to | |
| | excessive iron | |
| | absorption in the gut | |
| Homocystinuria | Nitrogen metabolism | Cystathione synthetase |
| | defect | defect, autosomal recessive |
| Lesh Nyhan Syndrome | Accumulation of uric acid | HPRT1, X-chromosomal |
| | leading to gout, ureate | |
| | stones and muscle loss | |
| Maple Syrup Urine Disease | Amino acid metabolism | Branched-chain-alpha- |
| | defect leads to the | dehydrogenase (BCKDH) |
| | accumulation of α- | |
| | Ketoacides and death in | |
| | the first months if | |
| | untreated | |
| Menkes' Syndrome | Reduced ability to | ATP7A, X-chromosomal |
| | absorb copper, leads to | recessive |
| | death in infancy if | |
| | untreated | |
| Obesity | Elevated body weight | Polygenic, elevated leptin |
| | | levels may play a role |
| Phenylketonuria | Inability to break down | Phenylalanine hydroxylase |
| | Phenylalanine into | (PAH), autosomal recessive |
| | tyrosine leads to mental | |
| | retardation | |
| Tangier disease | reduced levels of plasma | ATP-binding cassette-1 |
| | high density lipoproteins | gene (ABCA1) |
| Zellweger Syndrome (leads to | High levels of iron and | PXR1 (receptor on the |
| death in infants) | copper in the blood | surface of peroxisomes) |
| Wilsons Disease | Copper accumulation in | ATP7B (P-type ATPase), |
| | brain and liver | autosomal recessive |

Musculoskeletal system

| | | |
|---|---|---|
| Achondroplasis | Short stature with a large | Fibroblast growth factor |
| | head due to slow | receptor 3 (FGF3R), |
| | proliferation of | |
| | chondrocytes | |
| Charcot-Marie-Tooth | Degeneration of the | Different forms caused by |
| Syndrome and its more | muscles in limbs | different gene mutations, |
| severe form Dejerine-Sottas | | autosomal recessive and X- |
| Syndrome | | chromosomal |
| Cockayne syndrome (2 types) | Premature aging and | group 8 excision repair |
| | short stature, loss of “on | cross-complementing |
| | the fly” DNA repair | protein (ERCC8) |
| Chondroectodermal dysplasia | Malformation of bones | EVC, autosomal recessive |
| | and polydactyly | |
| Diastrophic dysplasia (DTD) | Malformed hands, | DTDST gene |
| | sulfate transporter defect | |
| Duchenne muscular | Enlargement of muscle | DMD, X-chromosomal |
| dystrophy | tissue with subsequent | recessive |
| | loss of function | |
| Fibrodysplasia Ossificans | Heterotopic bone | NOG, BMP, Autosomal |
| Progressiva | formation | dominant |
| Friedreich's ataxia | Heart enlargement and | Frataxin, autosomal |
| | progressive loss of | recessive |
| | muscular coordination | |
| Hypophosphatasia | Production of an | ALPL, autosomal recessive |
| | abnormal version of | |
| | alkaline phosphatase | |
| | affecting the | |
| | mineralization process | |
| Marfan Syndrome | Connective tissue | Fibrillin 1 (FBN), autosomal |
| | disorder due fibrillin | dominant |
| | deficiency | |
| Myotonic dystrophy (onset | Protein kinase defect in | Dystrophia myotonica |
| during young adulthood) | skeletal muscle cells | protein kinase (DMPK), |
| | | autosomal dominant |
| Osteogenesis imperfect | Defect in type-I collagen | COL1A1, COL1A2 |
| (various types) | formation leads to | |
| | multiple fractures after | |
| | birth | |
| Prader-Willi Syndrome | Decreased muscle tone | SNRPN (small |
| | and mental retardation | ribinucleoprotein N) deleted |
| | | due to a deletion on |
| | | chromosome 15 |

Neurons and Brain

| | | |
|---|---|---|
| Alzheimer disease | Increased amyloid | Polygenic, PS1, PS2, . . . |
| | production, progressive | |
| | inability to remember | |
| | facts | |
| Amyotrophic lateral sclerosis | Progressive | Superoxide dismutase 1 |
| (ALS) (various forms) | degeneration of motor | (SOD1), various genes |
| | neuron cells (defect in | involved |
| | elimination superoxide | |
| | radicals) | |
| Angelman syndrome | Mental retardation with | Genomic imprinting on |
| | inadequate laughing | chromosome 15 |
| Pyruvat dehydrogenase | Neurological defects if | Pyruvat dehydrogenase, |
| | untreated | autosomal recessive |
| Refsum disease | Accumulation of phytanic | Phytanoyl-CoA hydroxylase |
| | acid leads to peripheral | (PHYH), autosomal |
| | neuropathy | recessive |
| Rett's syndrome | Mental retardation with | Methyl-CpG-binding |
| | arrested development | protein-2 (MECP2), X- |
| | between 6 and 18 | chromosomal dominant |
| | months of age | |
| Tay-Sachs disease (various | Disturbed break down of | HEXA (β-hexosaminidas A), |
| forms of severity) | GM2 ganglioside leads | autosomal recessive |
| | to neurological damage | |
| LaFora Disease | Aggressive form of | EPM2A, autosomal |
| | epilepsy | recessive |
| Essential tremor (variable | Uncontrollable shaking | ETM1, ETM2, autosomal |
| forms) | | dominant |
| Fragile X syndrome | Lack of FMR1 RNA | FMR1 gene is not |
| | binding protein, mental | expressed due to an CGG |
| | retardation | amplification in the 5′UTR |
| | | region |
| Huntington's disease | Progressive dementia | HTT (huntingtin), autosomal |
| | with onset in adulthood | dominant |

Intestine

| | | |
|---|---|---|
| Bartter's syndrome (3 types) | Renal disease | Kidney chloride channel B |
| | | gene (CLCNKB), autosomal |
| | | recessive |
| Polycystic kidney disease (2 | renal disease | PDK1, PDK2, autosomal |
| types) | | dominant, there is also a |
| | | autosomal recessive form |
| | | known (ARPKD) |

Lung

| | | |
|---|---|---|
| Alpha-1-antitrypsin | Defect alveoli due to | SERPINA1, autosomal |
| | uncontrolled release of | codominant |
| | elastase | |
| Asthma | Chronic inflammatory | Polygenic |
| | disorder of the airways | |
| Cystic fibrosis | Excessively viscous | CFTR (cystic fibrosis |
| | mucous due to defective | conductance |
| | Cl⁻ ion transport | transmembrane regulator), |
| | | autosomal recessive |
| Surfactant metabolism | Newborns are of normal | ATP-binding cassette |
| dysfunction (various types) | body weight, but all fail to | transporter (ABCA3) |
| | inflate | |
| Primary cliliary dyskinesia | Excessively viscous | DNAI1, CCNO, CCDC40 |
| | mucous due to | among others |
| | defective/missing cilia | |
| | function | |

Lysosomal storage diseases

| | | |
|---|---|---|
| Fabry's disease | Beyond others, skin | α-Galactosidase A, X- |
| | lesions due to the | chromosomal recessive |
| | accumulation of | |
| | ceramide trihexoside | |
| Gaucher's Disease | Accumulation of | Glucocerebrosidase, |
| Type-I: adult form (normal | glucocerebrosides | autosomal recessive, |
| lifespan under treatment) | (gangliosides, | |
| Type-II: infantile form (death | sphingolipids) | |
| before age 1) | | |
| Type-III: juvenile form (onset | | |
| in early childhood, less severe | | |
| than Type-II) | | |
| Hunter's Syndrome | Accumulation of | L-iduronosulfat sulfatase, X- |
| | mucopolysaccharides | chromosomal recessive |
| Hurler's Syndrome (death by | Accumulation of | α-L-iduronidase, autosomal |
| age of 10) | mucopolysaccharides | recessive |
| Niemann-Pick Disease (three | Defect in releasing | Sphingomyelinase, |
| distinct forms A, B, C) | Cholesterol from | autosomal recessive |
| | lysosomes, | |
| | accumulation of | |
| | Sphingomyelin | |
| Tay-Sachs disease (death by | Accumulation of G_{M2} | Hexosaminidase A, |
| age of 4) | ganglioside in neuronal | autosomal recessive |
| | cells | |

Skin

| | | |
|---|---|---|
| Albinism | Nitrogen metabolism | Tyrosinase deficiency, |
| | defect | autosomal recessive |
| Albinism, oculocutaneous, | Reduced biosynthesis | OCA2, autosomal recessive |
| type II | of melanin pigment | |
| Ehlers-Danlos Syndrome | Diaphragmatic hernia. | Various defects in collagen |
| (various types) | common, retinal | synthesis |
| | detachment | |
| Epidermolysis bullosa | Defects in maintenance | Epidermolysis bullosa |
| (various types including EB | of keratinocyte | macular type (EBM), |
| simplex, Junctional EB, | structural stability or | Epidermolysis bullosa 3 |
| Dystrophic EB and Kindler | adhesion of the | progressiva (EBR3), |
| syndrome) | keratinocyte to the | Epidermolysis bullosa 4 |
| | underlying dermis | pseudojunctual (EBR4), |
| | | Desmoplakin (DSP), |
| | | Plakophilin-1 (PKP1), |
| | | kreatin (KRT5, KRT14), |
| | | plectin (PLEC), ITGA6, |
| | | integrin subunit (ITGB4), |
| | | laminin subunits (LAMA3, |
| | | LAMP3, LAMB3, LAMC2), |
| | | collagen (COL17A1, |
| | | COL7A1 (autosomal |
| | | dominant), FERMT1, |
| | | autosomal recessive |
| Hartnup's disease | Defect in tryptophan | SLC6A19, autosomal |
| | uptake in the | recessive |
| | gastrointestinal tract, | |
| | light-sensitive skin | |
| Hereditary Hemorrhagic | Telangiectasia of the | Endoglin (ENG), autosomal |
| Telangiectasia, Osler-Weber- | skin and mucous | dominant |
| Rendu Syndrome | membranes | |
| Hypercholesterolemia, familial | elevation of serum | Low-density lipoprotein |
| | cholesterol bound to low | receptor (LDLR), |
| | density lipoprotein, | apolipoprotein B (APOB), |
| | accumulation in skin | autosomal dominant |
| | and arteriosclerosis | |
| Xeroderma pigmentosa | skin defect and | DNA repair defect, |
| | melanoma due to UV | autosomal recessive |
| | exposure | |
| Male pattern baldness | Disturbed conversion of | 5-α-reductase |
| | testosterone into | |
| | dihydrotestosterone in | |
| | the skin | |

Genetic liver diseases

| | | |
|---|---|---|
| Amino acid metabolism | Disruptions in the | FAH, TAT, HPD, autosomal |
| disorders | multistep process that | recessive |
| | breaks down the amino | |
| | acid tyrosine and | |
| | phenylalanine | |
| Beta-thalassemia intermedia | Shortage of mature red | HBB, autosomal recessive |
| | blood cells | |
| Crigler-Najjar syndrome | Deficiency in | UGT1A1, autosomal |
| | glucuronidation in which | recessive |
| | bilirubin gets | |
| | dissolvable in water | |
| Fatty acid oxidation disorders | Deficiency in processing | HADHA, ACADVL |
| | of long-chain fatty acids | autosomal recessive |
| | and very long-chain | |
| | fatty acids resulting in | |
| | lethargy and | |
| | hypoglycemia | |
| Fructose metabolism | Impaired | FBP1, ALDOB, autosomal |
| disorders | gluconeogenesis | recessive |
| | causing hypoglycemia | |
| Galactosemia | Deficiency in processing | GALT, GALK1, GALE, |
| | galactose | autosomal recessive |
| Glycogen storage diseases | Disturbed breackdown | G6PC, SLC37A4, AGL, |
| | of glucose 6-phosphate | GBE1, autosomal |
| | and glycogen leads to | recessive |
| | accumulation of | |
| | glycogen as well as | |
| | abnormal glycogen | |
| | molecules causing cell | |
| | damage | |
| Heme biosynthesis disorder | Decrease of | UROD autosomal |
| | uroporphyrinogen | dominant, ALAS2 X-limked |
| | decarboxylase resulting | dominant, ALAD |
| | in accumulation of | autosomal recessive |
| | compounds called | |
| | porphyrins causing | |
| | toxic levels in liver | |
| Lipid metabolism (transport) | Shortage of functional | NPC1, NPC2 autosomal |
| disorders | protein, which prevents | recessive, LDLR, |
| | movement of | autosomal dominant |
| | cholesterol and other | |
| | lipids, leading to their | |
| | accumulation in cells | |
| Metal metabolism disorders | Disorders in the storage | ATP7B, HAMP, HFE, |
| | and transport of iron and | HFE2, autosomal recessive |
| | copper resulting in | |
| | accumulation in tissues | |
| | and organs | |
| Organic acid disorders | Disrupted break down of | BCKDHA, BCKDHB, and |
| (Acidurias/Acidemias) | several protein building | DBT, PCCA and PCCB, |
| | blocks (amino acids), | MUT, MMAA, MMAB, |
| | certain lipids, and | MMADHC, MCEE, IVD, |
| | cholesterol | MCCC1 or MCCC2, |
| | | autosomal recessive |
| Primary hyperoxaluria type 1 | Disrupted breakdown of | AGXT, GRHPR, autosomal |
| | glyoxylate leading to | recessive |
| | renal damage | |
| Progressive familial | Buildup of bile acids in | ATP8B1, autosomal |
| intrahepatic cholestasis | liver cells causing liver | recessive |
| | damage | |
| Thrombocyte activity disorder | Lack of enzyme activity | ADAMTS13, autosomal |
| | disrupts the usual | recessive |
| | balance between | |
| | bleeding and clotting | |
| Urea cycle disorders | Disorder of the urea | OTC (X-linked disorder), |
| | cycle which causes a | CPS1, ASS1 and |
| | form of | SLC25A13, ASL, autosomal |
| | hyperammonemia | recessive |

The above Table A shows examples of genes in which a defect leads to a disease which can be treated with the RNA, preferably the mRNA, which may be present in the suspension formulation and the aerosol of the present invention wherein RNA, preferably the mRNA, comprises a ribonucleotide sequence which encodes an intact version of the protein or a functional fragment thereof of the above disclosed defective gene. In particularly preferred embodiments, hereditary diseases can be addressed, which for example affect the lungs, such as SPB (surfactant protein B) deficiency, ABCA3 deficiency, cystic fibrosis and al-antitrypsin deficiency, or which affect plasma proteins (e.g. congenital hemochromatosis (hepcidin deficiency), thrompotic thrombocytopenic purpura (TPP, ADAMTS 13 deficiency) and cause clotting defects (e.g. haemophilia a and b) and complement defects (e.g. protein C deficiency), immune defects such as for example SCID (caused my mutations in different genes such as: RAG1, RAG2, JAK3, IL7R, CD45, CD3δ, CD3ε) or by deficiencies due to lack of adenosine desaminase for example (ADA-SCID), septic granulomatosis (e.g. caused by mutations of the gp-91-phox gene, the p47-phox gene, the p67-phox gene or the p33-phox gene) and storage diseases like Gaucher's disease, Fabry's disease, Krabbe's disease, MPS I, MPS II (Hunter syndrome), MPS VI, Glycogen storage disease type II or mucopolysaccharidoses.

Other disorders for which the RNA, preferably the mRNA, of the present invention can be useful include disorders such as SMN1-related spinal muscular atrophy (SMA); amyotrophic lateral sclerosis (ALS); GALT-related galactosemia; Cystic Fibrosis (CF); SLC3A1-related disorders including cystinuria; COL4A5-related disorders including Alport syndrome; galactocerebrosidase deficiencies; X-linked adrenoleukodystrophy and adrenomyeloneuropathy; Friedreich's ataxia; Pelizaeus-Merzbacher disease; TSC1 and TSC2-related tuberous sclerosis; Sanfilippo B syndrome (MPS IIIB); CTNS-related cystinosis; the FMR₁-related disorders which include Fragile X syndrome, Fragile X-Associated Tremor/Ataxia Syndrome and Fragile X Premature Ovarian Failure Syndrome; Prader-Willi syndrome; hereditary hemorrhagic telangiectasia (AT); Niemann-Pick disease Type C1; the neuronal ceroid lipofuscinoses-related diseases including Juvenile Neuronal Ceroid Lipofuscinosis (JNCL), Juvenile Batten disease, Santavuori-Haltia disease, Jansky-Bielschowsky disease, and PTT-1 and TPP1 deficiencies; EIF2B1, EIF2B2, EIF2B3, EIF2B4 and EIF2B5-related childhood ataxia with central nervous system hypomyelination/vanishing white matter; CACNA1A and CACNB4-related Episodic Ataxia Type 2; the MECP2-related disorders including Classic Rett Syndrome, MECP2-related Severe Neonatal Encephalopathy and PPM-X Syndrome; CDKLS-related Atypical Rett Syndrome; Kennedy's disease (SBMA); Notch-3 related cerebral autosomal dominant arteriopathy with subcortical infarcts and leukoencephalopathy (CADASIL); SCN1A and SCN1B-related seizure disorders; the Polymerase G-related disorders which include Alpers-Huttenlocher syndrome, POLG-related sensory ataxic neuropathy, dysarthria, and ophthalmoparesis, and autosomal dominant and recessive progressive external ophthalmoplegia with mitochondrial DNA deletions; X-Linked adrenal hypoplasia; X-linked agammaglobulinemia; Fabry disease; and Wilson's disease.

In all these diseases, a protein, e.g. an enzyme, is defective, which can be treated with the RNA, preferably the mRNA, encoding any of the above proteins of the present invention, which makes the protein encoded by the defective gene or a functional fragment thereof available. Transcript replacement therapies/protein replacement therapies do not affect the underlying genetic defect, but increase the concentration of the protein in which the subject is deficient. As an example, in Pompe's disease, the transcript replacement therapy/enzyme replacement therapy replaces the deficient lysosomal enzyme acid alpha-glucosidase (GAA).

Thus, non-limiting examples of proteins which can be encoded by the mRNA of the present invention are erythropoietin (EPO), growth hormone (somatotropin, hGH), cystic fibrosis transmembrane conductance regulator (CFTR), growth factors such as GM-SCF, G-CSF, MPS, protein C, hepcidin, ABCA3 and surfactant protein B. Further examples of diseases which can be treated with the RNA according to the invention are hemophilia A/B, Fabry's disease, CGD, ADAMTS13, Hurler's disease, X chromosome-mediated A-γ-globulinemia, adenosine deaminase-related immunodeficiency and respiratory distress syndrome in the newborn, which is linked with SP-B. Particularly preferably, the RNA, preferably the mRNA, according to the invention contains the coding sequence for surfactant protein B (SP-B) or for erythropoietin. Further examples of proteins which can be encoded by the RNA, preferably the mRNA, of the present invention according to the invention are growth factors such as human growth hormone hGH, BMP-2 or angiogenesis factors.

Although the above embodiments are described in the context of the RNA, preferably an mRNA molecule, that may be present in the nanoparticles used in the present invention, the present invention, as mentioned above, is not limited to the use of an RNA, preferably an mRNA but may employ other nucleic acid molecules, such as DNA molecules.

Said DNA molecule may encode the above RNA, preferably the above mRNA and, accordingly, harbour the genetic information for the correspondingly transcribed RNA molecule.

Hence, as regards preferred embodiments the same applies, mutatis mutandis, to the DNA molecule of the present invention as has been set forth above and below in the context of the RNA molecule, preferably the mRNA molecule, that may be present in the nanoparticles used in the present invention.

Alternatively, the RNA, preferably the mRNA, may contain a ribonucleotide sequence which encodes a full-length antibody or a smaller antibody (e.g., both heavy and light chains) which can be used in therapeutic settings to, e.g., confer immunity to a subject. Corresponding antibodies and their therapeutic application(s) are known in the art. The antibody may be encoded by a single mRNA strand or by more than one mRNA strand.

In another embodiment, the RNA, preferably the mRNA may encode a functional monoclonal or polyclonal antibody, which may be useful for targeting and/or inactivating a biological target (e.g., a stimulatory cytokine such as tumor necrosis factor). Similarly, the RNA, preferably the mRNA sequence may encode, for example, functional anti-nephrotic factor antibodies useful for the treatment of membranoproliferative glomerulonephritis type II or acute hemolytic uremic syndrome, or alternatively may encode anti-vascular endothelial growth factor (VEGF) antibodies useful for the treatment of VEGF-mediated diseases, such as cancer.

In another embodiment, the RNA, preferably the mRNA may encode a functional monoclonal or polyclonal antibody, which may be useful for neutralizing or otherwise inhibiting a virus or virus replication.

Alternatively, the RNA, preferably the mRNA, may contain a ribonucleotide sequence which encodes an antigen which preferably can be used in preventive or therapeutic settings.

In another embodiment, the mRNA may encode a protein or proteins that can induce an immune modulation, such as cytokines, including chemokines, interferons (such as interferon lambda), interleukins, lymphokines, and tumour necrosis factors.

In another embodiment, the RNA, preferably the mRNA, may contain a ribonucleotide sequence which encodes a polypeptide or a protein which can be used in genome editing technologies. Genome editing is a type of genetic engineering in which DNA is inserted, deleted or replaced in the genome of an organism using nucleases. These nucleases create site-specific breaks at desired locations in the genome. The induced breaks are repaired by non-homologous end-joining or homologous recombination, resulting in targeted mutations in the genome, thereby “editing” the genome. The breaks may either be single-strand breaks or double-strand breaks (DSBs) while double-strand breaks (DSBs) are preferred. Numerous genome editing systems utilizing different polypeptides or proteins are known in the art, i.e., e.g., the CRISPR-Cas system, meganucleases, zinc finger nucleases (ZFNs) and transcription activator-like effector-based nucleases (TALEN). Methods for genome engineering are reviewed in Trends in Biotechnology, 2013, 31 (7), 397-405.

Thus, in a preferred embodiment, the RNA, preferably the mRNA, may contain a ribonucleotide sequence which encodes a polypeptide or protein of the Cas (CRISPR associated protein) protein family, preferably Cas9 (CRISPR associated protein 9). Proteins of the Cas protein family, preferably Cas9, may be used in CRISPR/Cas9 based methods and/or CRISPR/Cas9 genome editing technologies. CRISPR-Cas systems for genome editing, regulation and targeting are reviewed in Nat. Biotechnol., 2014, 32(4):347-355.

In another preferred embodiment, the RNA, preferably the mRNA, may contain a ribonucleotide sequence which encodes a meganuclease. Meganucleases are endodeoxyribonucleases which, in contrast to “conventional” endodeoxyribonucleases, recognize a large recognition site (e.g., a double-stranded DNA sequence of 12 to 40 base pairs). As a result, the respective site occurs only a few times, preferably only once, in any given genome. Meganucleases are therefore considered to be the most specific naturally occurring restriction enzymes and, accordingly, are suitable tools in genome editing technologies.

In another preferred embodiment, the RNA, preferably the mRNA, contains a ribonucleotide sequence which encodes a zinc finger nuclease (ZFN). ZFNs are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of the endogenous DNA repair machinery, ZFNs can be used to precisely alter the genome of higher organisms and are, therefore, suitable tools in genome editing technologies.

In another preferred embodiment, the RNA, preferably the mRNA, may contain a ribonucleotide sequence which encodes a transcription activator-like effector nuclease (TALEN). TALENs are restriction enzymes that can be engineered to cut specific sequences of DNA. TALENs are fusion proteins wherein a TAL effector DNA-binding domain is fused to a DNA cleavage domain of a nuclease. Transcription activator-like effectors (TALEs) can be engineered to bind practically any desired DNA sequence. Thus, when combined with a nuclease, DNA can be cut at specific desired locations.

Although the above embodiments are described in the context of the RNA, preferably an mRNA molecule, the present invention, as mentioned above, is not only limited to the use of an RNA, preferably an mRNA, but may employ any nucleic acid molecule, such as a DNA molecule.

Said DNA molecule may encode the above RNA, preferably the above mRNA and, accordingly, harbour the genetic information for the correspondingly transcribed RNA molecule.

Hence, as regards preferred embodiments the same applies, mutatis mutandis, to the DNA molecule as has been set forth above and below in the context of the RNA molecule, preferably the mRNA molecule, that may be present in the nanoparticles used in the present invention.

Alternatively to the above, the RNA contains a ribonucleotide sequence which is not to be expressed as a protein or a polypeptide. Thus, the term RNA should not only be understood to mean any polynucleotide molecule which, if introduced into a cell, is translatable to a polypeptide/protein or fragment thereof. Rather, it is also contemplated that the RNA contains a ribonucleotide sequence which is not translated into a protein. In this context, it is envisaged that the RNA contains a ribonucleotide sequence which preferably provides the genetic information for an antisense RNA, an siRNA or a miRNA sequence or another desired non-coding ribonucleotide sequence.

Thus, the RNA may also be an antisense RNA, an siRNA or a miRNA sequence. Antisense RNA, siRNA or miRNA sequences can be used to silence the effect of a certain RNA molecule at some stage. This may, in particular, be desirable and useful in certain medical settings and in the treatment of a certain disease and, in particular, in RNA-based therapies as described herein above and below.

Silencing the effect of an RNA molecule can be achieved by making use of an RNAi (RNA interference) mechanism by using the nucleic acid strand which is complementary to a certain RNA sequence. The term “RNA interference” or “inhibiting RNA” (RNAi/iRNA) describes the use of double-stranded RNA to target specific mRNAs for degradation, thereby silencing their translation. Preferred inhibiting RNA molecules may be selected from the group consisting of double-stranded RNA (dsRNA), siRNA, shRNA and stRNA. dsRNA matching a gene sequence may be synthesized in vitro and introduced into a cell. The dsRNA may also be introduced into a cell in form of a vector expressing a target gene sequence in sense and antisense orientation, for example in form of a hairpin mRNA. The sense and antisense sequences may also be expressed from separate vectors, whereby the individual antisense and sense molecules form double-stranded RNA upon their expression. It is known in the art that in some occasions the expression of a sequence in sense orientation or even of a promoter sequence suffices to give rise to dsRNA and subsequently to siRNA due to internal amplification mechanisms in a cell. Accordingly, all means and methods which result in a decrease in activity of the polypeptide or protein encoded by the coding region are to be used in accordance with the present invention. For example sense constructs, antisense constructs, hairpin constructs, sense and antisense molecules and combinations thereof can be used to generate/introduce these siRNAs. The dsRNA feeds into a natural process including the highly conserved nuclease dicer which cleaves dsRNA precursor molecules into short interfering RNAs (siRNAs). The generation and preparation of siRNA(s) as well as the method for inhibiting the expression of a target gene is, inter alia, described in WO 02/055693, Wei (2000) Dev. Biol. 15:239-255; La Count (2000) Biochem. Paras. 111:67-76; Baker (2000) Curr. Biol. 10:1071-1074; Svoboda (2000) Development 127:4147-4156 or Marie (2000) Curr. Biol. 10:289-292. These siRNAs build then the sequence specific part of an RNA-induced silencing complex (RISC), a multicomplex nuclease that destroys messenger RNAs homologous to the silencing trigger). Elbashir (2001) EMBO J. 20:6877-6888 showed that duplexes of 21 nucleotide RNAs may be used in cell culture to interfere with gene expression in mammalian cells.

Methods to deduce and construct siRNAs are known in the art and are described in Elbashir (2002) Methods 26:199-213, at the internet web sites of commercial vendors of siRNA, e.g. Qiagen GmbH (https://www1.qiagen.com/GeneGlobe/Default.aspx); Dharmacon (www.dharmacon.com); Xeragon Inc. (http://www.dharmacon.com/Default.aspx), and Ambion (www.ambion.com), or at the web site of the research group of Tom Tuschl (http://www.rockefeller.edu/labheads/tuschl/sirna.html). In addition, programs are available online to deduce siRNAs from a given mRNA sequence (e.g. http://www.ambion.com/techlib/misc/siRNA_finder.html or http://katandin.cshl.org:9331/RNAi/html/rnai.html). Uridine residues in the 2-nt 3′ overhang can be replaced by 2′deoxythymidine without loss of activity, which significantly reduces costs of RNA synthesis and may also enhance resistance of siRNA duplexes when applied to mammalian cells (Elbashir (2001) loc. cit). The siRNAs may also be sythesized enzymatically using T7 or other RNA polymerases (Donze (2002) Nucleic Acids Res 30:e46). Short RNA duplexes that mediate effective RNA interference (esiRNA) may also be produced by hydrolysis with Escherichia coli RNase III (Yang (2002) PNAS 99:9942-9947). Furthermore, expression vectors have been developed to express double stranded siRNAs connected by small hairpin RNA loops in eukaryotic cells (e.g. (Brummelkamp (2002) Science 296:550-553). All of these constructs may be developed with the help of the programs named above. In addition, commercially available sequence prediction tools incorporated in sequence analysis programs or sold separately, e.g. the siRNA Design Tool offered by www.oligoEngine.com (Seattle,WA) may be used for siRNA sequence prediction.

microRNA (miRNA) resemble small interfering RNAs (siRNAs) described above. microRNA (miRNA) is a small non-coding RNA molecule (containing about 22 nucleotides) found in plants, animals and some viruses, that functions in RNA silencing and post-transcriptional regulation of gene expression. miRNAs function via base-pairing with complementary sequences within mRNA molecules. As a result, these mRNA molecules are silenced, by one or more of the following processes: (1) cleavage of the mRNA strand into two pieces, (2) destabilization of the mRNA through shortening of its poly(A) tail, and (3) less efficient translation of the mRNA into proteins by ribosomes. As mentioned, miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA.

A DNA molecule used in the suspension formulations and the aerosol of the present invention may also be one which encodes the above RNA, e.g. the above siRNA or miRNA, accordingly, harbours the genetic information for the correspondingly transcribed RNA molecule. Hence, as regards preferred embodiments the same applies, mutatis mutandis, to the DNA molecule as has been set forth above in the context of the RNA molecule, preferably the mRNA molecule, that may be present in the nanoparticles used in the present invention.

It will be understood that the nanoparticles in the context of the present invention can comprise a single type of nucleic acid, preferably an RNA such as mRNA, but may alternatively comprise a combination of two or more types of nucleic acids, preferably RNAs, e.g. in the form of particles comprising two or more types of nucleic acids, preferably RNAs, in single particles, or in the form of a blend of particles which differ in the type of nucleic acid, preferably RNA such as mRNA, contained therein.

As explained above, the nanoparticles of the aqueous suspension formulation in accordance with the invention and of the aerosol in accordance with the invention further comprise, as component (b), an ionizable lipid or an ionizable lipidoid. It will be understood that this encompasses the possibility that the nanoparticles comprise a combination of different ionizable lipids, a combination of different ionizable lipidoids, or a combination of one or more ionizable lipids and one or more ionizable lipidoids. The nanoparticles used in the context of the present invention typically comprise the nucleic acid (a) and the ionizable lipid or the ionizable lipidoid (b) in the form of a mixture of these components.

The terms “ionizable lipid” and “ionizable lipidoid”, are used in the field of lipid nanoparticles and lipidoid nanoparticles to refer to a lipid or a lipidoid which is protonated to carry a cationic charge, or which can be protonated to carry a cationic charge. Thus, ionizable lipids and lipidoids, respectively, are also referred to as “protonatable lipids” and “protonatable lipidoids”, or as titratable lipids or titratable lipidoids, respectively. As will be understood by the skilled reader, the reference to an “ionizable lipid” or an “ionizable lipidoid” encompasses the ionizable lipid or lipidoid in its protonated or non-protonated form. As will further be understood, the protonated or non-protonated state of the lipid or lipidoid is generally determined by the pH value of a medium surrounding the lipid or lipidoid, e.g. by the pH value of the aqueous vehicle solution comprised in the aqueous suspension formulation and by the aerosol in accordance with the invention.

Counterions (anions) for the positive charges of positively charged ionizable lipids or ionizable lipidoids in the context of the invention are typically provided by anionic moieties contained in the nucleic acid. If positively charged groups are present in excess compared to the anionic moieties in the nucleic acid, positive charges may be balanced by other pharmaceutically acceptable anions, such as chloride, bromide, or iodide, sulfate, nitrate, phosphate, hydrogenphosphate, dihydrogenphosphate, carbonate, or hydrogencarbonate, or by a polyanion component different from the nucleic acid, which may be present as an optional component in the nanoparticles.

Ionizable lipids and ionizable lipidoids are well known as components of lipid nanoparticles or lipidoid nanoparticles. In the context of the present invention, there are no particular restrictions imposed on the type of ionizable lipid or ionizable lipidoid contained in the nanoparticles.

Generally, an ionizable lipid or lipidoid, respectively, comprises a primary, secondary or tertiary amino group which can act as proton acceptor and which may thus be protonated or non-protonated. An ionizable lipidoid generally comprises a plurality of such amino groups, such as two or more, preferably three or more.

Preferably, an ionizable lipid which may be comprised by the nanoparticles used in the suspension formulation and in the aerosol in accordance with the invention is a lipid which comprises a protonatable head group which contains one or more, preferably one, primary, secondary or tertiary amino group(s) as a protonatable or protonated group, and one or more, preferably one or two, hydrophobic moieties, linked to the head group.

Examples of these preferred ionizable lipids are
i) a lipid which comprises a protonatable head group which contains one or more, preferably one, primary, secondary or tertiary amino group(s) as a protonatable or protonated group, and one hydrophobic moiety linked to the head group;
ii) a lipid which comprises one secondary or tertiary amino group as a protonatable or protonated head group, and two hydrophobic moieties linked to the head group.

A hydrophobic moiety comprised in these preferred lipids preferably contains one or more of a linear chain aliphatic residue, e.g. a linear chain residue comprising 8 to 18 carbon atoms, a branched chain aliphatic residue, e.g. a branched chain residue comprising 8 to 18 carbon atoms, or an alicyclic ring structure which may be a condensed ring structure, e.g. an alicyclic ring structure comprising 10 to 18 carbon atoms. In addition, the hydrophobic moiety may comprise one or more linking groups which facilitate the linking of the moiety to the head group, or which allow two or more of the above aliphatic residues to be combined with each other. Furthermore, it may comprise one or more substituents, to the extent that the hydrophobic characteristics of the moiety are maintained.

Preferably, an ionizable lipidoid which may be comprised in the nanoparticles used in the suspension formulation and in the aerosol in accordance with the invention is an oligoamine, more preferably an oligoalkylamine, which comprises at least two, preferably at least three, amino groups selected from a protonatable or protonated secondary and a tertiary amino group, each of which may carry a hydrophobic moiety attached to it. In addition to the amino groups carrying a hydrophobic residue, the lipidoid may comprise further protonatable or protonated amino groups selected from a primary, a secondary and a tertiary amino group. Preferably, the total number of the amino groups is 3 to 10, more preferably 3 to 6. Preferably, the total number of hydrophobic moieties attached to the amino groups is 3 to 6. Preferably, the ratio of the number of hydrophobic moieties attached to amino groups to the total number of amino groups in the oligoalkylamine is 0.75 to 1.5

A hydrophobic moiety comprised in such a preferred lipidoid preferably contains one or more of a linear chain aliphatic residue, e.g. a linear chain residue comprising 8 to 18 carbon atoms and a branched chain aliphatic residue, e.g. a branched chain residue comprising 8 to 18 carbon atoms. In addition, the hydrophobic moiety may comprise one or more linking groups which facilitate the linking of the moiety to an amino group, or which allow two or more of the above aliphatic residues to be combined with each other. Furthermore, it may comprise one or more substituents, to the extent that the hydrophobic characteristics of the moiety are maintained.

Suitable exemplary ionizable lipids or ionizable lipidoids which can be comprised as component (bl) in the in the nanoparticles used in the context of the present invention are disclosed, e.g., in WO 2006/138380 A2, EP2476756 A1, US 2016/0114042 A1, U.S. Pat. Nos. 8,058,069 B2, 8,492,359 B2, 8,822,668 B2, 8,969,535, 9,006,417 B2, 9,018,187 B2, 9,345,780 B2, 9,352,042 B2, 9,364,435 B2, 9,394,234 B2, 9,492,386 B2, 9,504,651 B2, 9,518,272 B2, DE 19834683 A1, WO 2010/053572 A2, U.S. Pat. No. 9,227,917 B2, 9,556,110 B2, 8,969,353 B2, 10,189,802 B2, WO 2012/000104 A1, WO 2010/053572, WO 2014/028487 or WO 2015/095351, or by Akinc, A., et al., Nature Biotechnology, 26(5), 2008, 561-569; Sabnis, S. et al., Molecular Therapy, 26(6), 2018, Vol. 26 No 6 Jun. 2018, 1509-1519; Kowalski, P. S., et al., Molecular Therapy, 27(4), 2019, 710-728; Kulkarni, J. A. et al, Nucleic Acid Therapeutics, 28(3), 2018, 146-157; and Li, B. et al., Nano Letters, 15, 2015, 8099-8107.

Preferably, component (b) of the nanoparticles comprises or more preferably consists of an ionizable lipidoid of the following formula (la) or a protonated form thereof. The ionizable lipidoid of the following formula (Ia) or its protonated forms which can be used as a preferred component (b) in the context of the present invention are described in detail in the PCT application WO 2014/207231 A1.

Thus, component (b) preferably comprises or consists of a lipidoid of the following formula (b-1)

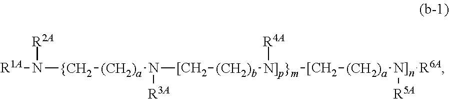

wherein the variables a, b, p, m, n and R_{1A} to R_{6A} are defined as follows:
a is 1 and b is an integer of 2 to 4; or a is an integer of 2 to 4 and b is 1,
p is 1 or 2,
m is 1 or 2; n is 0 or 1 and m+n is≥2; and
R_{1A} to R_{6A} are independently of each other selected from hydrogen; —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A}; —CH₂—R_{7A}; —C(NH)—NH₂; a poly(ethylene glycol) chain; and a receptor ligand; wherein R_{7A} is selected from C3-C18 alkyl and C3-C18 alkenyl having one C—C double bond;
provided that at least two residues among R_{1A} to R_{6A} are selected from —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A} and —CH₂—R_{7A} wherein R_{7A} is selected from C3-C18 alkyl or C3-C18 alkenyl having one C—C double bond;
or a protonated form thereof, wherein one or more of the nitrogen atoms contained in the compound of formula (I) are protonated to provide a compound carrying a positive charge.

Preferably, R_{1A} to R_{6A} are independently selected from hydrogen; a group —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A}; and —CH₂—R_{7A} wherein R_{7A} is selected from C3-C18 alkyl and C3-C18 alkenyl having one C—C double bond; provided that at least two residues among R_{1A} to R_{6A}, more preferably at least three residues among R_{1A} to R_{6A}, and still more preferably at least four residues among R_{1A} to R_{6A} are a group selected from —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A} and —CH₂—R_{7A} wherein R_{7A} is selected from C3-C18 alkyl and C3-C18 alkenyl having one C—C double bond. More preferably, R_{1A} to R_{6A} are independently selected from hydrogen and a group —CH₂—CH(OH)—R_{7A} wherein R_{7A} is selected from C3-C18 alkyl and C3-C18 alkenyl having one C—C double bond; provided that at least two residues among R_{1A} to R_{6A}, more preferably at least three residues among R_{1A} to R_{6A}, and still more preferably at least four residues among R_{1A} to R_{6A} are a group —CH₂—CH(OH)—R_{7A}, wherein R_{7A} is selected from C3-C18 alkyl and C3-C18 alkenyl having one C—C double bond.

Preferably, R_{7A} is selected from C8-C18 alkyl and C8-C18 alkenyl having one C—C double bond, and more preferably from C8-C12 alkyl and C8-C12 alkenyl having one C—C double bond. Generally, alkyl groups are preferred over alkenyl groups as R_{7A}.

As far as any of the groups R_{1A} to R_{6A} is a protecting group for an amino group, such as described for example in WO2006/138380, preferred embodiments thereof are t-butoxycarbonyl (Boc), 9-fluorenylmethoxycarbonyl (Fmoc), or carbobenzyloxy (Cbz).

As far as any of the groups R_{1A} to R_{6A} are a receptor ligand, useful examples are given in Philipp and Wagner in “Gene and Cell Therapy—Therapeutic Mechanisms and Strategy”, 3^{rd} Edition, Chapter 15. CRC Press, Taylor & Francis Group LLC, Boca Raton 2009. Preferred receptor ligands for lung tissue are described in Pfeifer et al. 2010, Ther Deliv. 1(1):133-48. Preferred receptor ligands include synthetic cyclic or linear peptides such as derived from screening peptide libraries for binding to a particular cell surface structure or particular cell type, cyclic or linear RGD peptides, synthetic or natural carbohydrates such as sialic acid, galactose or mannose or synthetic ligands derived from reacting a carbohydrate for example with a peptide, antibodies specifically recognizing cell surface structures, folic acid, epidermal growth factor and peptides derived thereof, transferrin, anti-transferrin receptor antibodies, nanobodies and antibody fragments, or approved drugs that bind to known cell surface molecules.

As far as any of the groups R_{1A} to R_{6A} are a poly(ethylene glycol) chain, the preferred molecular weight of the poly(ethylene glycol) chain is 100-20,000 g/mol, more preferably 1,000-10,000 g/mol and most preferred is 1,000-5,000 g/mol.

The variable pin formula (b-1) is preferably 1.

In formula (b-1), m is 1 or 2; n is 0 or 1 and m+n is≥2. In other words, if m is 1, n must also be 1, and if m is 2, n can be 0 or 1. If n is 0, m must be 2. If n is 1, m can be 1 or 2.

The variable n in formula (b-1) is preferably 1. It is more preferred that m is 1 and n is 1.

Thus, the combination of p=1, m=1 and n=1 is likewise preferred.

As for the variables a and b in formula (la), it is preferred that one of a and b is 1, and the other one is 2 or 3. It is more preferred that a is 1 and b is 2, or that a is 2 and b is 1. Most preferably, a is 1 and b is 2.

In view of the above, it is further preferred that the compound of formula (b-1) is a compound of formula (b-1a) and that component (b) comprises or consists of a lipidoid of the following formula (b-1a):

R_{1A}—NR_{2A}—CH₂—(CH₂)ₐ—NR_{3A}—CH₂—(CH₂)_{b}—NR_{4A}—CH₂—(CH₂)ₐ—NR_{5A}—R_{6A} (b-1a),
wherein a, b, and R_{1A} to R_{6A} are defined as in formula (b-1), including preferred embodiments thereof;
   or a protonated form thereof wherein one or more of the nitrogen atoms indicated in formula (b-1a) are protonated to provide a compound carrying a positive charge.

In accordance with a still further preferred embodiment, the compound of formula (b-1) is a compound of formula (b-1b) and component (b) comprises or consists of a lipidoid compound of the following formula (b-1b),

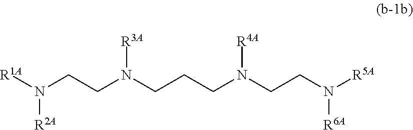

wherein R_{1A} to R_{6A} are defined as in formula (la), including preferred embodiments thereof;
   or a protonated form thereof wherein one or more of the nitrogen atoms indicated in formula (b-1b) are protonated to provide a compound carrying a positive charge.

Thus, in a accordance with a particularly preferred embodiment, component (b) comprises or consists of a lipidoid compound of the above formula (b-1b) or a protonated form thereof, and R_{1A} to R_{6A} are independently selected from hydrogen and —CH₂—CH(OH)—R_{7A}, wherein R_{7A} is selected from C8-C18 alkyl and C8-C18 alkenyl having one C—C double bond, provided that at least two residues among R_{1A} to R_{6A} are —CH₂—CH(OH)—R_{7A}, more preferably at least three residues among R_{1A} to R_{6A}, and still more preferably at least four residues among R_{1A} to R_{6A} are —CH₂—CH(OH)—R_{7A}, wherein R_{7A} is selected from C8-C18 alkyl and C8-C18 alkenyl having one C—C double bond.

In accordance with a further exemplary embodiment, component (b) comprises or consists of an ionizable lipid of formula (b-2)

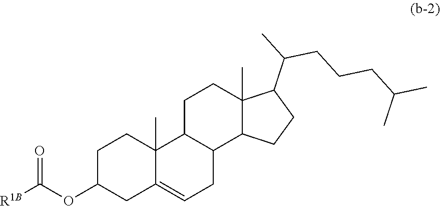

wherein R_{1B} is an organic group comprising one or more primary, secondary or tertiary amino groups,
   or a protonated form thereof, wherein one or more of the nitrogen atoms contained in the primary, secondary or tertiary amino groups comprised by R_{1B} are protonated to provide a compound carrying a positive charge.

Preferably, the compound of formula (b-2) has the following structure:

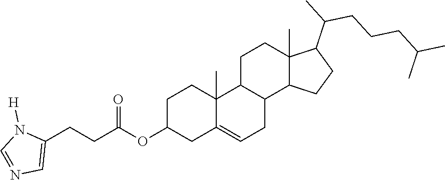 [SMILES: CC(C)CCCC(C)C1CCC2C3CC=C4CC(OC(=O)CCc5cnc[nH]5)CCC4(C)C3CCC12C]

In accordance with another exemplary embodiment, component (b) comprises or consists of an ionizable lipid of formula (b-3)

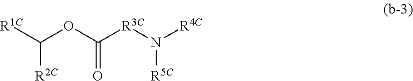

wherein
R_{1C} and R_{2C} are independently selected from a C8-C18 alkyl group and a C8-C18 alkenyl group, preferably from a C12-C18 alkyl group and a C12-C18 alkenyl group,
R_{3C} is a C1-C6 alkanediyl group, preferably a C2 or C3 alkanediyl group, and
R_{4C} and R_{5C} are independently hydrogen or C1-C3 alkyl, and are preferably methyl;
or a protonated form thereof, wherein one or more of the nitrogen atoms contained in the compound of formula (b-3) are protonated to provide a compound carrying a positive charge. As an example of an ionizable lipid of formula (b-3), reference can be made to DLin-MC3-DMA (6Z,9Z,28Z,31Z)-heptatriacont-6,9,28,31-tetraene-19-yl 4-(dimethylamino)butanoate).

In accordance with still another exemplary embodiment, component (b) comprises or consists of an ionizable lipid of formula (b-4)

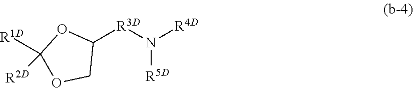

wherein
R_{1D} and R_{2D} are independently selected from a C8-C18 alkyl group and a C8-C18 alkenyl group, preferably from a C12-C18 alkyl group and a C12-C18 alkenyl group,
R_{3D} is a C1-C6 alkanediyl group, preferably a C2 alkanediyl groupy, and
R_{4D} and R_{5D} are independently hydrogen or C1-C3 alkyl, and are preferably methyl;
or a protonated form thereof, wherein one or more of the nitrogen atoms contained in the compound of formula (b-4) are protonated to provide a compound carrying a positive charge.

In accordance with still another exemplary embodiment, component (b) comprises or consists of an ionizable lipidoid of formula (b-5)

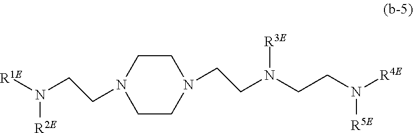

wherein R_{1E} to R_{5E} are independently of each other selected from hydrogen, —CH₂—CH(OH)—R_{7E}, —CH(R_{7E})—CH₂—OH, —CH₂—CH₂—(C═O)—O—R_{7E}, —CH₂—CH₂—(C═O)—NH—R_{7E} and —CH₂—R_{7E} wherein R_{7E} is selected from C3-C18 alkyl or C3-C18 alkenyl having one C—C double bond, provided that at least two residues among R_{1E} to R_{5E} are selected from —CH₂—CH(OH)—R_{7E}, —CH(R_{7E})—CH_{2—}OH, —CH₂—CH₂—(C═O)—O—R_{7E}, —CH₂—CH₂—(C═O)—NH—R_{7E} and —CH₂—R_{7E} wherein R_{7E} is selected from C3-C18 alkyl or C3-C18 alkenyl having one C—C double bond;
   or a protonated form thereof, wherein one or more of the nitrogen atoms contained in the compound of formula (b-5) are protonated to provide a compound carrying a positive charge.

In formula (b-5), R_{1E} to R_{5E} are preferably independently —CH₂—CH(OH)—R_{7E}, wherein R_{7E} is selected from C8-C18 alkyl or C8-C18 alkenyl having one C—C double bond.

Still another exemplary ionizable lipid suitable for use in the present invention which may be comprised in component (b) or of which component (b) may consist is the ionizable lipid disclosed as “cationic lipid of Formula I” in the PCT application WO 2012/000104 A1, starting on page 104 of this document, and including all specific embodiments thereof also discussed in this document.

Further exemplary ionizable lipidoids suitable for use in the present invention which may be comprised in component (b) or of which component (b) may consist are the ionizable lipidoids disclosed and claimed as “aminoalcohol lipidoids” in the PCT application WO 2010/053572 A2, including the compounds of all of the general formulae shown in the summary of the invention on page 4 of the document, and further defined in the remaining application.

Still further exemplary ionizable lipidoids suitable for use in the present invention which may be comprised in component (b) or of which component (b) may consist are the ionizable lipidoids disclosed as amine containing lipidoids of formulae I to V in the PCT application WO 2014/028487 A1, including specific embodiments thereof.

As preferred optional components in addition to the nucleic acid and the ionizable lipid or the ionizable lipidoid, the nanoparticles in the aqueous suspension formulation and in the aerosol of the present invention may comprise one or more of the following components (c1) to (c6):
(c1) a non-ionizable lipid having a sterol structure;
(c2) a phosphoglyceride lipid;
(c3) a PEG-conjugated lipid;
(c4) a polysarcosine-conjugated lipid;
(c5) a PASylated lipid; and
(c6) a cationic polymer.

Component (c1) is a lipid having a sterol structure. As such, suitable lipids are compounds which have a steroid core structure with a hydroxyl group at the 3-position of the A-ring.

An exemplary non-ionizable lipid having a sterol structure which may be comprised by component (c1) or of which component (c1) may consist has a structure of formula (c1-1)

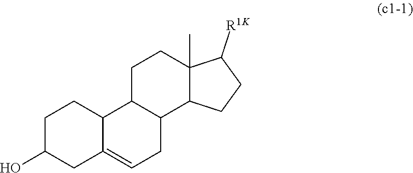

wherein R_{1K} is a C3-C12 alkyl group.

Further exemplary non-ionizable lipids having a sterol structure which may be comprised by component (c1) or of which component (c1) may consist include those disclosed by S. Patel et al., Naturally-occurring cholesterol analogues in lipid nanoparticles induce polymorphic shape and enhance intracellular delivery of mRNA, Nature Communications, 2020, 11:983, in particular those illustrated in FIG. 2 of the publication.

Preferably, component (c1) comprises or consists of cholesterol.

Component (c2) is a phosphoglyceride.

Preferably, component (c2) comprises or consists of a phospholipid selected from a compound of formula (c2-1)

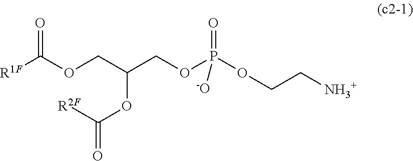

wherein
R_{1F} and R_{2F} are independently selected from a C8-C18 alkyl group and a C8-C18 alkenyl group, preferably from a C12-C18 alkyl group and a C12-C18 alkenyl group, or a pharmaceutically acceptable salt thereof;
   and a phospholipid of formula (c2-2)

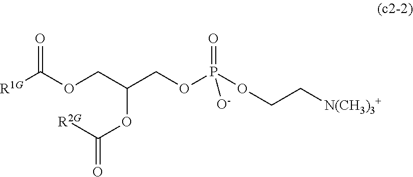

wherein
R_{1G} and R_{2G} are independently selected from a C8-C18 alkyl group and a C8-C18 alkenyl group, preferably from a C12-C18 alkyl group and a C12-C18 alkenyl group,
   or a pharmaceutically acceptable salt thereof.

More preferably, component (c2) comprises or consists of 1,2-dipalmitoyl-sn-glycero-3-phosphocholine (DPPC) or a pharmaceutically acceptable salt thereof.

Exemplary salt forms of the compound of formula (c2-1) include salts formed by the acidic—OH group with a base, or salts formed by the amino group with an acid. As salts formed with a base, mention may be made of alkali metal salts such as sodium or potassium salts; alkaline-earth metal salts such as calcium or magnesium salts and ammonium salts. As exemplary salts formed with an acid, mention may be made of a salt formed with the acidic groups of the nucleic acid, but other salts are not excluded, and mineral acid salts such as chloride, bromide, or iodide, sulfate salts, nitrate salts, phosphate salts, hydrogenphosphate salts, or dihydrogenphosphate salts, carbonate salts, and hydrogencarbonate salts may be mentioned as examples.

Exemplary salt forms of the compound of formula (c2-2) include salts formed by the acidic—OH group attached to the P atom with a base, or salts formed by the quaternary amino group with an anion. As salts formed with a base, mention may be made of alkali metal salts such as sodium or potassium salts; alkaline-earth metal salts such as calcium or magnesium salts and ammonium salts. As exemplary salts formed with anion, mention may be made of a salt formed with the acidic groups of the nucleic acid, but other salts are not excluded, and mineral acid salts such as chloride, bromide, or iodide, sulfate salts, nitrate salts, phosphate salts, hydrogenphosphate salts, or dihydrogenphosphate salts, carbonate salts, and hydrogencarbonate salts may be mentioned as examples.

Component (c3) is a PEG-conjugated lipid, i.e. a lipid which is covalently linked with a polyethylene glycol chain.

Preferably, component (c3) comprises or consists of a PEG-conjugated lipid selected from a compound of formula (c3-1)

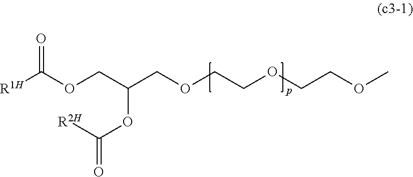

wherein
R_{1H} and R_{2H} are independently selected from a C8-C18 alkyl group and a C8-C18 alkenyl group, preferably from a C12-C18 alkyl group and a C12-C18 alkenyl group, and p is an integer of 5 to 200, preferably 10 to 100, and more preferably 20 to 60;
   and a compound of formula (c3-2)

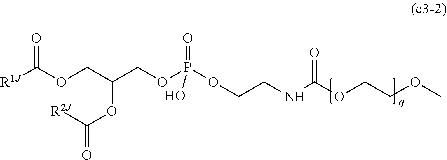

wherein
R_{1J} and R_{2J} are independently selected from a C8-C18 alkyl group and a C8-C18 alkenyl group, preferably from a C12-C18 alkyl group and a C12-C18 alkenyl group, and q is an integer of 5 to 200, preferably 10 to 100, and more preferably 20 to 60 or a pharmaceutically acceptable salt thereof.

Exemplary salt forms of the compound of formula (c3-2) include salts formed by the acidic—OH group attached to the P atom with a base. As salts formed with a base, mention may be made of alkali metal salts such as sodium or potassium salts; alkaline-earth metal salts such as calcium or magnesium salts and ammonium salts.

More preferably, component (c3) comprises or consists of 1,2-dimyristoyl-sn-glycerolmethoxy(polyethylene glycol) (DMG-PEG), and still more preferably component d) comprises or consists of 1,2-dimyristoyl-sn-glycerolmethoxy(polyethylene glycol)-2000 (DMG-PEG2k).

Component (c4) is a polysarcosine-conjugated lipid, i.e. a lipid which is covalently linked with a polymeric moiety of the formula (c4-1):

—[C(O)—CH₂—N(CH₃)]ᵣ_{^{−}} (c4-1)
wherein r denotes the number of repeating units, and is preferably 10 to 100.

Component (c5) is a PASylated lipid, i.e. a lipid which is covalently linked with a polymeric moiety formed by proline (pro)/alanine (ala)/serine (ser) repetitive residues.

Component (c6) is a cationic polymer. Such polymers suitable for use in the formation of nanoparticles comprising a nucleic acid are known in the art. Exemplary suitable cationic polymers are discussed in A. C. Silva et al., Current Drug Metabolism, 16, 2015, 3-16, and in the literature referred to therein, in J. C. Kasper et al., J. Contr. Rel. 151 (2011), 246-255, in WO 2014/207231 and in the literature referred to therein, and in WO 2016/097377 and in the literature referred to therein.

Suitable cationic oligomers or polymers include in particular cationic polymers comprising a plurality of units wherein an amino group is contained. The amino groups may be protonated to provide the cationic charge of the polymer.

Polymers are preferred which comprise a plurality of units independently selected from the following (1), (2), (3) and (4):

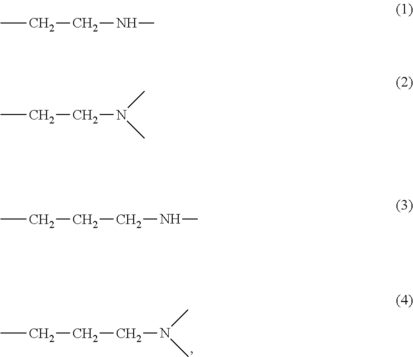

wherein one or more of the nitrogen atoms of the repeating units (1), (2), (3) and/or (4) may be protonated to provide the cationic charge of the polymer.

Particularly preferred as cationic polymers are the following four classes of polymers comprising a plurality of units wherein an amino group is contained.

As the first preferred class, poly(ethylene imine) (“PEI”) is mentioned, including branched poly(ethylene imine) (“brPEI”).

The second preferred class of cationic polymers are polymers comprising a plurality of groups of the following formula (c6-1) as a side chain and/or as a terminal group, as they are disclosed as groups of formula (II) in WO 2014/207231 (applicant ethris GmbH):

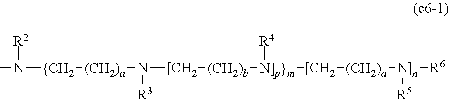

wherein the variables a, b, p, m, n and R₂ to R₆ are defined as follows, independently for each group of formula (c6-1) in a plurality of such groups:
a is 1 and b is an integer of 2 to 4; or a is an integer of 2 to 4 and b is 1,
p is 1 or 2,
m is 1 or 2; n is 0 or 1 and m+n is≥2; and
R₂ to R₅ are, independently of each other, selected from hydrogen; a group —CH₂—CH(OH)—R₇, —CH(R₇)—CH₂—OH, —CH₂—CH₂—(C═O)—O—R₇, —CH₂—CH₂—(C═O)—NH—R₇ or —CH₂—R₇ wherein R₇ is selected from C3-C18 alkyl or C3-C18 alkenyl having one C—C double bond; a protecting group for an amino group; and a poly(ethylene glycol) chain;
R₆ is selected from hydrogen; a group —CH₂—CH(OH)—R₇, —CH(R₇)—CH—OH, —CH₂—CH₂—(C═O)—O—R₇, —CH₂—CH₂—(C═O)—NH—R₇ or —CH₂—R₇ wherein R₇ is selected from C3-C18 alkyl or C3-C18 alkenyl having one C—C double bond; a protecting group for an amino group; —C(NH)—NH₂; a poly(ethylene glycol) chain; and a receptor ligand,
and wherein one or more of the nitrogen atoms indicated in formula (c6-1) may be protonated to provide a cationic group of formula (c6-1).

As regards further preferred definitions of these polymers, and of the variables contained in formula (c6-1) above, the respective disclosure in WO 2014/207231 with regard to its groups of formula (II) also applies for the invention described herein.

The third preferred class of cationic polymers are polymers comprising a plurality of groups of the following formula (c6-2) as repeating units, as they are disclosed as groups of formula (III) in WO 2014/207231 (applicant ethris GmbH):

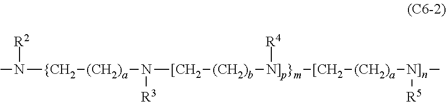

wherein the variables a, b, p, m, n and R₂ to R₅ are defined as follows, independently for each group of formula (c6-2) in a plurality of such groups:
a is 1 and b is an integer of 2 to 4; or a is an integer of 2 to 4 and b is 1,
p is 1 or 2,
m is 1 or 2; n is 0 or 1 and m+n is≥2; and
R₂ to R₅ are, independently of each other, selected from hydrogen; a group —CH_{2—}CH(OH)—R₇, —CH(R₇)—CH₂—OH, —CH₂—CH₂—(C═O)—O—R₇, —CH₂—CH₂—(C═O)—NH—R₇ or —CH₂—R₇, wherein R₇ is selected from C3-C18 alkyl or C3-C18 alkenyl having one C—C double bond; a protecting group for an amino group; —C(NH)—NH₂; and a poly(ethylene glycol) chain;
and wherein one or more of the nitrogen atoms indicated in formula (c6-2) may be protonated to provide a cationic group of formula (c6-2).

As regards further preferred definitions of these polymers, and of the variables contained in formula (c6-2) above, the respective disclosure in WO 2014/207231 with regard to its repeating units of formula (III) also applies for the invention described herein.

The fourth preferred class of cationic polymers is provided by a statistical copolymer as it is disclosed in WO 2016/097377 (applicant ethris GmbH). It comprises a plurality of repeating units (a) independently selected from repeating units of the following formulae (a1) and (a2):

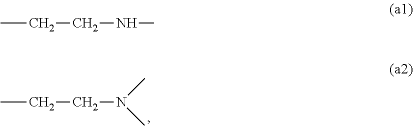

and a plurality of repeating units (b) independently selected from repeating units of the following formulae (b1) to (b4):

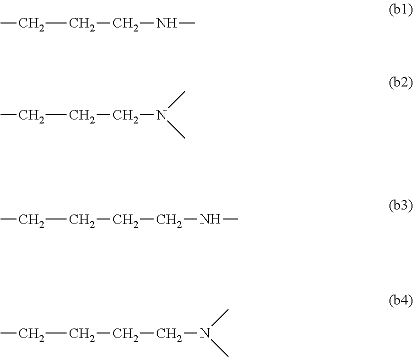

and the molar ratio of the sum of the repeating units (a) to the sum of the repeating units (b) lies within the range of 0.7/1.0 to 1.0/0.7, and one or more of the nitrogen atoms of the repeating units (a) and/or (b) contained in the copolymer may be protonated to provide a cationic copolymer.

As regards further preferred definitions of this copolymer, the respective disclosure in WO 2016/097377 also applies for the invention described herein. As noted therein, a particularly preferred copolymer is a linear copolymer which comprises repeating units (a1) and (b1), or which consists of repeating units (a1) and (b1).

As an optional component of the nanoparticles, a polyanion component which is different from the nucleic acid may also be comprised. Examples of such a polyanion are polyglutamic acid and chondroitin sulfate. If such a polyanion component different from the nucleic acid is used in the nanoparticles, its amount is preferably limited such that the amount of anionic charges provided by the polyanion component is not higher than the amount of the anionic charges provided by the nucleic acid.

As explained above, the lipid or lipidoid nanoparticles which are present in the suspension formulation and in the aerosol in accordance with the invention comprise (a) a nucleic acid and (b) an ionizable lipid or an ionizable lipidoid. If a lipidoid is comprised, the nanoparticles shall be referred to herein as lipidoid nanoparticles.

Preferably, the nanoparticles comprise, more preferably consist of,
the nucleic acid (a),
the ionizable lipid or ionizable lipidoid (b),
and optionally one or more of
the non-ionizable lipid having a sterol structure (c1);
the phosphoglyceride lipid (c2);
the PEG-conjugated lipid (c3);
the polysarcosine-conjugated lipid (c4);
the PASylated lipid (c5); and
the cationic polymer (c6).

Exemplary suspension formulations comprising nanoparticles formed from the components listed above, which are also suitable for use in the context of the present invention, include those disclosed by S. Patel et al., Naturally-occurring cholesterol analogues in lipid nanoparticles induce polymorphic shape and enhance intracellular delivery of mRNA, Nature Communications, 2020, 11:983.

It will be understood that components of the nanoparticles, and in particular components (a) and (b), and optionally one or more of (c1) to (c6), are typically contained as a mixture in the nanoparticles.

In terms of the amounts of these components, it is further preferred that the nanoparticles comprise, more preferably consist of:
the nucleic acid, and
30 to 65 mol % of the ionizable lipid or ionizable lipidoid (b),
and one or more of the following components:
10 to 50 mol % of the lipid having a sterol structure (c1),
4 to 50 mol % of the phosphoglyceride lipid (c2),
0.5 to 10 mol % of one of the PEG-conjugated lipid (c3), the polysarcosine-conjugated lipid (c4) and the PASylated lipid (c5), or of any combination thereof,
0.5 to 10 mol % of the cationic polymer (c6),
such that the sum of (b) and (c1) to (c6) amounts to 100 mol %. As will be understood, the molar percentages for components (c1) to (c6) are indicated with the proviso that not all of these components need to be present in the nanoparticles. Thus, for example, the cationic polymer can be present or absent in the context of this preferred embodiment, but if it is present, it is used in the amount of 0.5 to 10 mol %. As further indicated above, the amount of component(s) (c1), (c2), (c3), (c4), (c5) and/or (c6) in the context of this preferred embodiment is such that the sum of (b) and (c1) to (c6) amounts to 100 mol %.

It is still further preferred that the nanoparticles comprise, or consist of
the nucleic acid (a),
the ionizable lipid or ionizable lipidoid (b),
the non-ionizable lipid having a sterol structure (c1),
the phosphoglyceride lipid (c2), and
the PEG-conjugated lipid (c3).

In terms of the amounts of these components, it is still further preferred that the nanoparticles comprise, more preferably consist of:
the nucleic acid (a),
30 to 65 mol % of the ionizable lipid or ionizable lipidoid (b),
10 to 50 mol % of the lipid having a sterol structure (c1),
4 to 50 mol % of the phosphoglyceride lipid (c2), and
0.5 to 10 mol % of the PEG-conjugated lipid (c3),
such that the sum of (b) and (c1) to (c3) amounts to 100 mol %.

In line with the above information related to preferred nucleic acids and related to the preferred components of the lipid composition other than the nucleic acid, the lipid nanoparticles contained in the suspension formulation in accordance with the invention and in the aerosol in accordance with the invention, respectively, preferably comprise
(a) mRNA as a nucleic acid;
(b) an ionizable lipidoid of formula (b-1b)

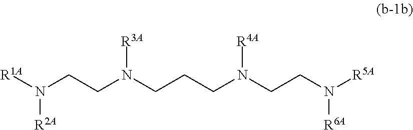

wherein R_{1A} to R_{6A} are independently selected from hydrogen and —CH₂—CH(OH)—R_{7A}, wherein R_{7A} is selected from C8-C18 alkyl and C8-C18 alkenyl having one C—C double bond, provided that at least two residues among R_{1A} to R_{6A} are —CH₂—CH(OH)—R_{7A}, more preferably at least four residues among R_{1A} to R_{6A} are —CH₂—CH(OH)—R_{7A}, wherein R_{7A} is selected from C8-C18 alkyl and C8-C18 alkenyl having one C—C double bond;
or a protonated form thereof wherein one or more of the nitrogen atoms indicated in formula (b-1b) are protonated to provide a cationic lipidoid;
(c1) a non-ionizable lipid having a sterol structure of formula (c1-1)

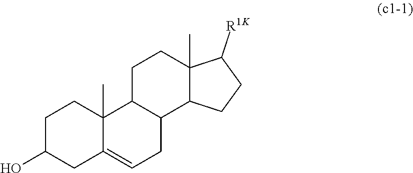

wherein R_{1K} is a C3-C12 alkyl group;
(c2) a phosphoglyceride of formula (c2-2)

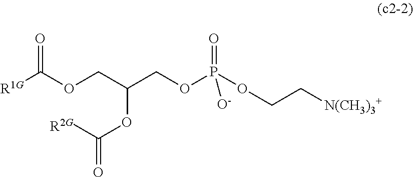

wherein R_{1G} and R₂G are independently selected from a C8-C18 alkyl group and a C8-C18 alkenyl group, preferably from a C12-C18 alkyl group and a C12-C18 alkenyl group,
or a pharmaceutically acceptable salt thereof; and
(c3) a PEG conjugated lipid of formula (c3-1)

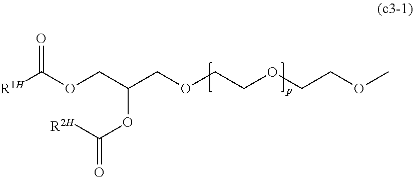

wherein R_{1H} and R_{2H} are independently selected from a C8-C18 alkyl group and a C8-C18 alkenyl group, preferably from a C12-C18 alkyl group and a C12-C18 alkenyl group, and p is an integer of 5 to 200, preferably 10 to 100, and more preferably 20 to 60.

In the nanoparticles contained in the suspension formulation and in the aerosol in accordance with the invention, the composition of the nanoparticles is preferably such that the weight ratio in the nanoparticles of the sum of the weights of components other than the nucleic acid to the weight of the nucleic acid is in the range of 30:1 to 1:1, more preferably 20:1 to 2:1 and most preferably 15:1 to 3:1.

The N/P ratio, i.e. the ratio of the number of amine nitrogen atoms provided by the ionizable lipid or the ionizable lipidoid to the number of phosphate groups provided by the nucleic acid of the nanoparticles is preferably in the range of 0.5 to 20, more preferably in the range of 0.5 to 10.

The lipid or lipidoid nanoparticles contained in the suspension formulation and in the aerosol in accordance with the invention preferably have a Z-average diameter in the range of 10 to 500 nm, more preferably in the range of 10 to 250 nm, still more preferably 20 to 200 nm. The indicated particle diameter is the hydrodynamic diameter of the particles, as determined by dynamic light scattering (DLS). Measurements are generally carried out at 25° C.

The polydispersity index of the nanoparticles contained in the suspension formulation and in the aerosol in accordance with the invention is preferably in the range of 0.05 to 0.4, more preferably in the range of 0.05 to 0.2. The polydispersity index can be determined by dynamic light scattering (DLS). Measurements are generally carried out at 25° C.

It is possible to provide a suspension formulation or an aerosol containing different lipid or lipidoid nanoparticles as defined above, i.e. particles which differ in terms of their components. However, preferably the nanoparticles contained in the suspension formulation in accordance with the invention or in the aerosol in accordance with the invention are composed of the same components.

The lipid nanoparticles can be conveniently prepared by mixing a solution containing the nucleic acid, e.g. in an aqueous solvent containing a buffer, such as a citrate buffer with a pH of 4.5, and optionally containing a salt such as sodium chloride, and a solution containing the ionizable lipid or ionizable lipidoid, e.g. in ethanol. Further optional components can be incorporated e.g. by adding them to one of the two solutions. The lipid nanoparticles generated in this manner can be further processed by chromatography and/or dialysis and/or tangential flow filtration in order to obtain the lipid nanoparticles in a desired liquid composition. Before or during these downstream processing steps, further excipients such as cryoprotectants and other excipients can be added to obtain a desired pharmaceutical composition. If the nanoparticles are subjected to tangential flow filtration, it is preferred for stability reasons to carry out the filtration on a suspension of the nanoparticles comprising the triblock copolymer which contains one poly(propylene oxide) block and two poly(ethylene oxide) blocks as defined as a component of the vehicle solution herein.

To that extent, the invention further provides a method for the preparation of the aqueous suspension formulation for aerosol formation comprising lipid or lipidoid nanoparticles which are suspended in an aqueous vehicle solution, said method comprising a step of mixing a solution containing the nucleic acid (a), and a solution containing the ionizable lipid or ionizable lipidoid (b), to form a suspension comprising the lipid or lipidoid nanoparticles. Further components, such as one or more of components (c1) to (c6) can be conveniently incorporated into the nanoparticles e.g. by adding them to the solution containing the ionizable lipid or the ionizable lipidoid.

As a preferred embodiment, the invention provides a method for the preparation of the aqueous suspension formulation for aerosol formation comprising lipid or lipidoid nanoparticles which are suspended in an aqueous vehicle solution, said method comprising
a step of mixing a solution containing the nucleic acid (a), and a solution containing the ionizable lipid or ionizable lipidoid (b), to form a suspension comprising the lipid or lipidoid nanoparticles;
a step of adding the triblock copolymer which contains one poly(propylene oxide) block and two poly(ethylene oxide) blocks as defined herein to the suspension; and
a step of subjecting the suspension to tangential flow filtration to yield the aqueous suspension formulation in accordance with the invention.

The aqueous suspension formulation for aerosol formation comprises the lipid or lipidoid nanoparticles discussed above together with an aqueous vehicle solution. As indicated by the reference to a suspension formulation, the nanoparticles are suspended in the vehicle solution.

The vehicle solution is an aqueous solution, i.e. a solution wherein the main solvent, in terms of the total volume of solvent(s), is water, preferably a solution containing more than 70% of water, more preferably more than 90% of water, as a solvent, indicated as the volume percentage of water in the total volume of solvent(s) contained in the vehicle solution (at a temperature of 25° C.). Most preferably, water is the only solvent in the vehicle solution. Thus, the vehicle solution is a liquid at room temperature (e.g. 25° C.).

The weight per volume ratio of the nanoparticles in the vehicle solution in the composition is preferably in the range 0.5 g/L to 100 g/L, preferably 10 g/L to 100 g/L, more preferably 10 g/L to 50 g/L and most preferably 10 g/L to 75 g/L.

The concentration of the nucleic acid, provided by the lipid or lipidoid nanoparticles, in the suspension formulation preferably ranges from 0.01 to 10 mg/ml, more preferably from 0.02 to 5 mg/ml, and most preferably from 0.1 to 5 mg/ml, based on the total volume of the suspension formulation.

As noted above, the lipid or lipidoid nanoparticles contained in the suspension formulation and in the aerosol in accordance with the invention preferably have a Z-average diameter in the range of 10 to 500 nm, more preferably in the range of 10 to 250 nm, still more preferably 20 to 200 nm. The indicated particle diameter is the hydrodynamic diameter of the particles, as determined by dynamic light scattering (DLS). Measurements are generally carried out at 25° C.

The polydispersity index of the nanoparticles contained in the suspension formulation and in the aerosol in accordance with the invention is preferably in the range of 0.05 to 0.4, more preferably in the range of 0.05 to 0.2. The polydispersity index can be determined by dynamic light scattering (DLS). Measurements are generally carried out at 25° C.

The vehicle solution comprises a triblock copolymer which contains one poly(propylene oxide) block and two poly(ethylene oxide) blocks.

Preferably, the triblock copolymer is an A-B-A triblock copolymer which contains one poly(propylene oxide) block B of formula (p-1):

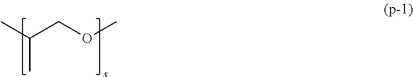

wherein s is an integer of 15 to 67, preferably 20 to 40, and
two poly(ethylene oxides) blocks A of formula (p-2):

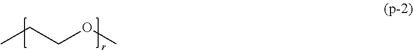

wherein r is, independently for each block, an integer of 2 to 130, preferably 50 to 100, and more preferably 60 to 90.

More preferably, the triblock copolymer has the following structure:

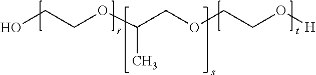

wherein r and t are independently of each other integers of 2 to 130, preferably 50 to 100, and more preferably 60 to 90,
and s is an integer of 15 to 67, preferably 20 to 40.

Most preferably, Poloxamer P188 is used as the triblock copolymer.

The vehicle solution generally comprises the triblock copolymer dissolved therein. However, as will be appreciated by the skilled reader, this does not exclude the possibility that a certain amount of the copolymer molecules is adsorbed to the lipid or lipidoid nanoparticles which are contained in the composition.

Preferably, the composition for aerosol formation comprises the triblock copolymer at a concentration of 0.05 to 5% w/v (i.e. gram per 100 mL) preferably 0.1 to 2%, based on the total volume of the composition.

In addition to the triblock copolymer, other excipients may be present in the vehicle solution. Preferably, the vehicle solution further comprises at least one of sucrose and NaCl, more preferably sucrose and NaCl.

The suspension formulation in accordance with the invention can be conveniently prepared e.g. by a method including adding the triblock copolymer to a suspension comprising a vehicle solution and the lipid or lipidoid nanoparticles, or including adding the lipid or lipidoid nanoparticles to a vehicle solution comprising the triblock copolymer.

The aqueous suspension formulation for aerosol formation in accordance with the present invention can be nebulized to provide the aerosol in accordance with the invention. Advantageously, a negative influence of the nebulization step on the nanoparticles and the nucleic acid contained in the aqueous suspension formulation can be minimized or even avoided in this manner. Moreover, the nebulization can be accomplished in an efficient manner within a reasonable period of time of e.g. 60 minutes or less, preferably 30 min or less, for a given dose of mRNA.

Thus, the aerosol which is obtainable by nebulization of the aqueous suspension formulation for aerosol formation in accordance with the invention comprises aerosol droplets dispersed in a gas phase. The aerosol droplets comprise the lipid or lipidoid nanoparticles as discussed above, including any preferred embodiments thereof, and an aqueous vehicle solution for the nanoparticles. The aqueous vehicle solution comprises the triblock copolymer which contains one poly(propylene oxide) block and two poly(ethylene oxide) blocks that is provided by the vehicle solution of the aqueous suspension formulation of the invention, and is discussed in this context above.

As explained above, it has been found that the presence of the triblock copolymer allows the favorable nanoparticle characteristics to be retained which are exhibited by the nanoparticles of the aqueous suspension formulation discussed above prior to nebulization.

Thus, the lipid or lipidoid nanoparticles contained in the aerosol droplets of the aerosol in accordance with the invention preferably have a Z-average diameter in the range of 10 to 500 nm, more preferably in the range of 10 to 250 nm, still more preferably 20 to 200 nm. The indicated particle diameter is the hydrodynamic diameter of the particles, as determined by dynamic light scattering (DLS). Measurements are generally carried out at 25° C.

The polydispersity index of the lipid or lipidoid nanoparticles contained in the aerosol droplets of the aerosol in accordance with the invention is preferably in the range of 0.05 to 0.4, more preferably in the range of 0.05 to 0.2. The polydispersity index can be determined by dynamic light scattering (DLS). Measurements are generally carried out at 25° C.

The vehicle solution in the aerosol droplets of the aerosol which is derived from the suspension formulation is an aqueous solution, i.e. a solution wherein the main solvent, in terms of the total volume of solvent(s), is water. Preferably, the vehicle solution contains more than 70% of water, more preferably more than 90% of water, as a solvent, indicated as the volume percentage of water in the total volume of solvent(s) contained in the vehicle solution (at a temperature of 25° C.). Most preferably, water is the only solvent in the vehicle solution.

As noted above, the aerosol in accordance with the invention comprises droplets dispersed in a gas phase, typically dispersed in air. The droplets are obtainable via the nebulization of the composition for aerosol formation in accordance with the invention. They comprise a liquid phase which is derived from the vehicle solution of the composition described in detail above, and the lipid or lipidoid nanoparticles. Typically, the lipid or lipidoid nanoparticles are dispersed in the vehicle solution. Moreover, the aerosol droplets typically comprise a plurality of the lipid or lipidoid nanoparticles dispersed in a single droplet.

As further explained above, the aerosol in accordance with the invention can be administered to a subject, in particular to or via the respiratory tract of the subject, preferably via pulmonary administration or nasal administration. Typically, the administration is accomplished via inhalation of the aerosol by the subject.

Aerosol droplets can be characterized via their aerodynamic diameter, which takes into account their density and their shape. The aerodynamic diameter is defined as the diameter of a spherical particle or droplet with a density of 1 g/cm³, which has the same sinking speed in air as the droplet under consideration (Luftbeschaffenheit-Festlegung von Partikelgröβenverteilungen für die gesundheitsbezogene Schwebstaubprobenahme, (1995); Vincent J H. Aerosol Sampling—Science, Standards, Instrumentation and Applications. Chichester, England: John Wiley & Sons, Ltd.; 2007). Size distributions of the aerodynamic diameter are often parameterized via the Mass Median Aerodynamic Diameter (MMAD), i.e. the median mass-related aerodynamic diameter. The MMAD is thus the diameter at which particles smaller or larger than this value each contribute 50% of the total mass and thus a measure of the average size of a particle. The MMAD can be measured with a cascade impactor or a next generation impactor (Preparations for inhalation: Aerodynamic assessment of fine particles; European Pharmacopoeia 90; Volume I: EDQM Council of Europe; 2019). The mass median aerodynamic diameter (MMAD) of an aerosol droplet has an impact on where in the respiratory tract an aerosol particle will deposit. While particles with an MMAD of 10 μm or more tend to be already deposited (impacted) at the throat due to their inertia, particles between 0.1 μm and 1.0 μm tend to be too light and may be exhaled again due to diffusion processes caused by Brownian motion.

The aerosol droplets of the aerosol in accordance with the invention preferably have an MMAD, as determined by measurement using a cascade impactor or a next generation impactor of 2 to 10 μm, more preferably 3 to 8 μm.

Nebulization devices (nebulizers) for forming an aerosol from a suspension formulation comprising particles contained in a vehicle solution are known in the art and are commercially available. A nebulizer is an instrument that converts a liquid into a mist of fine droplets dispersed in a gas phase, i.e. an aerosol, which is suitable for inhalation. Examples of suitable nebulizers for generation of aerosol which can be used, among others, in the context of the invention are:
a jet nebulizer, e.g. Pari Boy (Pari);
a vibrating mesh nebulizer, e.g. Pari eFlow (Pari), Aeroneb (Aerogen), Fox (Vectura), or Innospire GO (Philips);
a passive mesh nebulizer, e.g. MicroAir U22 (Omron), or Smarty (Flaem);
an ultrasonic nebulizer, e.g. My-520A (Fish), or Aerosonic Combineb (Flores)
a soft mist inhaler e.g. Trachospray (MedSpray), Pulmospray (Medspray), or Respimat (Boehriner Ingelheim).

In the context of the invention, the suspension formulation for aerosol formation is preferably nebulized using a vibrating mesh nebulizer or a soft mist inhaler, more preferably a soft mist inhaler.

In still a further aspect, the invention provides a method for the preparation of the aerosol in accordance with the invention as discussed above, said method comprising a step of nebulizing the suspension formulation for aerosol formation in accordance with the invention.

It has been found that the suspension formulation in accordance with the invention can be effectively and continuously nebulized over extended periods of time without a loss of quality of the nanoparticles contained in the suspension formulation and in the aerosol droplets (e.g. by aggregation of the particles). Thus, effective doses of the nucleic acid as the active agent contained in the nanoparticles can be provided and administered in the form of an aerosol in a reasonable amount of time, such as 60 minutes or less, preferably 30 minutes or less.

The nucleic acid, such as RNA, preferably mRNA, which is present in the lipid or lipidoid nanoparticles used in the context of the present invention is particularly useful in a medical setting and in the treatment of diseases and disorders, in particular in nucleic acid-based therapies. Thus, the suspension formulation for aerosol formation and the aerosol in accordance with the invention are generally provided as or used as a medicament or as a pharmaceutical composition.

In particular, the suspension formulation for aerosol formation and the aerosol in accordance with the present invention are suitable for administration to a subject. In this manner, the nucleic acid such as RNA, preferably the mRNA, contained in the nanoparticles of the suspension formulation and the aerosol can also be administered to the subject. A preferred route of administration for the composition is the administration of the aerosol provided by nebulization of the suspension formulation in accordance with the invention to or via the respiratory tract, in particular the pulmonary administration or nasal administration. Typically, the aerosol is inhaled by the subject to which it is administered.

Via administration to a subject, the nucleic acid contained in the lipid or lipidoid nanoparticles particles may be delivered to target cells in or via the respiratory tract. The term “delivered to target cells” preferably means transfer of the nucleic acid into the cell.

Thus, the present invention also provides the aqueous suspension formulation for use as a medicament, wherein the suspension formulation is to be nebulized and the aerosol provided by the nebulization is to be administered to a subject. Likewise, the invention provides the aerosol in accordance with the present invention for use as a medicament.

The aqueous suspension formulation or the aerosol can be administered to the subject at a suitable dose. The dosage regimen will be determined by the attending physician and clinical factors. As is well known in the medical arts, dosages for any one subject depend upon many factors, including the subject's size, body surface area, age, the particular compound to be administered, sex, time and route of administration, general health, and other drugs being administered concurrently. A typical dose of therapeutically active substances can be, for example, in the range of 1 ng to several grams. The dosage of a nucleic acid for expression or for inhibition of expression should correspond to this range; however, doses below or above this exemplary range are envisioned, especially considering the aforementioned factors. Generally, the regimen as a regular administration of the pharmaceutical composition should be in the range of 0.01 μg to 10 mg units per kilogram of body weight per day. Progress can be monitored by periodic assessment. Dosages will vary but a preferred dosage for administration of nucleic acids as constituents of the composition of the present invention is from approximately 10¹⁰ to 10¹⁹ copies of the nucleic acid molecule.

Also made available by the present invention is a method of treatment, comprising the nebulization of the aqueous suspension formulation in accordance with the invention, and the administration of the aerosol provided by nebulization to or via the respiratory tract of a subject, preferably via pulmonary administration or nasal administration. Thus, the nucleic acid contained in said suspension formulation can cause a preventive or therapeutic effect. Notably, the term “subject” comprises animals and humans. Likewise, the invention provides a method of treatment which comprises the administration of the aerosol according to the invention to a subject to or via the respiratory tract of a subject, preferably via pulmonary administration or nasal administration. As noted above, the aerosol is typically inhaled by the subject to which it is administered.

By administering the aqueous suspension formulation or the aerosol of the present invention to a subject, diseases or disorders can be treated or prevented. The term “disease” refers to any conceivable pathological condition that can be treated, prevented or vaccinated against by employing the aqueous suspension formulation or the aerosol of the present invention. Preferably, the disease to be treated or prevented is a pulmonary disease. Said diseases may e.g. be inherited, acquired, infectious or non-infectious, age-related, cardiovascular, metabolic, intestinal, neoplastic (in particular cancer) or genetic. A disease can be based, for example, on irregularities of physiological processes, molecular processes, biochemical reactions within an organism that in turn can be based, for instance, on the genetic equipment of an organism, on behavioural, social or environmental factors such as the exposure to chemicals or radiation.

Thus, the invention further provides the aqueous suspension formulation of the present invention for use in the treatment or prevention of a disease or disorder via a nucleic acid-based therapy, wherein the treatment or prevention comprises the nebulization of the suspension formulation and the administration of the aerosol provided by nebulization to or via the respiratory tract, preferably via pulmonary administration or nasal administration. Likewise, the invention provides the aerosol of the present invention, for use in the treatment or prevention of a disease or disorder via a nucleic acid-based therapy, wherein the treatment or prevention comprises the administration of the aerosol to or via the respiratory tract, preferably via pulmonary administration or nasal administration. As noted above, the aerosol is typically inhaled by the subject to which it is administered.

In a preferred embodiment, the invention further provides the aqueous suspension formulation of the present invention for use in the treatment or prevention of a pulmonary disease, wherein the treatment or prevention comprises the nebulization of the suspension formulation and the administration of the aerosol provided by nebulization to or via the respiratory tract, preferably via pulmonary administration or nasal administration. Likewise, the invention provides the aerosol as disclosed above, including its preferred embodiments, for use in the treatment or prevention of a pulmonary disease, wherein the treatment or prevention comprises the administration of the aerosol to or via the respiratory tract, preferably via pulmonary administration or nasal administration. As noted above, the aerosol is typically inhaled by the subject to which it is administered.

The terms “treatment” or “treating” used herein generally mean obtaining a desired pharmacological and/or physiological effect in the human or animal body. Accordingly, the treatment of the present invention may relate to the treatment of (acute) states of a certain disease but may also relate to the prophylactic treatment in terms of completely or partially preventing a disease or symptom thereof. Preferably, the term “treatment” is to be understood as being therapeutic in terms of partially or completely curing a disease and/or adverse effects and/or symptoms attributed to the disease. “Acute” in this respect means that the subject shows symptoms of the disease. In other words, the subject to be treated is in actual need of a treatment and the term “acute treatment” in the context of the present invention relates to the measures taken to actually treat the disease after the onset of the disease or the breakout of the disease. The treatment may also be prophylactic or preventive treatment, i.e., measures taken for disease prevention, e.g., in order to prevent the infection and/or the onset of the disease. Therapeutic progress can be monitored by periodic assessment.

Generally, the nucleic acid is included in an effective amount in the suspension formulation and the aerosol in accordance with the present invention. The term “effective amount” refers to an amount sufficient to induce a detectable therapeutic response in the subject to which the pharmaceutical composition is to be administered. In accordance with the above, the content of the nucleic acid is not limited as far as it is useful for treatment as described above. As noted above, the composition for aerosol formation or the aerosol, wherein the particles comprising the nucleic acid are contained, preferably comprises the particles in an amount so as to provide the nucleic acid contained in the particles at a concentration of 0.01 to 50 mg/ml, more preferably 0.02 to 30 mg/ml, and most preferably 0.05 to 10 mg/ml, based on the total volume of the composition.

Exemplary subjects include a mammal such as a dog, cat, pig, cow, sheep, horse, rodent, e.g., rat, mouse, and guinea pig, or a primate, e.g., gorilla, chimpanzee, and human. In a most preferable embodiment, the subject is a human.

As noted above, the suspensions formulation and the aerosol in accordance with the present invention may be for use in the treatment or the prevention via a nucleic acid-based therapy. For example, the nucleic acid-based therapy is for the treatment or prevention of a disease or disorder as recited in the above Table A.

The suspension formulation and the aerosol of the present invention are particularly suitable for use in the treatment or prevention of a pulmonary disease. As exemplary diseases, asthma, surfactant metabolism dysfunction, surfactant protein B (SPB) deficiency, ATP-binding cassette sub-family A member 3 (ABCA3) deficiency, cystic fibrosis, alpha-1 antitrypsin (A1AT) deficiency; lung cancer, surfactant protein C (SPC) deficiency, alveolar proteinosis, sarcoidosis, acute and chronic bronchitis, emphysema, McLeod-Syndrom, chronic obstructive pulmonary disease (COPD) , asthma bronchiale, bronchiectasis, pneumoconiosis, asbestosis, Acute Respiratory Distress Syndrome (ARDS), Infant respiratory distress syndrome (IRDS), pulmonary oedema, pulmonary eosinophilia, Loffler's pneumonia, Hamman-Rich syndrome, idiopathic pulmonary fibrosis, interstitial pulmonary diseases, primary ciliary dyskinesia, pulmonary arterial hypertension (PAH) and STAT5b deficiency, clotting defects, especially hemophilia A and B; complement defects, especially protein C deficiency, thrombotic thrombocytopenic purpura and congenital hemochromatosis, especially Hepcidin deficiency; pulmonary infectious diseases, preferably respiratory syncytial virus (RSV) infection, parainfluenza virus (PIV) infection, influenza virus infection, rhinoviruses infection, severe acute respiratory syndrome, corona virus (SARS-CoV) infection, tuberculosis, Pseudomonas aeruginosa infection, Burkholderia cepacia infection, Methicillin-Resistant Staphylococcus aureus (MRSA) infection, and Haemophilus influenzae infection may be mentioned.

However, it will be understood that the aqueous suspension formulation for aerosol formulation may translocate from a respiratory tissue to other tissues or organs in the body and may transfect cells in said distant tissues or organs. Similarly, the protein(s) encoded by the mRNA comprised in the suspension formulation for aerosol formulation may translocate from a respiratory tissue to other tissues or organs in the body and may have a therapeutic effect in said distant tissues or organs.

In other exemplary embodiments, the composition and the aerosol of the present invention may be for use in nucleic acid-based therapies in the treatment or prevention of lysosomal diseases like Gaucher disease, Fabry disease, MPS I, MPS II (Hunter syndrome), MPS VI and Glycogen storage diseases such as for example Glycogen storage disease type I (von Gierecke's disease), type II (Pompe's disease), type III (Cori's disease, type IV (Andersen's disease, type V (McArdle's disease, type VI (Hers disease), type VII (Tauri's disease), type VII, type IX, type X, type XI (Fanconi-Bickel syndrome), type XI, or type 0. Transcript replacement therapies/enzyme replacement therapies beneficially do not affect the underlying genetic defect, but increase the concentration of the enzyme in which the subject is deficient. As an example, in Pompe's disease, the transcript replacement therapy/enzyme replacement therapy replaces the deficient Lysosomal enzyme acid alpha-glucosidase (GAA).

In accordance with further examples, nucleic acid-based therapies in accordance with the present invention may be for use in treating cancer, a cardiovascular disease, a viral infection, an immune dysfunction, an autoimmune disease, a neurologic disorder, an inherited metabolic disorder or a genetic disorder or any disease where a protein or protein fragment produced in a cell may have a beneficial effect for the patent. Examples of cancer include head and neck cancer, breast cancer, renal cancer, bladder cancer, lung cancer, prostate cancer, bone cancer, brain cancer, cervical cancer, anal cancer, colon cancer, colorectal cancer, appendix cancer, eye cancer, gastric cancer, leukemia, lymphoma, liver cancer, skin cancer, ovarian cancer, penile cancer, pancreatic cancer, testicular cancer, thyroid cancer, vaginal cancer, vulvar cancer, endometrial cancer, cardiac cancer and sarcoma. Examples of cardiovascular diseases include atherosclerosis, coronary heart disease, pulmonary heart disease and cardiomyopathy. Examples of immune dysfunctions and autoimmune diseases include, but are not limited to, rheumatic diseases, multiple sclerosis and asthma. Examples of viral infections include, but are not limited to, infections with human immunodeficiency virus, herpes simplex virus, human papillomavirus as well as hepatitis B and C virus. Examples of neurologic disorders include, but are not limited to, Parkinson's disease, multiple sclerosis, and dementia. Examples of inherited metabolic disorders include, but are not limited to, Gaucher's disease and Phenylketonuria.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### EXAMPLES

| Abbreviation | Description |
|---|---|
| LNP | lipid nanoparticle or lipidoid nanoparticle |
| mRNA | messenger ribonucleic acid |
| N/P | carrier amine nitrogen to mRNA phosphate ratio |
| P188 | Poloxamer 188 |
| RT | room temperature |
| SNIM ® RNA | stabilized Non-immunogenic messenger ribonucleic acid |
| w/o | without |
| PdI | polydispersity index |
| eGFP | enhanced green fluorescent protein |

### 1. Experiment 1—Nanoparticle Quality After Nebulisation for Different Excipients

### 1.1 Materials and Methods

### 1.1.1 Nanoparticle Preparation

Lipidoid nanoparticles were formulated from the cationic lipidoid (dL_05(R), Scheme 1), the helper lipids DPPC (1,2-dipalmitoyl-sn-glycero-3-phosphocholine, Avanti Polar Lipids) and cholesterol (Avanti Polar Lipids) and the PEG lipid DMG-PEG2k (1,2-Dimyristoyl-sn-glycerolmethoxy(polyethylene glycol)-2000, Avanti Polar lipids) at the molar ratios of 8/5.29/4.41/0.88 respectively. Proper volumes of lipid stock solutions in HPLC grade ethanol of concentrations of 50, 20, 20 and 20 mg/mL, respectively, were combined. The formulation process was performed by a rapid solvent exchange. The lipid mixture in ethanol was combined with the mRNA in citrate buffer (10 mM citric acid, 150 mM NaCl, pH 4.5) at a volumetric ratio of 1:4 using a NanoAssemblr benchtop (Precision NanoSystems). The resulting formulation had an mRNA concentration of 0.2 mg/mL with an N/P ratio of 8. After 30 min incubation at RT, the formulation was purified and concentrated by tangential flow filtration (KR₂i TFF System, Repligen) using a 50 kDa filter module (mPES, Repligen) with 50 mM NaCl as dilution and diafiltration buffer. Bioburden reduction and final sterile filtration were performed using 0.8 μm and 0.2 μm syringe filters.

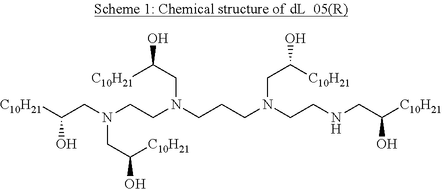

1.1.2 Mixing of Nanoparticle with Excipients

Excipients used in this experiment are listed in Table 3. Dilutions of excipients were prepared at 2% (w/v) excipient in 10% (w/v) sucrose, 50 mM NaCl. Subsequent serial dilutions in sucrose/NaCl buffer yielded in excipient concentrations of 0.2%, 0.02% and 0.002% (w/v). The nanoparticle was mixed with the respective excipient at equal volumes immediately before nebulization.

TABLE 1
List of excipients used in screening experiments.

| Material | Supplier |
|---|---|
| Tween 20 | Sigma (Merck) |
| Tween 80 | Sigma (Merck) |
| Poloxamer 188 (Pluronic F-68) | Sigma-Aldrich |
| Tyloxapol | Sigma (Merck) |
| VitE-PEG1000 (D-α-Tocopherol | Sigma (Merck) |
| polyethylene glycol 1000 succinate) | |

### 1.1.3 Nebulization

Nanoparticle nebulization was carried out in an eFlow nebulizer (Pari). The complete nebulization was performed at RT and time until complete nebulization was measured. The aerosol was collected by allowing condensation in a sample tube at room temperature.

### 1.1.4 Measurement of Complex Size and PdI

Hydrodynamic diameter (Z.average, size) and polydisperity index (PdI) of nanoparticles was measured by dynamic light scattering (DLS) using a Zetasizer Nano-ZS (Malvern Instruments) with an automatic attenuator and reported as intensity particle size distribution. Samples were measured undiluted at 25° C.

### 1.2 Results

This experimental set aimed to compare different classes of additives on their ability to improve particle quality of the nanoparticle after nebulization. As data from the reference (without excipient FIG. 1 “w/o”) demonstrate, the nebulization of nanoparticles results in an undesired increased size (hydrodynamic diameter, Z.average) and polydispersity index (PdI). A significant improvement of the particles overall size and PdI was achieved via addition of certain excipients. FIG. 1 shows the Size and PdI of nanoparticle formulation before and after nebulization of 1 mL at an mRNA concentration of 0.5 mg/mL in 1% excipient, 10% (w/v) sucrose, 50 mM NaCl.

### 2. Experiment 2—Nanoparticle Stability in Presence of Excipients

### 2.1 Materials and Methods

### 2.1.1 Nanoparticle Preparation

See 1.1.1

2.1.2 Mixing of Nanoparticle with Excipients

Excipients used in this experiment are listed in Table 4. Dilutions of excipients were prepared at 20% (My) excipient in 10% (w/v) sucrose, 50 mM NaCl.

TABLE 2
List of excipients.

| Material | Supplier |
|---|---|
| Tween 20 | Sigma (Merck) |
| Tween 80 | Sigma (Merck) |
| Poloxamer 188 (Pluronic F-68) | Sigma-Aldrich |
| Tyloxapol | Sigma (Merck) |
| VitE-PEG1000 (D-α-Tocopherol | Sigma (Merck) |
| polyethylene glycol 1000 succinate) | |

### 2.1.3 Measurement of Encapsulation Efficiency

To determine the encapsulation efficiency, all samples were diluted to 4 μg/mL in water for injection. For “treated samples”, 50 μL of each sample and water as blank control were incubated with 2.67 mg/mL Heparin in 2% (v/v) Triton-X-100 for 15 min at 70° C. in a 96 well plate followed by cooling to RT. For “untreated samples” 50 μL of each sample and water as blank control were diluted with 50 μL water for injection. 100 μL of 100-fold diluted RiboGreen (Quant-iT™ RiboGreen®RNA Assay Kit, ThermoFisher) reagent in 1×TE buffer (10 mM Tris-HCl, 1 mM EDTA, pH 7.5 in diethylpyrocarbonate (DEPC)-treated water) were added into each well and incubated light protected for 5 min at RT. The fluorescence intensity was measured on a Tecan plate reader at excitation/emission wavelength of 785/535 nm, respectively. The encapsulation efficiency was expressed as (100%—(([Emission “untreated sample”]-[Emission “untreated blank”])/([Emission “Treated sample”]-[Emission “treated blank”])*100%).

### 2.2 Results

To allow the usage of the excipient as stabilizer of the nebulization process it should have no negative impact on the nanoparticle itself. To test that an in-use stability study in presence of different excipient concentrations was performed. For that the suspensions were stored for 6 h at RT. The encapsulation efficiency was measured indicating particle integrity. The data is presented in FIG. 2, which shows the encapsulation efficiency of nanoparticle formulation at an mRNA concentration of 2.5 mg/mL in x % (w/v) excipient, 10% (w/v) sucrose, 50 mM NaCl. Data was recorded 6 h after mixing.

### 2.3 Discussion and Conclusion

In presence of Tyloxapol, Tween-20 and Tween-80 the encapsulation efficiency decreases with increasing excipient concentration indicating a loss of nanoparticle integrity. In contrast Poloxamer is well accepted by the formulation. The particle remains intact over a period of 6 h at all tested Poloxamer concentrations.

### 3. Experiment 3—Nanoparticle Nebulization at 2.5 mg/mL in the Presence of Poloxamer

### 3.1 Materials and Methods

### 3.1.1 Nanoparticle Preparation

See section 1.1.10.

### 3.1.2 Addition of Excipient

Poloxamer was added immediately before nebulization (stock concentration: 20% (w/v) P188 in 10% (w/v) sucrose in 50 mM NaCl).

### 3.1.3 Nebulization

See section 1.1.3.

### 3.1.4 Measurement of Complex Size and PdI

See section 1.1.4

### 3.1.5 Measurement of Encapsulation Efficiency

See section 2.1.3

3.1.6 Measurement of mRNA Integrity

The integrity of mRNA in nanoparticles was determined via capillary gel electrophoresis using the Fragment Analyzer (Agilent Technologies). Release of the mRNA from the nanoparticles was carried out at an mRNA concentration of 0.05 mg/mL in 6 μg/μL Heparin (Sigma-Aldrich), 0.2% (v/v) Triton-X-100, 50% (v/v) formamide. Samples were incubated 15 min at 70° C., 300 rpm (Thermomixer, Eppendorf). The mRNA reference was treated accordingly. For sample analysis, treated nanoparticles and mRNA were diluted 1:4 in Diluent Marker (Standard Sensitivity RNA Diluent Marker (15nt), Agilent technologies).

### 3.2 Results

In experiment 1, it has been demonstrated that excipients at 1% (w/v) are able to stabilize nanoparticles during nebulization. Experiment 2 has revealed that Poloxamer in contrast to other tested excipients has no negative influence on nanoparticle stability. In this experiment, the required Poloxamer concentration for the stabilization of a nanoparticle solution with an mRNA concentration of 2.5 mg/mL was evaluated, testing P188 concentrations ranging from 1 to 5% (w/v). For that purpose, 1 mL samples were prepared at the respective concentration, nebulized via an eFlow nebulizer (Pari) and compared to an untreated sample of the same concentration. FIG. 3 shows the biophysical characteristics of 1 mL nanoparticle suspension at 2.5 mg/mL before (untreated) and after nebulization with different Poloxamer concentrations: (a) Size, (b): encapsulation efficiency, (c) relative mRNA integrity

### 3.3 Discussion and Conclusion

Results show that Poloxamer stabilizes the formulation at a nucleic acid concentration of 2.5 mg/mL over a broad concentration range of 1-5% (w/v) excipient. Particle size, encapsulation efficiency as well as mRNA integrity as the three most critical quality attributes remain unaffected by the nebulization process.

### 4. Experiment 4—Nebulization of 25 mg

### 4.1 Materials and Methods

### 4.1.1 Nanoparticle Preparation

See section 1.1.1.

### 4.1.2 Addition of Poloxamer

See section 3.1.2.

### 4.1.3 Nebulization

See section 1.1.3.

### 4.1.4 Measurement of Complex Size and PdI

See section 1.1.4.

### 4.1.5 Measurement of Encapsulation Efficiency

See section 2.1.3.

4.1.6 Measurement of mRNA Integrity

See section 3.1.6

### 4.2 Results

During this experiment the nebulization of a larger volume of nanoparticle solution was tested as this is an additional challenge for the nebulization of a drug. A volume of 10 mL (maximum filling of device) formulated mRNA at a concentration of 2.5 mg/mL was nebulized at a Poloxamer concentration of 5% (w/v). The aerosol was collected and fractionated every 5 min. Fractions were analysed for biophysical properties. The volume was nebulized with an average output rate of approx. 0.3 mL/min. Finally, approx. 500 μL of formulation remained in the reservoir of the nebulizer. FIG. 4 shows the results of the biophysical characterization of fractionated aerosol of 10 mL formulation at an mRNA concentration of 2.5 mg/mL in presence of 5% Poloxamer (Buffer: 5% (w/v) P188, 10% (w/v) sucrose, 50 mM NaCl): (a) Size, (b) PdI, (c) Encapsulation efficiency and (d) rel. mRNA integrity over nebulization time.

### 4.3 Discussion and Conclusion

In the collected fractions the particle size as well as the PdI remained stable (FIGS. 4a and b). The encapsulation efficiency was constantly above 95% (FIG. 4c) and the mRNA integrity remained unaffected (FIG. 4d). Thus, the addition of 5% (w/v) Poloxamer enabled nebulization of 10 mL concentrated formulation (2.5 mg/mL) within 30 min which was not achievable without Poloxamer.

### 5. Experiment 5—Influence of Poloxamer on In Vitro Functionality of the Aerosol Formulation

### 5.1 Materials and Methods

### 5.1.1 Nanoparticle Preparation

See section 1.1.1. using eGFP encoding mRNA

### 5.1.2 Addition of Poloxamer

See section 3.1.2.

### 5.1.3 Nebulization

See section 1.1.3.

### 5.1.4 Measurement of Complex Size and PdI

See section 1.1.4.

### 5.1.5 Measurement of Encapsulation Efficiency

See section 2.1.3.

5.1.6 Measurement of mRNA Integrity

See section 3.1.6.

### 5.1.7 Cell Culture and Transfection

16HBE14o-cells were cultivated in collagen type I (Corning) coated flasks (corning) in MEM+(Thermo Fisher Scientific) GlutaMax™ (Gibco™, Thermo Fisher Scientific) supplemented with heat inactivated fetal bovine serum (FBS, Thermo Fisher Scientific) and penicillin/streptomycin (Pen/Strep, Gibco™, Thermo Fisher Scientific) at 37° C., 5% CO₂. For generation of air liquid interface cultures (ALI) cells were seeded in 250 μL (2.4×10⁵ cells/mL) on the apical side of collagen type I (Corning) coated inserts in 24 well plates. 500 μL medium was added on the basal side and cells were incubated for 72 h to allow attachment. 24 h prior to transfection, the medium in the basal well was exchanged and the medium on the apical side was removed. Transfection was performed following a wash with water for injection adding the respective LNP dose in 25 μL on the apical side of the ALI culture and incubation for 6 h at 37° C., 5% CO₂. After completion of transfection, the cell layer was washed with 200 μL PBS (−/−, Gibco™, Thermo Fisher Scientific) and further maintained as ALI culture.

5.1.8 Quantification of eGFP in ALI Lysates

24 h after transfection, cells were lysed for quantification of intracellular EGFP. ALI inserts were washed with 200 ∥L PBS (−/−, Gibco™, Thermo Fisher Scientific) from the apical side and 500 μL form the basal side. PBS was aspirated and replaced with 100 μL Triton X-100 lysis buffer (0.25 M TRIS-HCl (Carl Roth), 1% Triton-X-100 (Sigma-Aldrich), pH 7.8) complemented with protease inhibitor (cOmplete™, EDTA-free Protease Inhibitor Cocktail, Roche). Inserts were incubated for 20 min at RT on a shaking platform at 600 rpm. Lysates were collected by pipetting up and down several times and stored at −80° C. until analysis. The GFP SimpleStep ELISA® Kit (Abcam ab171581) was used to quantify eGFP levels in cell lysates.

### 5.2 Results

As excipient for stabilization of the nanoparticle the excipient should not have a negative influence on its efficiency on transport of mRNA into cells resulting in expression of the encoded protein. Therefore, the transfection efficiency of the nanoparticle was tested in presence of Poloxamer and Tween-80 before and after nebulization. An mRNA encoding for eGFP protein was used in the formulations allowing a quantification of produced protein. Results are presented in FIG. 5, which shows the eGFP level in celllysates 24 h after transfection of 16HBEo-ALIs with nanoparticles encapsulating eGFP mRNA before (untreated) and after nebulization in presence of 5% (w/v) excipient (Poloxamer or Tween-80). Dotted line: Reference eGFP level after transfection with nanoparticle without excipient.

### 5.3 Discussion and Conclusion

The addition of Tween-80 as excipient for nebulization results in a strong decrease of transfection efficiency. This effect is independent of the nebulization process and thus can be attributed to the presence of the excipient itself. In contrast the addition of Poloxamer has no impact on the transfection efficiency. Protein level before and after nebulization are comparable to level after transfection with the same nanoparticle in absence of excipient.

### 6. Experiment 6—Influence of Poloxamer on In Vivo Efficiency of mRNA Nanoparticles

### 6.1 Materials and Methods

### 6.1.1 Nanoparticle Preparation

See section 1.1.1.

### 6.1.2 Addition of Poloxamer

See section 3.1.2.

### 6.1.3 Nebulization

See section 1.1.3.

### 6.1.4 Measurement of Complex Size and PdI

See section 3.1.4.

### 6.1.5 Measurement of Encapsulation Efficiency

See section 2.1.3.

6.1.6 Measurement of mRNA Integrity

See section 3.1.6.

### 6.1.7 Animal Housing

Mice were housed under specific pathogen free conditions (facility tested negative for any FELASA listed pathogens according to the annual health and hygiene survey 2017) in individually ventilated cages under a circadian light cycle (lights on from 7 a.m. to 7 p.m.). Food and drinking water were provided ad libitum. After arrival, animals were given at least 7 days for acclimatization until they entered the study.

### 6.1.8 Intratracheal Instillation

Animals were anesthetized by the inhalation of pure oxygen containing 4% Isoflurane (Isothesia, Henry Shine). Unconscious animals were intubated using a 20 G catheter shortened to 37 mm. Formulation in a final volume of 50 μL was applied as one drop at the proximal tip of the tubus and thereby aspirated during the physiological inspiratory movement of the animal. Finally, 150 μL of air was applied, to fully empty the catheter.

### 6.1.9 Euthanasia and Necropsy

Animals were set under full anesthesia through intraperitoneal injection of Fentanyl/Midazolam/Medetomidin (0.05/5.0/0.5 mg/kg bw). Subsequently, mice were killed by cervical dislocation. The abdominal cavity was opened in the median axis. For explantation of the lung the small circulation was flushed through the injection of 5 mL PBS through the right ventricle. Subsequently, the heart was dissected from the heart-lung-block. The lung was explanted and snap frozen on dry ice.

6.1.10 Quantification of eGFP in Lung Homogenates

For quantification of eGFP the frozen lung was weighed and the whole organ homogenized. Lysing Matrix D (MP Biomedicals) homogenization tubes were used filled with 500 μL of lysis buffer (0.25 M TRIS (Carl Roth), 0.1% Triton X-100 (Carl Roth), pH 7.8). Homogenization was performed for 3×20 sec in a tissue homogenizer (MP FastPrep-24 Tissue and Cell Homogenizer). Subsequently, the lysates were incubated for 10 min on ice and centrifuged for 10 min at 4° C. (Mikro 22R centrifuge, Hettich Zentrifugen) at 20.000×g. The GFP SimpleStep ELISA® Kit (Abcam ab171581) was used to quantify eGFP. Levels of eGFP were correlated with lung weights and reported in ng protein/g tissue.

### 6.2 Results

In order to serve as excipient for the stabilization of nanoparticles used as drug the excipient should not negatively influence the transfection efficiency of those nanoparticles in vivo. During this study the transfection efficiency of a nanoparticle in presence of excipient was measured after i.t. application in mice. The efficiency was determined via quantification of the mRNA encoded eGFP 24 h after treatment. FIG. 6 shows the results of the treatment with three different doses in presence and absence of Poloxamer, in particular the eGFP level upon i.t. instillation of nanoparticle in 10% (w/v) sucrose, 50 mM NaCl without (w/o) and with excipient.

### 6.3 Discussion and Conclusion

eGFP level measured in the mouse lung were equal in presence and absence of Poloxamer at the respective dose level. The analysis of the generated data leads to the conclusion that Poloxamer does not have a negative influence on the transfection efficiency of the nanoparticle in vivo.

### 7. Experiment 7—Addition of Poloxamer During Nanoparticle Downstream Processing Improves Nanoparticle Quality

### 7.1 Materials and Methods

### 7.1.1 Nanoparticle Preparation

See section 1.1.1

### 7.1.2 Measurement of Complex Size and PdI

See section 1.1.4.

### 7.2 Results

After complex formation via solvent exchange the buffer components required for this step (EtOH and citric acid) have to be removed. The standard procedure for this step as well as for concentration adjustment of the formulation is tangential flow filtration. During this treatment the nanoparticle undergoes stress conditions resulting in loss of particle quality (e.g. aggregation). During this experiment Poloxamer was used to stabilize the nanoparticle during this process step. For that purpose nanoparticles were formulated under standard conditions. In one set Poloxamer was added at a concentration of 0.5% (w/v) prior to TFF processing. The quality of the particle was determined via measurement of particle size after mixing and after processing. FIG. 7 shows the Size and PdI of nanoparticle formulation before and after nebulization processing in presence or absence of Poloxamer

### 7.3 Discussion and Conclusion

As shown in FIG. 7 the particle size as well as the polydispersity increases during TFF processing in absence of Poloxamer indicating particle aggregation. In presence of Poloxamer particle size as well as polydispersity remain stable during processing. Thus, the addition of Poloxamer clearly improves particle quality during processing of nanoparticles.

### 8. Experiment 8—Nanoparticle Quality of ICE Based Nanoparticles After Nebulization in Absence and Presence of Poloxamer

### 8.1 Materials and Methods

### 8.1.1 Nanoparticle Preparation

The lipid nanoparticle was formulated from the cationic lipid (ICE (Imidazole Cholesterol Ester)), the helper lipid DOPE (1,2-Dioleoyl-sn-glycero-3-phosphoethanolamine, Avanti Polar Lipids) and the PEG lipid DMG-PEG2k (1,2-Dimyristoyl-sn-glycerolmethoxy(polyethylene glycol)-2000, Avanti Polar lipids) at the molar ratios of 60/35/5 respectively. Proper volumes of lipid stock solutions in HPLC grade ethanol at 10 mg/mL were combined. The formulation process was performed by a rapid solvent exchange. The lipid mixture in ethanol was combined with the mRNA in citrate buffer (10 mM citric acid, 150 mM NaCl, pH 4.5) at a volumetric ratio of 1:4 using a NanoAssemblr benchtop (Precision NanoSystems). The resulting formulation had an mRNA concentration of 0.2 mg/mL with an N/P ratio of 4. After 30 min incubation at RT, the formulation was purified and concentrated by tangential flow filtration (KR₂i TFF System, Repligen) using a 100 kDa filter module (mPES, Repligen) with 25 mM NaCl as dilution and diafiltration buffer. Bioburden reduction and final sterile filtration were performed using 0.8 μm and 0.2 μm syringe filters.

### 8.1.2 Addition of Excipient

Poloxamer was added immediately before nebulization (stock concentration: 20% (w/v) Poloxamer 188 in 25 mM NaCl) resulting in a concentration of 0.5 mg/mL mRNA in 5% (w/v) Poloxamer 188, 25 mM NaCl.

### 8.1.3 Nebulization

See section 1.1.3

### 8.1.4 Measurement of Complex Size and PdI

See section 1.1.4

### 8.2 Results

Experiment 1 and 3 have shown that Poloxamer stabilizes nanoparticles that are based on the cationic lipidoid dL_05(R) during nebulization. This experimental set aimed to test, whether the addition of Poloxamer can also preserve particle quality of other commonly used nanoparticles, based on a different cationic lipid (ICE) during nebulization. The data from the reference (without excipient FIG. 8 “w/o”) demonstrate, that nebulization of the nanoparticles leads to an increase in size (hydrodynamic diameter, Z.average) and polydispersity index (PdI). The addition of 5% (w/v) Poloxamer prevents this effect and preserves particle quality. FIG. 8 shows the size and PdI of 1 mL nanoparticle suspension at an mRNA concentration of 0.5 mg/mL before and after nebulization with or without Poloxamer.

### 8.3 Discussion and Conclusion

The presence of Poloxamer also stabilizes lipid nanoparticles based on the cationic lipid ICE during nebulization and prevents an increase in size (hydrodynamic diameter, Z.average) and polydispersity index (PdI).

### 9. Experiment 9—Nanoparticle Quality of DLin-MC3-DMA Based Nanoparticles After Nebulization in Absence and Presence of Poloxamer

### 9.1 Materials and Methods

### 9.1.1 Nanoparticle Preparation

The lipid nanoparticle was formulated from the cationic lipid (DLin-MC3-DMA), the helper lipids DSPC (1,2-Distearoyl-sn-glycero-3-phosphocholine, Avanti Polar Lipids) and cholesterol (Avanti Polar Lipids) and the PEG lipid DMPE-PEG2k (1,2-Dimyristoyl-sn-glycero-3-phosphoethanolamine-polyethylene glycol 2000, Avanti Polar lipids) at the molar ratios of 50/10/38.5/1 respectively. Proper volumes of lipid stock solutions in HPLC grade ethanol at 10 mg/mL were combined. The formulation process was performed by a rapid solvent exchange. The lipid mixture in ethanol was combined with the mRNA in citrate buffer (50 mM citric acid, 160 mM NaCl, pH 3) at a volumetric ratio of 1:3 using a NanoAssemblr benchtop (Precision NanoSystems). The resulting formulation had an mRNA concentration of 0.2 mg/mL with an N/P ratio of 3. After 30 min incubation at RT, the formulation was purified and concentrated by tangential flow filtration (KR₂i TFF System, Repligen) using a 100 kDa filter module (mPES, Repligen) with PBS as dilution and diafiltration buffer. Bioburden reduction and final sterile filtration were performed using 0.8 μm and 0.2 μm syringe filters.

### 9.1.2 Addition of Excipient

Poloxamer was added immediately before nebulization (stock concentration: 20% (w/v) Poloxamer 188 in PBS) resulting in a concentration of 0.5 mg/mL in 5% (w/v) Poloxamer 188 in PBS.

### 9.1.3 Nebulization

See section 1.1.3

### 9.1.4 Measurement of Complex Size and PdI

See section 1.1.4

### 9.2 Results

Experiment 1 and 3 have shown, that Poloxamer stabilizes nanoparticles, that are based on the cationic lipidoid dL_05(R) during nebulization. This experimental set aimed to test, whether the addition of Poloxamer can also preserve particle quality of other commonly used nanoparticles during nebulization. A well described nanoparticle based on the cationic lipid DLin-MC3-DMA was chosen as model. In absence of excipient (without Poloxamer, FIG. 9 “w/o”) nebulization of the nanoparticles leads to an increase in size (hydrodynamic diameter, Z.average) and polydispersity index (PdI). The addition of 5% (w/v) Poloxamer prevents this effect and preserves particle quality. FIG. 9 shows the size and PdI of 1 mL nanoparticle suspension at 0.5 mg/mL before and after nebulization with or without Poloxamer.

### 9.3 Discussion and Conclusion

The presence of Poloxamer stabilizes lipid nanoparticles based on the cationic lipid DLin-MC3-DMA during nebulization. Addition of Poloxamer prevents an increase in size (hydrodynamic diameter, Z.average) and polydispersity index (PdI).

### Description of the Figures

FIG. 1 shows the size and PdI of nanoparticle formulation before and after nebulization of 1 mL at an mRNA concentration of 0.5 mg/mL in 1% excipient, 10% (My) sucrose, 50 mM NaCl.

FIG. 2 shows the encapsulation efficiency of nanoparticle formulation at an mRNA concentration of 2.5 mg/mL in x % (w/v) excipient, 10% (w/v) sucrose, 50 mM NaCl. Data was recorded 6 h after mixing.

FIG. 3 shows the biophysical characteristics of 1 mL nanoparticle suspension at 2.5 mg/mL before (untreated) and after nebulization with different Poloxamer concentrations: (a) Size, (b): encapsulation efficiency, (c) relative mRNA integrity.

FIG. 4 shows the results of the biophysical characterization of fractionated aerosol of 10 mL formulation at an mRNA concentration of 2.5 mg/mL in presence of 5% Poloxamer (Buffer: 5% (w/v) P188, 10% (w/v) sucrose, 50 mM NaCl): (a) Size, (b) PdI, (c) Encapsulation efficiency and (d) rel. mRNA integrity over nebulization time.

FIG. 5 shows the eGFP level in celllysates 24 h after transfection of 16HBEo-ALIs with nanoparticles encapsulating eGFP mRNA before (untreated) and after nebulization in presence of 5% (w/v) excipient (Poloxamer or Tween-80). Dotted line: Reference eGFP level after transfection with nanoparticle without excipient.

FIG. 6 shows the eGFP level upon i.t. instillation of nanoparticle in 10° A) (w/v) sucrose, 50 mM NaCl without (w/o) and with excipient.

FIG. 7 shows the size and PdI of nanoparticle formulation before and after nebulization processing in presence or absence of Poloxamer.

FIG. 8 shows the size and of nanoparticle formulation (with ICE as cationic lipid) before and after nebulization of 1 mL at an mRNA concentration of 0.5 mg/mL in the presence or absence of 5% (w/v) Poloxamer.

FIG. 9 shows the size and PdI of nanoparticle formulation (with DLin-MC3-DMA as cationic lipid) before and after nebulization of 1 mL at an mRNA concentration of 0.5 mg/mL in the presence or absence of 5% (w/v) Poloxamer.

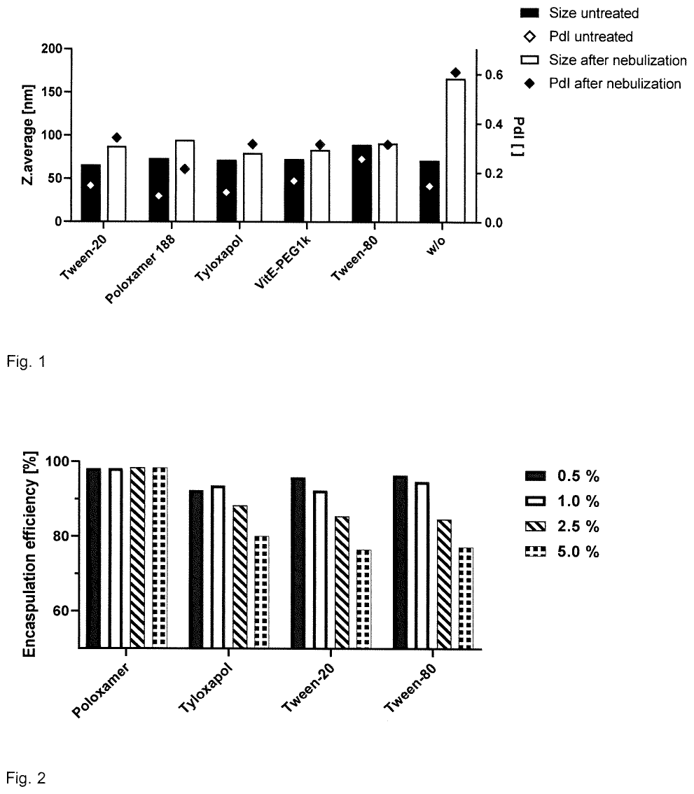

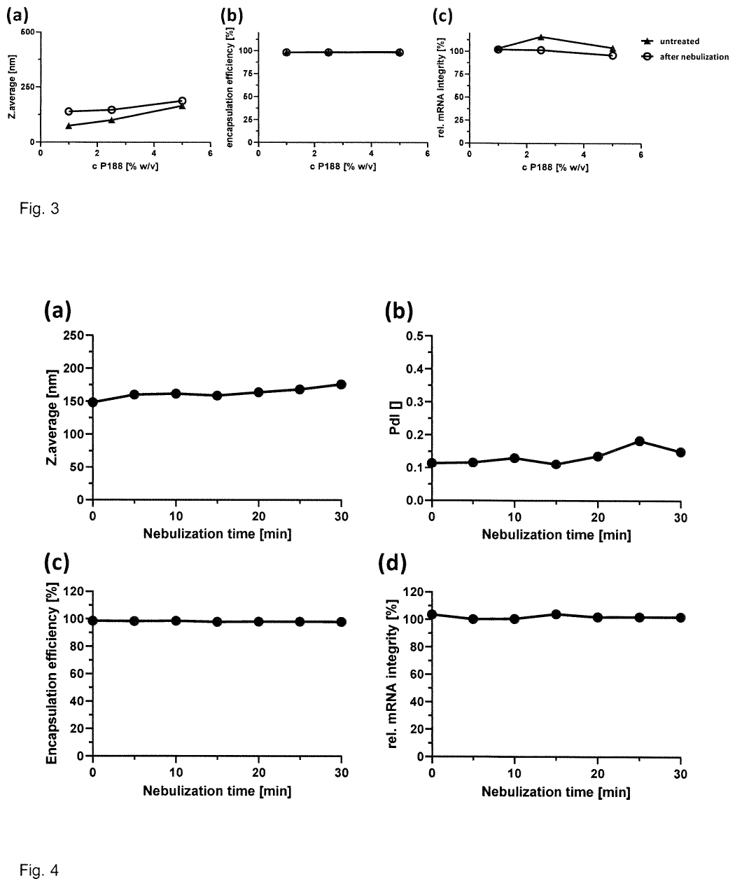

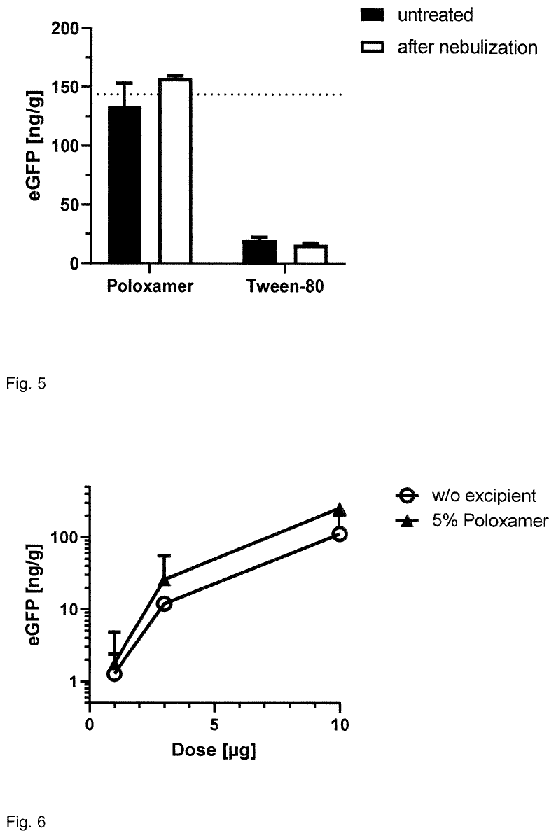

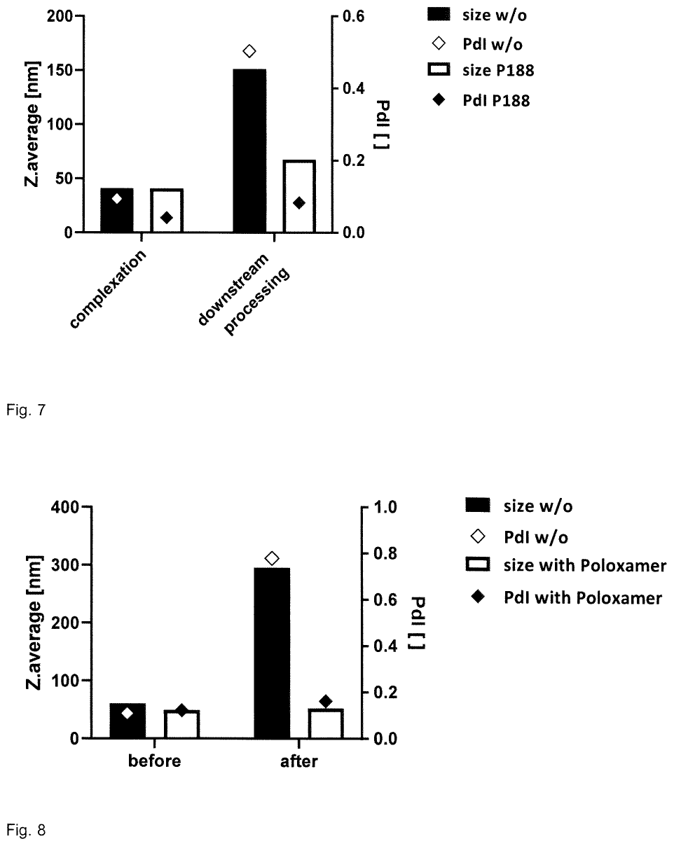

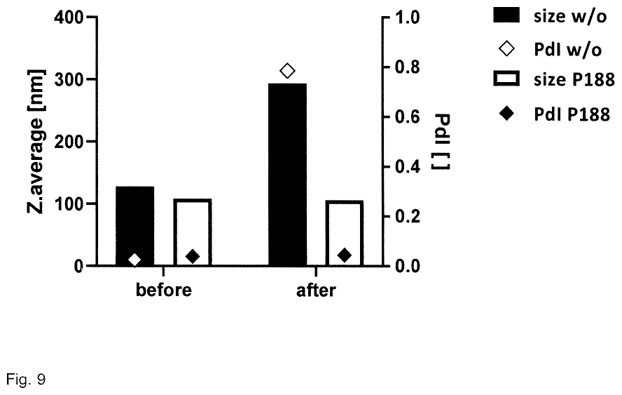

1. An aqueous suspension formulation for aerosol formation, said suspension formulation comprising lipid or lipidoid nanoparticles which are suspended in an aqueous vehicle solution,
wherein the lipid or lipidoid nanoparticles comprise the following components (a) and (b):
(a) a nucleic acid and
(b) an ionizable lipid or an ionizable lipidoid;
and wherein the aqueous vehicle solution comprises a triblock copolymer which contains one poly(propylene oxide) block and two poly(ethylene oxide) blocks.

2. The suspension formulation in accordance with claim 1, wherein the concentration of the nucleic acid in the suspension formulation ranges from 0.01 to 10 mg/mL, based on the total volume of the suspension formulation.

3. The suspension formulation in accordance with claim 1, wherein the nanoparticles have a Z-average diameter, as determined by dynamic light scattering, in the range of 10 to 500 nm.

4. The suspension formulation in accordance with claim 1, wherein the nanoparticles further comprise one or more of the following components (c1) to (c6):
(c1) a non-ionizable lipid having a sterol structure;
(c2) a phosphoglyceride lipid;
(c3) a PEG-conjugated lipid;
(c4) a polysarcosine-conjugated lipid
(c5) a PASylated lipid; and
(c6) a cationic polymer.

5. The suspension formulation in accordance with claim 1, wherein the nanoparticles comprise:
30 to 65 mol % of the ionizable lipid or ionizable lipidoid (b), and one or more of the following components:
10 to 50 mol % of the lipid having a sterol structure (c1),
4 to 50 mol % of the phosphoglyceride lipid (c2),
0.5 to 10 mol % of one of the PEG-conjugated lipid (c3), the polysarcosine-conjugated lipid (c4) and the PASylated lipid (c5), or of any combination thereof,
0.5 to 10 mol % of the cationic polymer (c6),
such that the sum of (b) and (c1) to (c6) amounts to 100 mol %.

6. The suspension formulation in accordance with claim 1, wherein the nanoparticles comprise an ionizable lipidoid (b) of the following formula (b-1), 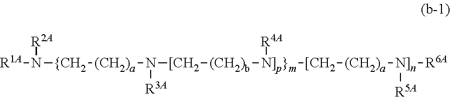
wherein:
a is 1 and b is an integer of 2 to 4; or a is an integer of 2 to 4 and b is 1,
p is 1 or 2,
m is 1 or 2; n is 0 or 1 and m+n is≥2; and
R_{1A} to R_{6A} are independently of each other selected from:
hydrogen; —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A}; —CH₂—R_{7A}; —C(NH)—NH₂; a poly(ethylene glycol) chain; and a receptor ligand; wherein R_{7A} is selected from C3-C18 alkyl and C3-C18 alkenyl having one C—C double bond;
provided that at least two residues among R_{1A} to R_{6A} are selected from —CH₂—CH(OH)—R_{7A}, —CH(R_{7A})—CH₂—OH, —CH₂—CH₂—(C═O)—O—R_{7A}, —CH₂—CH₂—(C═O)—NH—R_{7A} and —CH₂—R_{7A}, wherein R_{7A} is selected from C3-C18 alkyl or C3-C18 alkenyl having one C—C double bond;
or a protonated form thereof, wherein one or more of the nitrogen atoms contained in the compound of formula (b-1) are protonated to provide a compound carrying a positive charge.

7. The suspension formulation in accordance with claim 1, wherein the triblock copolymer is an A-B-A triblock copolymer which contains one poly(propylene oxide) block B of formula (p-1): 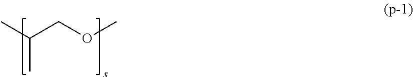
wherein s is an integer of 15 to 67, preferably 20 to 40 and
two poly(ethylene oxides) blocks A of formula (p-2): 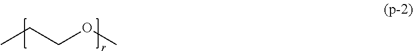
wherein r is, independently for each block, an integer of 2 to 130.

8. The suspension formulation in accordance with claim 1, which comprises the triblock copolymer at a concentration of 0.05 to 5% (w/v, at a temperature of 25° C.), based on the total volume of the suspension formulation.

9. A nebulizer, which comprises a compartment wherein the aqueous suspension formulation for aerosol formation in accordance with claim 1 is contained.

10. An aerosol comprising aerosol droplets dispersed in a gas phase, wherein the aerosol droplets comprise lipid or lipidoid nanoparticles and an aqueous vehicle solution for the nanoparticles,
wherein the lipid or lipidoid nanoparticles comprise the following components (a) and (b):
(a) a nucleic acid and
(b) an ionizable lipid or an ionizable lipidoid;
and wherein the aqueous vehicle solution comprises a triblock copolymer which contains one poly(propylene oxide) block and two poly(ethylene oxide) blocks.

11. The aerosol in accordance with claim 10, wherein the mass median aerodynamic diameter (MMAD) of the aerosol droplets ranges from 2 to 10 μm.

12. The aerosol in accordance with claim 11, wherein the nanoparticles have a Z-average diameter, as determined by dynamic light scattering, in the range of 10 to 500 nm.

13. The aerosol in accordance with claim 10, which is obtainable by nebulization of an aqueous suspension formulation in accordance with claim 1.

14. The aqueous suspension formulation in accordance with claim 1 for use as a medicament, wherein the aqueous suspension formulation is to be nebulized and the aerosol provided by nebulization is to be administered to a subject.

15. A method for treating or preventing a disease or disorder via a nucleic acid-based therapy, wherein the treatment or prevention comprises the nebulization of the aqueous suspension formulation in accordance with claim 1 and the administration of the aerosol provided by nebulization to or via the respiratory tract of a subject.

16. The method in accordance with claim 15, wherein the disease or disorder to be treated or prevented is a pulmonary disease.

17. The method in accordance with claim 15, wherein the administration of the aerosol occurs via pulmonary administration or nasal administration.

18. The suspension formulation in accordance with claim 7, wherein r is, independently for each block, an integer of 50 to 100.

19. The suspension formulation in accordance with claim 7, wherein r is, independently for each block, an integer of 60 to 90.

20. The aerosol in accordance with claim 11, wherein the nanoparticles have a Z-average diameter, as determined by dynamic light scattering, in the range of 10 to 250 nm.
